(19) United States
(12) Patent Application Publication
(10) Pub. No.: US 2023/0313229 A1
(43) Pub. Date: Oct. 5, 2023

(54) Genetic Construct

(71) Applicant: Instituto de Medicina Molecular João Lobo Antunes, Lisboa (PT)

(72) Inventors: Ana Rita Freitas Martins de Matos FRAGOSO, Lisboa (PT); João Pedro Taborda BARATA, Lisboa (PT)

(73) Assignee: Instituto de Medicina Molecular João Lobo Antunes, Lisboa (PT)

(21) Appl. No.: 18/043,276

(22) PCT Filed: Aug. 25, 2021
(86) PCT No.: PCT/PT2021/050028

§ 371 Date: Feb. 27, 2023

(30) Foreign Application Priority Data
Aug. 27, 2020 (GB) 2013466.4

(51) Int. Cl. C12N 15/86

(52) U.S. Cl. CPC C12N 15/86; C12N 2310/141; C12N 2320/30; C12N 2830/001

(57) ABSTRACT

The invention relates to genetic constructs comprising at least one microRNA target site, and vectors comprising such constructs. The genetic constructs and vectors can be used in diagnosis and therapy of a range of disorders, including cancers, for example T-cell acute lymphoblastic leukaemia (T-ALL).

The present invention relates to genetic constructs and vectors. The invention extends to pharmaceutical compositions comprising the genetic constructs and vectors, and to methods of using the genetic constructs and vectors in diagnosis and therapy. The invention is particularly, although not exclusively, concerned with diagnosing and treating T-cell acute lymphoblastic leukaemia (T-ALL).

Over the last few decades, there has been a significant increase in the knowledge and understanding of the molecular biology of various disorders. This has led to the increasing use of genetic-based approaches in both the treatment and diagnosis of conditions, such as cancer, which enables more precise treatment of conditions and also greater diagnostic specificity, enabling the identification of disease subsets that would otherwise go undetected.

Intense efforts in cancer research have resulted in a better understanding of the mechanisms that drive cancer development. Consequently, there has been a shift on how cancer therapy is perceived, leading to the search for and development of target-specific agents (1). However, despite this shift, cancer patients are still predominantly treated with non-specific cytotoxic drugs. Even though most cancer drivers are known, targeting these genes has been very challenging. Given the important functions that most of these genes have in normal tissues, it is difficult to target them without causing significant toxicity to normal cells. Therefore, it is imperative to develop alternative therapeutic strategies.

T-ALL is an aggressive haematological malignancy frequently associated with poor prognosis factors, such as the infiltration of the central nervous system. T-ALL accounts for 15% and 25% of paediatric and adult acute lymphoblastic leukaemia, respectively (2) and, although the outcome of T-ALL patients has improved over recent years, the prognosis of patients with resistant or relapsed disease remains very poor (3,4). Currently, the low response rates and the high toxicity of the existing therapies underline the need to develop more specific and effective therapeutic strategies (3).

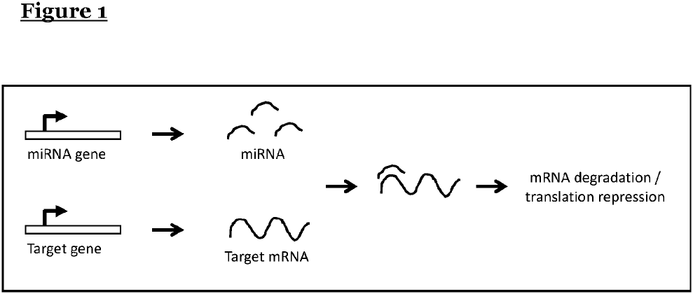

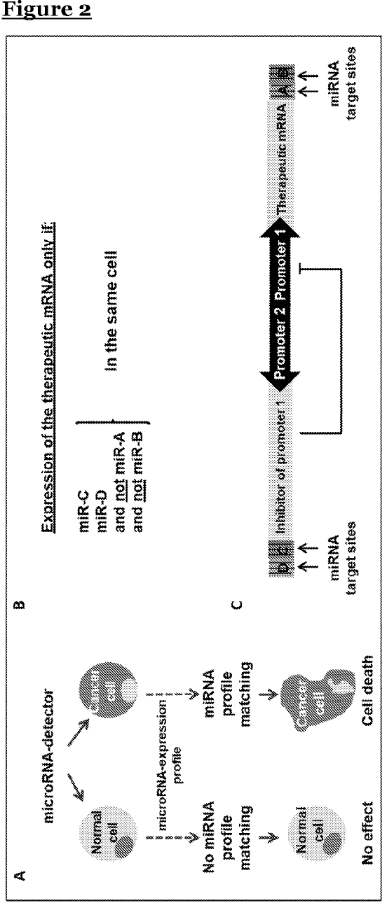

MicroRNAs (miRs) are ~22 nucleotides small non-coding RNAs that regulate gene expression post-transcriptionally (5) by binding to complementary sequences usually located in 3′ UTR of their target mRNAs (6) (see FIG. 1). MicroRNAs regulate several biological processes as well as human diseases including cancer (5). Moreover, functional studies have shown that microRNAs partake in tumorigenesis acting either as tumour suppressors or as oncogenes (7).

Importantly, profiling studies have shown that some microRNAs are expressed in a highly tissue/cell specific fashion (8, 9). Such tissue/cell-type specific microRNA expression has been exploited to negatively regulate transgene expression. Addition of artificial sequences recognized by a specific microRNA to a transgene will induce its post-transcriptional silencing. This concept was first developed by Brown and colleagues (10). By constructing a transgene expression lentiviral vector that incorporates target sequences for a hematopoietic-cell specific microRNA, the authors built a system that allows transgene expression in non-hematopoietic cells while suppressing its expression in hematopoietic lineages. More recently, this strategy has been explored in different disease settings for therapeutic gene delivery (11-13).

In cancer, microRNA expression profiles have also been shown to distinguish tumour from normal cells and to discriminate tumours of different developmental origin and having a differentiation state (8,9, 14, 15). The inventors, therefore, set out to take advantage of T-ALL-specific microRNA expression profiles, and have developed a recombinant genetic construct, which behaves as a so-called microRNA “detector system”. The genetic construct is capable of firstly using the microRNA profile to identify cancerous or tumourous T-ALL cells in a patient and distinguish the leukaemia cells from the patient’s normal, healthy cells, and secondly regulate the expression of a therapeutic gene (e.g. an apoptosis-inducing gene encoding a protein which causes tumour cell death) or a marker gene (e.g. a reporter gene encoding a protein marker) in only the T-ALL cells, and not the healthy cells.

Thus, in a first aspect of the invention, there is provided a genetic construct comprising:
(i) a first promoter operably linked to a first nucleic acid sequence encoding a therapeutically active molecule or a reporter molecule, wherein the first nucleic acid sequence comprises at least one microRNA (miRNA) target site; and
(ii) a second promoter operably linked to a second nucleic acid sequence encoding an inhibitor of the first promoter and/or the therapeutically active molecule or reporter molecule, and wherein the second nucleic acid sequence comprises at least one miRNA target site,
wherein the at least one miRNA target site of the first and second nucleic acid sequences are different.

Advantageously, the construct of the invention harnesses the predetermined microRNA profile specific to target and, in a tumour context, kill unhealthy cells in a patient. When expressed inside a patient’s cell, the construct determines whether the cell is an unhealthy cell, in which case, it induces cell death, or whether the cell is a healthy cell, in which case no cell death is induced. Thus, a positive match between the expression of specific microRNAs inside an unhealthy cell and the construct results in the delivery of a therapeutic gene to target cells, inducing their death (as shown in FIG. 2A). The intrinsic ability of the construct to distinguish between an unhealthy cell and a healthy cell within the body of the patient is a hallmark of the technology, a valuable tool in precision oncology, and a significant advantage over the prior art, because it provides the best possible efficacy and safety outcomes for patients. Indeed, the construct of the invention has the potential to effectively circumvent the lack of specificity that is associated with standard gene therapy techniques.

Furthermore, with the construct of the invention, a therapeutically active molecule can be delivered with high specificity and efficiency, using just one single vector. Additionally, only one construct (i.e. the construct of the invention) is delivered to the target cell, thereby ensuring co-localisation of (i) the therapeutically active molecule or a reporter molecule, and (ii) the inhibitor of the first promoter and/or the therapeutically active molecule or reporter molecule. This means that significantly lower doses of construct require administration compared to when using multiple constructs and ensuring co-delivery into the same cells. Additionally, this single construct does not require induction with external factors or cues to drive the expression of the therapeutic molecule. As such, the construct of the invention is significantly advantageous over the prior art, as it results in a much simpler and more effective system.

Preferably, the at least one miRNA target site of the second nucleic acid sequence is a target site of an miRNA that is different to an miRNA capable of targeting the at least one miRNA target site of the first nucleic acid sequence.

It will be appreciated that the construct of the invention, therefore, comprises a first expression cassette comprising the first promoter and the first nucleic acid sequence, and a second expression cassette comprising the second promoter and the second nucleic acid sequence.

It will be appreciated that the first nucleic acid sequence may comprise more than one species or type of miRNA target sequence.

Preferably, the first nucleic acid sequence comprises at least one miRNA target site (or at least one species of miRNA target site). Preferably, the first nucleic acid sequence comprises at least two miRNA target sites (or at least two species of miRNA target site). Preferably, the first nucleic acid sequence comprises at least three miRNA target sites (or at least three species of miRNA target site). Preferably, the first nucleic acid sequence comprises at least four miRNA target sites (or at least four species of miRNA target site). Preferably, the first nucleic acid sequence comprises at least five miRNA target sites (or at least five species of miRNA target site). Preferably, the miRNA target sites present in the first nucleic acid sequence are target sites for different miRNAs species. The more miRNA target sites that are present in the first nucleic acid sequence, then the more tightly regulated gene expression will be. However, it will be appreciated that, for the construct to function adequately, a minimum of only one miRNA target site in each of the first and second nucleic acid sequences is required, provided that they are target sites for different miRNAs.

It will be appreciated that there may be more than one copy of each miRNA target site, i.e. each miRNA target site species may comprise at least one duplication of the target site. Accordingly, preferably there is at least one copy of each miRNA target site, i.e. each species. Preferably, there is at least two copies of each miRNA target site, i.e. each species. Preferably, there is at least three copies of each miRNA target site, i.e. each species. Preferably, there is at least four copies of each miRNA target site, i.e. each species. Preferably, there is at least five copies of each miRNA target site, i.e. each species.

Preferably, the at least one miRNA target site present in the first nucleic acid sequence is a target site for an miRNA, the expression of which is absent or decreased or down-regulated in a diseased cell when compared to a healthy cell.

The identification of miRNAs specifically absent or decreased in a cancer cell when compared with a healthy cell may be obtained by the analysis of differential expressed miRNA between the cancer and healthy cells assuming the following criteria:
1) the 5^{th}, 10^{th}, 15^{th}, 20^{th},25^{th}, 30^{th} or 35^{th} percentile of the higher-expressing group is higher than the 60^{th}, 65^{th}, 70^{th}, 75^{th}, 80^{th}, 85^{th}, 90^{th} or 95^{th} percentile of the lower-expressing group, preferably, the 25th, percentile of the higher-expressing group is higher than the 75th, percentile of the lower-expressing group; and
2) miRNA has expression values for at least 60%, 65%, 75%, 80%, 85%, 90% or 95% of all the healthy samples analysed. Preferably, miRNA has expression values for at least at least 75% of all the healthy samples analysed.

Preferably, the at least one miRNA target site present in the first nucleic acid sequence is a target site for a miRNA that is not expressed in a diseased cell, or to very low or undetectable levels. Preferably, the at least one miRNA target site present in the first nucleic acid is a target site for a miRNA, which is specifically expressed in healthy cells. Preferably, the at least one miRNA target site present in the first nucleic acid is a target site for a miRNA, which is substantially expressed in healthy cells only. Preferably, the at least one miRNA target site present in the first nucleic acid is a target site for a miRNA, which is ubiquitously expressed in healthy cells.

Down-regulated or decreased miRNA expression in a diseased cell may be considered to be at least 5%, 10%, 15% or 20% less than occurs in a healthy cell. Preferably, down-regulated or decreased miRNA expression in a diseased cell may be considered to be at least 25%, 30%, 35% or 40% less than occurs in a healthy cell. Preferably, down-regulated or decreased miRNA expression in a diseased cell may be considered to be at least 50%, 55%, 60% or 65% less than occurs in a healthy cell. Preferably, down-regulated or decreased miRNA expression in a diseased cell may be considered to be at least 70%, 75%, 80% or 85% less than occurs in a healthy cell. Preferably, down-regulated or decreased miRNA expression in a diseased cell may be considered to be at least 90%, 95%, 96%, 97%, 98%, 99% or 100 % less than occurs in a healthy cell.

Preferably, the diseased cell is a cancer cell. More preferably, the diseased cell is a T-cell acute lymphoblastic leukaemia (T-ALL) cell. By way of example only, miRNA₁₅₃, miRNA128a, miR-3687, miR-92a-2-5p, miR-20b-3p, miR-6087, miR-106a-3p, miR-7704, miR-5701, miR-766-5P, miR-3609, miR-3615, and/or miR-4746-5p are up-regulated in T-ALL cells (i.e. higher expression levels than in healthy cells), whereas miRNA29a, miRNA149, miR-539-5p, miR-487a-3p, miR-655-3p, miR-411-3p miR-377-5p, miR-337-5p, miR-31-3p, miR-214-5p, miR-1185-5p, miR-483-5p, miR-365a-3p, miR-127-3p, miR-574-3P, and/or miR-125b-5p are down-regulated in T-ALL cells (i.e. lower expression levels than in healthy cells).

Preferably, therefore, the at least one miRNA target site present in the first nucleic acid sequence comprises a miRNA29a, miRNA149, miR-539-5p, miR-487a-3p, miR-655-3P, miR-411-3p miR-377-5P, miR-337-5p, miR-31-3P, miR-214-5p, miR-1185-5p, miR-483-5P, miR-365a-3p, miR-127-3p, miR-574-3p, and/or miR-125b-5p target site. Preferably, the at least one miRNA target site present in the first nucleic acid sequence comprises at least one, two, three, four or five copies of each of the miRNA29a, miRNA149, miR-539-5p, miR-487a-3P, miR-655-3p, miR-411-3p miR-377-5P, miR-337-5p, miR-31-3p, miR-214-5p, miR-1185-5p, miR-483-5p, miR-365a-3p, miR-127-3p, miR-574-3p and miR-125b-5p target sites. More preferably, the at least one miRNA target site present in the first nucleic acid sequence comprises four copies of each of the miRNA29a, miRNA149, miR-539-5P, miR-487a-3p, miR-655-3p, miR-411-3p miR-377-5p, miR-337-5p, miR-31-3p, miR-214-5p, miR-1185-5P, miR-483-5p, miR-365a-3p, miR-127-3p, miR-574-3p and miR-125b-5p target sites.

Preferably, the at least one miRNA target site present in the first nucleic acid sequence comprises a miRNA29a target site, which is provided herein as SEQ ID No: 1, as follows:

TAACCGATTTCAGATGGTGCTA
[
SEQ ID No: 1]

Thus, preferably the at least one miRNA target site present in the first nucleic acid sequence comprises a miRNA29a target site which comprises or consists of a sequence as substantially set out in SEQ ID No: 1, or a variant or fragment thereof.

Preferably, the at least one miRNA target site present in the first nucleic acid sequence comprises a miR149 target site, which is provided herein as SEQ ID No: 2, as follows:

GGGAGTGAAGACACGGAGCCAGA
[
SEQ ID No: 2]

Thus, preferably the at least one miRNA target site present in the first nucleic acid sequence comprises a miR149 target site which comprises or consists of a sequence as substantially set out in SEQ ID No: 2, or a variant or fragment thereof.

Preferably, the at least one miRNA target site present in the first nucleic acid sequence comprises a miR-539-5p target site, which is provided herein as SEQ ID No: 28, as follows:

ACACACCAAGGATAATTTCTCC
[S
EQ ID No: 28]

Thus, preferably the at least one miRNA target site present in the first nucleic acid sequence comprises a miR-539-5p target site which comprises or consists of a sequence as substantially set out in SEQ ID No: 28, or a variant or fragment thereof.

Preferably, the at least one miRNA target site present in the first nucleic acid sequence comprises a miR-487a-3p target site, which is provided herein as SEQ ID No: 29, as follows:

AACTGGATGTCCCTGTATGATT
[SEQ ID
No: 29]

Thus, preferably the at least one miRNA target site present in the first nucleic acid sequence comprises a miR-487a-3p target site which comprises or consists of a sequence as substantially set out in SEQ ID No: 29, or a variant or fragment thereof.

Preferably, the at least one miRNA target site present in the first nucleic acid sequence comprises a miR-655-3p target site, which is provided herein as SEQ ID No: 30, as follows:

AAAGAGGTTAACCATGTATTAT
[SEQ ID
No: 30]

Thus, preferably the at least one miRNA target site present in the first nucleic acid sequence comprises a miR-655-3p target site which comprises or consists of a sequence as substantially set out in SEQ ID No: 30, or a variant or fragment thereof.

Preferably, the at least one miRNA target site present in the first nucleic acid sequence comprises a miR-411-3p target site, which is provided herein as SEQ ID No: 31, as follows:

GGTTAGTGGACCGTGTTACATA
[SEQ ID
No: 31]

Thus, preferably the at least one miRNA target site present in the first nucleic acid sequence comprises a miR-411-3p target site which comprises or consists of a sequence as substantially set out in SEQ ID No: 31, or a variant or fragment thereof.

Preferably, the at least one miRNA target site present in the first nucleic acid sequence comprises a miR-377-5p target site, which is provided herein as SEQ ID No: 32, as follows:

GAATTCACCAAGGGCAACCTCT
[SEQ ID
No: 32]

Thus, preferably the at least one miRNA target site present in the first nucleic acid sequence comprises a miR-377-5p target site which comprises or consists of a sequence as substantially set out in SEQ ID No: 32, or a variant or fragment thereof.

Preferably, the at least one miRNA target site present in the first nucleic acid sequence comprises a miR-337-5p target site, which is provided herein as SEQ ID No: 33, as follows:

AACTCCTGTATGAAGCCGTTC
[SEQ ID N
o: 33]

Thus, preferably the at least one miRNA target site present in the first nucleic acid sequence comprises a miR-337-5p target site which comprises or consists of a sequence as substantially set out in SEQ ID No: 33, or a variant or fragment thereof.

Preferably, the at least one miRNA target site present in the first nucleic acid sequence comprises a miR-31-3p target site, which is provided herein as SEQ ID No: 34, as follows:

ATGGCAATATGTTGGCATAGCA
[SEQ ID
No: 34]

Thus, preferably the at least one miRNA target site present in the first nucleic acid sequence comprises a miR-31-3p target site which comprises or consists of a sequence as substantially set out in SEQ ID No: 34, or a variant or fragment thereof.

Preferably, the at least one miRNA target site present in the first nucleic acid sequence comprises a miR-214-5p target site, which is provided herein as SEQ ID No: 35, as follows:

GCACAGCAAGTGTAGACAGGCA
[SEQ ID
No: 35]

Thus, preferably the at least one miRNA target site present in the first nucleic acid sequence comprises a miR-214-5p target site which comprises or consists of a sequence as substantially set out in SEQ ID No: 35, or a variant or fragment thereof.

Preferably, the at least one miRNA target site present in the first nucleic acid sequence comprises a miR-1185-5p target site, which is provided herein as SEQ ID No: 36, as follows:

AACATACAAAGGGTATCCTCT
[SEQ ID N
o: 36]

Thus, preferably the at least one miRNA target site present in the first nucleic acid sequence comprises a miR-1185-5p target site which comprises or consists of a sequence as substantially set out in SEQ ID No: 36, or a variant or fragment thereof.

Preferably, the at least one miRNA target site present in the first nucleic acid sequence comprises a miR-483-5p target site, which is provided herein as SEQ ID No: 37, as follows:

CTCCCTTCTTTCCTCCCGTCTT
[SEQ ID
No: 37]

Thus, preferably the at least one miRNA target site present in the first nucleic acid sequence comprises a miR-483-5p target site which comprises or consists of a sequence as substantially set out in SEQ ID No: 37, or a variant or fragment thereof.

Preferably, the at least one miRNA target site present in the first nucleic acid sequence comprises a miR-365a-3p target site, which is provided herein as SEQ ID No: 38, as follows:

CACATCTGCCCCCAAAAGTCCCT
[SEQ ID
No: 38]

Thus, preferably the at least one miRNA target site present in the first nucleic acid sequence comprises a miR-365a-3p target site which comprises or consists of a sequence as substantially set out in SEQ ID No: 38, or a variant or fragment thereof.

Preferably, the at least one miRNA target site present in the first nucleic acid sequence comprises a miR-127-3p target site, which is provided herein as SEQ ID No: 39, as follows:

AGCCAAGCTCAGACGGATCCGA
[SEQ ID
No: 39]

Thus, preferably the at least one miRNA target site present in the first nucleic acid sequence comprises a miR-127-3p target site which comprises or consists of a sequence as substantially set out in SEQ ID No: 39, or a variant or fragment thereof.

Preferably, the at least one miRNA target site present in the first nucleic acid sequence comprises a miR-574-3p target site, which is provided herein as SEQ ID No: 40, as follows:

TGTGGGTGTGTGCATGAGCGTG
[SEQ ID
No: 40]

Thus, preferably the at least one miRNA target site present in the first nucleic acid sequence comprises a miR-574-3p target site which comprises or consists of a sequence as substantially set out in SEQ ID No: 40, or a variant or fragment thereof.

Preferably, the at least one miRNA target site present in the first nucleic acid sequence comprises a miR- 125b-5p target site, which is provided herein as SEQ ID No: 41, as follows:

TCACAAGTTAGGGTCTCAGGGA
[SEQ ID
No: 41]

Thus, preferably the at least one miRNA target site present in the first nucleic acid sequence comprises a miR- 125b-5p target site which comprises or consists of a sequence as substantially set out in SEQ ID No: 41, or a variant or fragment thereof.

Preferably, the at least one miRNA target site present in the first nucleic acid sequence comprises SEQ ID Nos: 1, 2, 28, 29, 30, 31, 32, 33, 34, 35, 36, 37, 38, 39, 40, and/or 41 or a variant or fragment thereof. In some embodiments, the first nucleic acid sequence may comprise additional miRNA target sites and/or more than one copy of each miRNA target site.

In one embodiment, miRNA29a is represented by miR ID No: MIMAT0000086. The miRNA29a sequence may be provided herein as SEQ ID No: 6, as follows:

UAGCACCAUCUGAAAUCGGUUA
[S
EQ ID No: 6]

Accordingly, preferably miRNA29a comprises or consists of a sequence as substantially set out in SEQ ID No: 6, or a variant or fragment thereof.

In one embodiment, miRNA149 is represented by miR ID No: MIMAT0000450. The miRNA149 sequence may be provided herein as SEQ ID No: 7, as follows:

UCUGGCUCCGUGUCUUCACUCCC
[
SEQ ID No: 7]

Accordingly, preferably miRNA149 comprises or consists of a sequence as substantially set out in SEQ ID No: 7, or a variant or fragment thereof.

In one embodiment, miR-539-5p is represented by miR ID No: MIMAT0003163. The miR-539-5p sequence may be provided herein as SEQ ID No: 42, as follows:

GGAGAAAUUAUCCUUGGUGUGU
[SE
Q ID No: 42]

Accordingly, preferably miR-539-5p comprises or consists of a sequence as substantially set out in SEQ ID No: 42, or a variant or fragment thereof. In one embodiment, miR-487a-3p is represented by miR ID No: MIMAT0002178. The miR-487a-3p sequence may be provided herein as SEQ ID No: 43, as follows:

AAUCAUACAGGGACAUCCAGUU
[SE
Q ID No: 43]

Accordingly, preferably miR-487a-3p comprises or consists of a sequence as substantially set out in SEQ ID No: 43, or a variant or fragment thereof.

In one embodiment, miR-655-3p is represented by miR ID No: MIMAT0003331. The miR-655-3p sequence may be provided herein as SEQ ID No: 44, as follows:

AUAAUACAUGGUUAACCUCUUU
[SE
Q ID No: 44]

Accordingly, preferably miR-655-3p comprises or consists of a sequence as substantially set out in SEQ ID No: 44, or a variant or fragment thereof.

In one embodiment, miR-411-3p is represented by miR ID No: MIMAT0004813. The miR-411-3p sequence may be provided herein as SEQ ID No: 45, as follows:

UAUGUAACACGGUCCACUAACC
[SE
Q ID No: 45]

Accordingly, preferably miR-411-3p comprises or consists of a sequence as substantially set out in SEQ ID No: 45, or a variant or fragment thereof.

In one embodiment, miR-377-5p is represented by miR ID No: MIMAT0004689. The miR-377-5p sequence may be provided herein as SEQ ID No: 46, as follows:

AGAGGUUGCCCUUGGUGAAUUC
[SEQ ID
No: 46]

Accordingly, preferably miR-377-5p comprises or consists of a sequence as substantially set out in SEQ ID No: 46, or a variant or fragment thereof.

In one embodiment, miR-337-5p is represented by miR ID No: MIMAT0004695. The miR-337-5p sequence may be provided herein as SEQ ID No: 47, as follows:

GAACGGCUUCAUACAGGAGUU
[SEQ ID N
o: 47]

Accordingly, preferably miR-337-5p comprises or consists of a sequence as substantially set out in SEQ ID No: 47, or a variant or fragment thereof.

In one embodiment, miR-31-3p is represented by miR ID No: MIMAT0004504. The miR-31-3p sequence may be provided herein as SEQ ID No: 48, as follows:

UGCUAUGCCAACAUAUUGCCAU
[SEQ ID
No: 48]

Accordingly, preferably miR-31-3p comprises or consists of a sequence as substantially set out in SEQ ID No: 48, or a variant or fragment thereof.

In one embodiment, miR-214-5p is represented by miR ID No: MIMAT0004564. The miR-214-5p sequence may be provided herein as SEQ ID No: 49, as follows:

UGCCUGUCUACACUUGCUGUGC
[SEQ ID
No: 49]

Accordingly, preferably miR-214-5p comprises or consists of a sequence as substantially set out in SEQ ID No: 49, or a variant or fragment thereof.

In one embodiment, miR-1185-5p is represented by miR ID No: MIMAT0005798. The miR-1185-5p sequence may be provided herein as SEQ ID No: 50, as follows:

AGAGGAUACCCUUUGUAUGUU
[SEQ ID N
o: 50]

Accordingly, preferably miR-1185-5p comprises or consists of a sequence as substantially set out in SEQ ID No: 50, or a variant or fragment thereof.

In one embodiment, miR-483-5p is represented by miR ID No: MIMAT0004761. The miR-483-5p sequence may be provided herein as SEQ ID No: 51, as follows:

AAGACGGGAGGAAAGAAGGGAG
[SEQ ID
No: 51]

Accordingly, preferably miR-483-5p comprises or consists of a sequence as substantially set out in SEQ ID No: 51, or a variant or fragment thereof.

In one embodiment, miR-365a-3p is represented by miR ID No: MIMAT0009199. The miR-365a-3p sequence may be provided herein as SEQ ID No: 52, as follows:

AGGGACUUUUGGGGGCAGAUGUG
[SEQ ID
No: 52]

Accordingly, preferably miR-365a-3p comprises or consists of a sequence as substantially set out in SEQ ID No: 52, or a variant or fragment thereof.

In one embodiment, miR-127-3p is represented by miR ID No: MIMAT0000446. The miR-127-3p sequence may be provided herein as SEQ ID No: 53, as follows:

UCGGAUCCGUCUGAGCUUGGCU
[SEQ ID
No: 53]

Accordingly, preferably miR-127-3p comprises or consists of a sequence as substantially set out in SEQ ID No: 53, or a variant or fragment thereof.

In one embodiment, miR-574-3p is represented by miR ID No: MIMAT0003239. The miR-574-3p sequence may be provided herein as SEQ ID No: 54, as follows:

CACGCUCAUGCACACACCCACA
[SEQ ID
No: 54]

Accordingly, preferably miR-574-3p comprises or consists of a sequence as substantially set out in SEQ ID No: 54, or a variant or fragment thereof.

In one embodiment, miR-125b-5p is represented by miR ID No: MIMAT0000423. The miR-125b-5p sequence may be provided herein as SEQ ID No: 55, as follows:

UCCCUGAGACCCUAACUUGUGA
[SEQ ID
No: 55]

Accordingly, preferably miR-125b-5p comprises or consists of a sequence as substantially set out in SEQ ID No: 55, or a variant or fragment thereof.

Preferably, miRNAs comprising or consisting of SEQ ID No: 6, 7, 42, 43, 44, 45, 46, 47, 48, 49, 50, 51, 52, 53, 54 and/or 55 or a variant or fragment thereof, target the at least one miRNA target site in the first nucleic acid sequence, and preferably two different miRNA target site in the first nucleic acid sequence.

It will be appreciated that the first nucleic acid sequence may comprise more than one species of miRNA target sequence.

Preferably, the second nucleic acid sequence comprises at least one miRNA target site (or at least one species of miRNA target site). Preferably, the second nucleic acid sequence comprises at least two miRNA target sites (or at least two species of miRNA target site). Preferably, the second nucleic acid sequence comprises at least three miRNA target sites (or at least three species of miRNA target site). Preferably, the second nucleic acid sequence comprises at least four miRNA target sites (or at least four species of miRNA target site. Preferably, the second nucleic acid sequence comprises at least five miRNA target sites (or at least five species of miRNA target site). Preferably, the miRNA targets sites present in the second nucleic acid are target sites for different miRNAs. The more miRNA target sites that are present in the second nucleic acid sequence, then the more tightly regulated gene expression will be. However, it will be appreciated that, for the construct to function adequately, a minimum of only one miRNA target site in each of the first and second nucleic acid sequences is required, provided that they are target sites for different miRNAs.

It will be appreciated that there may be more than one copy of each miRNA target site, i.e. each miRNA target site species may comprise at least one duplication of the target site. Accordingly, preferably, there is at least one copy of each miRNA target site, i.e. each species. Preferably, there is at least two copies of each miRNA target site, i.e. each species. Preferably, there is at least three copies of each miRNA target site, i.e. each species. Preferably, there is at least four copies of each miRNA target site, i.e. each species. Preferably, there is at least five copies of each miRNA target site, i.e. each species.

Preferably, the at least one miRNA target site present in the second nucleic acid sequence is a target site for a miRNA that is expressed in a diseased cell. Preferably, the at least one miRNA target site present in the second nucleic acid is a target site for a miRNA, which is specifically expressed in diseased cells. Preferably, the at least one miRNA target site present in the second nucleic acid is a target site for a miRNA, which is not expressed in a healthy cell, or to very low or undetectable levels. Preferably, the at least one miRNA target site present in the second nucleic acid is a target site for a miRNA, which is ubiquitously expressed in diseased cells.

Up-regulated or increased miRNA expression in a diseased cell may be considered to be at least 5%, 10%, 15% or 20% more than occurs in a healthy cell. Preferably, up-regulated or increased miRNA expression in a diseased cell may be considered to be at least 25%, 30%, 35% or 40% more than occurs in a healthy cell. Preferably, up-regulated or increased miRNA expression in a diseased cell may be considered to be at least 50%, 55%, 60% or 65% more than occurs in a healthy cell. Preferably, up-regulated or increased miRNA expression in a diseased cell may be considered to be at least 70%, 75%, 80% or 85% more than occurs in a healthy cell. Preferably, up-regulated or increased miRNA expression in a diseased cell may be considered to be at least 90%, 95%, 96%, 97%, 98%, 99% or 100 % more than occurs in a healthy cell.

Preferably, the diseased cell is a cancer cell. More preferably, the diseased cell is a T-cell acute lymphoblastic leukaemia (T-ALL) cell.

Preferably, the at least one miRNA target site present in the second nucleic acid comprises a miRNA153, a miR128a, a miR-3687, a miR-92a-2-5p, a miR-20b-3p, a miR-6087, a miR-106a-3p, a miR-7704, a miR-5701, a miR-766-5p, a miR-3609, a miR-3615, and/or a miR-4746-5p target site.

Preferably, the at least one miRNA target site present in the second nucleic acid comprises a miRNA153 target site, which is provided herein as SEQ ID No: 3, as follows:

AGCTGCAACATCACAAAATGA
[SEQ ID N
o: 3]

Thus, preferably the at least one miRNA target site present in the second nucleic acid sequence comprises a miRNA153 target site which comprises or consists of a sequence as substantially set out in SEQ ID No: 3, or a variant or fragment thereof.

Preferably, the at least one miRNA target site present in the second nucleic acid comprises a miR128a target site, which is provided herein as SEQ ID No: 4, as follows:

AAAGAGACCGGTTCACTGTGA
[SEQ ID N
o: 4]

Thus, preferably the at least one miRNA target site present in the second nucleic acid sequence comprises a miR128a target site which comprises or consists of a sequence as substantially set out in SEQ ID No: 4, or a variant or fragment thereof.

Preferably, the at least one miRNA target site present in the second nucleic acid comprises a miR-3687 target site, which is provided herein as SEQ ID No: 56, as follows:

ACGTCGCACGAACGCCTGTCCGGG
[SEQ I
D No: 56]

Thus, preferably the at least one miRNA target site present in the second nucleic acid sequence comprises a miR-3687 target site which comprises or consists of a sequence as substantially set out in SEQ ID No: 56, or a variant or fragment thereof.

Preferably, the at least one miRNA target site present in the second nucleic acid comprises a miR-92a-2-5p target site, which is provided herein as SEQ ID No: 57, as follows:

GTAATGCAACAAATCCCCACCC
[SEQ ID
No: 57]

Thus, preferably the at least one miRNA target site present in the second nucleic acid sequence comprises a miR-92a-2-5p target site which comprises or consists of a sequence as substantially set out in SEQ ID No: 57, or a variant or fragment thereof.

Preferably, the at least one miRNA target site present in the second nucleic acid comprises a miR-20b-3p target site, which is provided herein as SEQ ID No: 58, as follows:

CTGGAAGTGCCCATACTACAGT
[SEQ ID
No: 58]

Thus, preferably the at least one miRNA target site present in the second nucleic acid sequence comprises a miR-20b-3p target site which comprises or consists of a sequence as substantially set out in SEQ ID No: 58, or a variant or fragment thereof.

Preferably, the at least one miRNA target site present in the second nucleic acid comprises a miR-6087 target site, which is provided herein as SEQ ID No: 59, as follows:

GCTCGCCCCCCCGCCTCA
[SEQ ID No:
59]

Thus, preferably the at least one miRNA target site present in the second nucleic acid sequence comprises a miR-6087 target site which comprises or consists of a sequence as substantially set out in SEQ ID No: 59, or a variant or fragment thereof.

Preferably, the at least one miRNA target site present in the second nucleic acid comprises a miR-106a-3p target site, which is provided herein as SEQ ID No: 60, as follows:

GTAAGAAGTGCTTACATTGCAG
[SEQ ID
No: 60]

Thus, preferably the at least one miRNA target site present in the second nucleic acid sequence comprises a miR-106a-3p target site which comprises or consists of a sequence as substantially set out in SEQ ID No: 60, or a variant or fragment thereof.

Preferably, the at least one miRNA target site present in the second nucleic acid comprises a miR-7704 target site, which is provided herein as SEQ ID No: 61, as follows:

CACGTCGCCGCCGACCCCG
[SEQ ID No:
61]

Thus, preferably the at least one miRNA target site present in the second nucleic acid sequence comprises a miR-7704 target site which comprises or consists of a sequence as substantially set out in SEQ ID No: 61, or a variant or fragment thereof.

Preferably, the at least one miRNA target site present in the second nucleic acid comprises a miR-5701 target site, which is provided herein as SEQ ID No: 62, as follows:

AATCAGAACGTGACAATAA
[SEQ ID No:
62]

Thus, preferably the at least one miRNA target site present in the second nucleic acid sequence comprises a miR-5701 target site which comprises or consists of a sequence as substantially set out in SEQ ID No: 62, or a variant or fragment thereof.

Preferably, the at least one miRNA target site present in the second nucleic acid comprises a miR-766-5p target site, which is provided herein as SEQ ID No: 63, as follows:

AAGACCAGCACCAATTCCTCCT
[SEQ ID
No: 63]

Thus, preferably the at least one miRNA target site present in the second nucleic acid sequence comprises a miR-766-5p target site which comprises or consists of a sequence as substantially set out in SEQ ID No: 63, or a variant or fragment thereof.

Preferably, the at least one miRNA target site present in the second nucleic acid comprises a miR-3609 target site, which is provided herein as SEQ ID No: 64, as follows:

CAGCCAGTATTACTCATCACTTTG
[SEQ I
D No: 64]

Thus, preferably the at least one miRNA target site present in the second nucleic acid sequence comprises a miR-3609 target site which comprises or consists of a sequence as substantially set out in SEQ ID No: 64, or a variant or fragment thereof.

Preferably, the at least one miRNA target site present in the second nucleic acid comprises a miR-3615 target site, which is provided herein as SEQ ID No: 65, as follows:

GAGCCGCGAGGAGCCGAGAGA
[SEQ ID N
o: 65]

Thus, preferably the at least one miRNA target site present in the second nucleic acid sequence comprises a miR-3615 target site which comprises or consists of a sequence as substantially set out in SEQ ID No: 65, or a variant or fragment thereof.

Preferably, the at least one miRNA target site present in the second nucleic acid comprises a miR-4746-5p target site, which is provided herein as SEQ ID No: 66, as follows:

TCTGCAGGTTCTCCTGGGACCGG
[SEQ ID
No: 66]

Thus, preferably the at least one miRNA target site present in the second nucleic acid sequence comprises a miR-4746-5p target site which comprises or consists of a sequence as substantially set out in SEQ ID No: 66, or a variant or fragment thereof.

Preferably, the at least one miRNA target site present in the second nucleic acid sequence comprises SEQ ID Nos: 3, 4, 56, 57, 58, 59, 60, 61, 62, 63, 64, 65 and/or 66 or a variant or fragment thereof. In some embodiments, the second nucleic acid sequence may comprise additional miRNA target sites.

In one embodiment, miRNA153 is represented by miR ID No: MIMAT0026480. The miRNA153 sequence may be provided herein as SEQ ID No: 8, as follows:

UCAUUUUUGUGAUGUUGCAGCU
[S
EQ ID No: 8]

Accordingly, preferably miRNA153 comprises or consists of a sequence as substantially set out in SEQ ID No: 8, or a variant or fragment thereof.

In one embodiment, miR128a is represented by miR ID No: MIMAT0000424. The miR128a sequence may be provided herein as SEQ ID No: 9, as follows:

UCACAGUGAACCGGUCUCUUU
[SE
Q ID No: 9]

Accordingly, preferably miR128a comprises or consists of a sequence as substantially set out in SEQ ID No: 9, or a variant or fragment thereof.

In one embodiment, miR-3687 is represented by miR ID No: MIMAT0018115. The miR-3687 sequence may be provided herein as SEQ ID No: 67, as follows:

CCCGGACAGGCGUUCGUGCGACGU
[
SEQ ID No: 67]

Accordingly, preferably miR-3687 comprises or consists of a sequence as substantially set out in SEQ ID No: 67, or a variant or fragment thereof.

In one embodiment miR-92a-2-5p is represented by miR ID No: MIMAT0004508. The miR-92a-2-5p sequence may be provided herein as SEQ ID No: 68, as follows:

GGGUGGGGAUUUGUUGCAUUAC
[SE
Q ID No: 68]

Accordingly, preferably miR-92a-2-5p comprises or consists of a sequence as substantially set out in SEQ ID No: 68, or a variant or fragment thereof.

In one embodiment, miR-20b-3p is represented by miR ID No: MIMAT0004752. The miR-20b-3p sequence may be provided herein as SEQ ID No: 69, as follows:

ACUGUAGUAUGGGCACUUCCAG
[SE
Q ID No: 69]

Accordingly, preferably miR-20b-3p comprises or consists of a sequence as substantially set out in SEQ ID No: 69, or a variant or fragment thereof.

In one embodiment, miR-6087 is represented by miR ID No: MIMAT0023712. The miR-6087 sequence may be provided herein as SEQ ID No: 70, as follows:

UGAGGCGGGGGGGCGAGC
[SEQ ID
No: 70]

Accordingly, preferably miR-6087 comprises or consists of a sequence as substantially set out in SEQ ID No: 70, or a variant or fragment thereof.

In one embodiment, miR-106a-3p is represented by miR ID No: MIMAT0004517. The miR-106a-3p sequence may be provided herein as SEQ ID No: 71, as follows:

CUGCAAUGUAAGCACUUCUUAC
[SE
Q ID No: 71]

Accordingly, preferably miR-106a-3p comprises or consists of a sequence as substantially set out in SEQ ID No: 71, or a variant or fragment thereof.

In one embodiment, miR-7704 is represented by miR ID No: MIMAT0030019. The miR-7704 sequence may be provided herein as SEQ ID No: 72, as follows:

CGGGGUCGGCGGCGACGUG
[SEQ I
D No: 72]

Accordingly, preferably miR-7704 comprises or consists of a sequence as substantially set out in SEQ ID No: 72, or a variant or fragment thereof.

In one embodiment, miR-5701 is represented by miR ID No: MIMAT0022494. The miR-5701 sequence may be provided herein as SEQ ID No: 73, as follows:

UUAUUGUCACGUUCUGAUU
[SEQ I
D No: 73]

Accordingly, preferably miR-5701 comprises or consists of a sequence as substantially set out in SEQ ID No: 73, or a variant or fragment thereof.

In one embodiment, miR-766-5p is represented by miR ID No: MIMAT0022714. The miR-766-5p sequence may be provided herein as SEQ ID No: 74, as follows:

AGGAGGAAUUGGUGCUGGUCUU
[SE
Q ID No: 74]

Accordingly, preferably miR-766-5p comprises or consists of a sequence as substantially set out in SEQ ID No: 74, or a variant or fragment thereof.

In one embodiment, miR-3609 is represented by miR ID No: MIMAT0017986. The miR-3609 sequence may be provided herein as SEQ ID No: 75, as follows:

CAAAGUGAUGAGUAAUACUGGCUG
[
SEQ ID No: 75]

Accordingly, preferably miR-3609 comprises or consists of a sequence as substantially set out in SEQ ID No: 75, or a variant or fragment thereof.

In one embodiment, miR-3615 is represented by miR ID No: MIMAT0017994. The miR-3615 sequence may be provided herein as SEQ ID No: 76, as follows:

UCUCUCGGCUCCUCGCGGCUC
[SEQ
ID No: 76]

Accordingly, preferably miR-3615 comprises or consists of a sequence as substantially set out in SEQ ID No: 76, or a variant or fragment thereof.

In one embodiment, miR-4746-5p is represented by miR ID No: MIMAT0019880. The miR-4746-5p sequence may be provided herein as SEQ ID No: 77, as follows:

CCGGUCCCAGGAGAACCUGCAGA
[S
EQ ID No: 77]

Accordingly, preferably miR-4746-5p comprises or consists of a sequence as substantially set out in SEQ ID No: 77, or a variant or fragment thereof.

Preferably, miRNAs comprising or consisting of SEQ ID No: 8, 9, 67, 68, 69, 70, 71, 72, 73, 74, 75, 76 and/or 77 or a variant or fragment thereof, target the at least one miRNA target site in the second nucleic acid sequence, and preferably two different miRNA target site in the second nucleic acid sequence.

Advantageously, therefore, the delivery of the therapeutic molecule or reporter molecule is regulated both by microRNAs present only (or highly expressed) in a patient’s T-ALL cells (e.g. miRNA153, miRNA128a, miR-3687, miR-92a-2-5p, miR-20b-3p, miR-6087, miR-106a-3p, miR-7704, miR-5701, miR-766-5p, miR-3609, miR-3615, and/or miR-4746-5p) and microRNAs specifically absent (or significantly less expressed) in those cells (e.g. miRNA29a, miRNA149, miR-539-5p, miR-487a-3p, miR-655-3p, miR-411-3p, miR-377-5p, miR-337-5p, miR-31-3p, miR-214-5p, miR-1185-5p, miR-483-5p, miR-365a-3p, miR-127-3p, miR-574-3p, and/or miR-125b-5p). The construct of the invention uses the concomitant presence and absence of specific microRNAs to positively regulate the expression of the therapeutic molecule or reporter molecule. Preferably, and advantageously, this dual layer of regulation is achieved by the use of a bidirectional expression vector. It will be appreciated that a negative feedback loop through gene expression provides additional control.

The first promoter may be any suitable promoter, including a constitutive promoter, an activatable promoter, an inducible promoter, or a tissue-specific promoter. The first promoter may be a ubiquitously expressed promoter, or a cancer cell-specific promoter. Preferably, the first promoter is a ubiquitously expressed promoter.

The first promoter may be selected from the group consisting of elongation factor 1 alpha (EF1α) promoter, EF1α short (EFS) promoter, phosphoglycerate kinase (PGK) promoter, cytomegalovirus (CMV) immediate early promoter, spleen focus forming virus (SFFV) promoter, CASI promoter, myeloproliferative sarcoma virus (MPSV) long terminal repeat, murine stem cell virus (MSCV) long terminal repeat (LTR), and the composite CAG promoter, (consisting of the CMV immediate early enhancer and the chicken β-actin promoter).

Preferably, the first promoter extends in a 5′ to 3′ direction.

The first nucleic acid coding sequence may encode a reporter molecule or a therapeutic molecule.

The reporter molecule may be an optical reporter, a nuclear medicine reporter or an MRI reporter.

The optical reporter may be a fluorescent protein or luciferase. The nuclear medicine reporter may be herpes simplex virus type 1 [HSV1] thymidine kinase (TK), human mitochondrial TK type 2, dopaminergic receptor, dopamine 2 receptor, sodium iodide symporter, norepinephrine transporter, Somatostatin receptor or oestrogen receptor. The MRI reporter may be transferrin receptor, β-galactosidase, tyrosinase, ferritin or lysine-rich protein (LRP).

The therapeutically active molecule may be a therapeutic protein and/or a nucleic acid.

The nucleic acid may be a DNA, RNA or a chimeric DNA/RNA molecule. The nucleic acid may be a gene-silencing molecule, examples include an RNAi molecule, including siNA, siRNA, shRNA, miRNA, ribozymes and antisense molecules. The nucleic acid may be a guide RNA.

The skilled person would understand that the term guide RNA refers to the non-coding RNA component of the CRISPR/Cas system, which binds to complementary target DNA sequences. Guide RNA first binds to a Cas enzyme and the gRNA sequence guides the complex via pairing to a specific location on the DNA, where Cas performs its endonuclease activity by cutting the target DNA strand.

Gene-silencing molecules may be antisense molecules (antisense DNA or antisense RNA) or ribozyme molecules. Ribozymes and antisense molecules may be used to inhibit the transcription of essential genes in the diseased cell. Antisense molecules are oligonucleotides that bind in a sequence-specific manner to nucleic acids, such as DNA or RNA. When bound to mRNA that has a complimentary sequence, antisense RNA prevents translation of the mRNA. Triplex molecules refer to single antisense DNA strands that bind duplex DNA forming a colinear triplex molecule, thereby preventing transcription.

The therapeutically active molecule may be therapeutic protein, wherein the first nucleic acid encodes an mRNA molecule that encodes the therapeutic protein.

The therapeutic protein may be a recombinant protein having therapeutic applications.

The therapeutic protein may be an endonuclease. For example, therapeutic protein may be a CRISPR endonuclease, such as a CRISPR-associated protein (Cas). Preferably, the Cas protein may be Cas9 and/or Cpf1.

The therapeutic molecule may comprise both components of the CRIPSR/Cas system, including a Cas protein and a guide RNA. In this embodiment, the construct may comprise an additional promoter for driving expression of the Cas protein.

The therapeutic protein may be a CAR (Chimeric Antigen Receptor). For example, therapeutic protein may be a CAR protein that enables the setup of a CAR-T cell for in vivo T-cell immunotherapy.

The therapeutic protein may be an effector protein. Preferably, the therapeutic protein may be capable of triggering the apoptosis cascade within a tumour cell. Thus, preferably the therapeutic protein may be an apoptosis driver protein. Preferably, the apoptosis driver protein may be Bax, Apoptin,E4orf4 and/or Bim.

Preferably, the apoptosis driver protein may be Bax, which may be represented by Gene ID No: 581 which is provided herein as SEQ ID No: 16, as follows:

MDGSGEQPRGGGPTSSEQIMKTGALLLQGFIQDRAGRMGGEAPELALDPV
PQDASTKKLSECLKRIGDELDSNMELQRMIAAVDTDSPREVFFRVAADMF
SDGNFNWGRVVALFYFASKLVLKALCTKVPELIRTIMGWTLDFLRERLLG
WIQDQGGWGLPLAESLKRLMSLSPGRPPLLLWDAHVADRDHLCGGSAHRL
THHLEEDGLRPPAALDCVFPP
[SEQ ID N
o: 16]

Accordingly, preferably Bax comprises or consists of an amino acid sequence as substantially set out in SEQ ID No: 16, or a biologically active variant or fragment thereof.

In one embodiment, Bax may be encoded by a nucleotide sequence which is provided herein as SEQ ID No: 17, as follows:

gcgctgcggc cgcccgcgcg gacccggcga gaggcggcgg cgggagcggc ggtgatggacgggtccggggagcagcccag aggcgggggg cccaccagct
ctgagcagat catgaagacatgcttcaggg tttcatccag gatcgagcag ggcgaatggg gggggaggcacccgagctgg ccctggaccc ggtgcctcag
gatgcgtcca ccaagaagct gagcgagtgtctcaagcgca tcggggacga actggacagt aacatggagc tgcagaggat gattgccgccgtggacacag
actccccccg agaggtcttt ttccgagtgg cagctgacat gttttctgacggcaacttca actggggccg ggttgtcgcc cttttctact ttgccagcaa
actggtgctcaaggccctgt gcaccaaggt gccggaactg atcagaacca tcatgggctg gacattggacttcctccggg agcggctgtt gggctggatc
caagaccagg gtggttgggg gctgcccctggccgagtcac tgaagcgact gatgtccctg tctccaggac ggcctcctct cctactttgggacgcccacg
tggcagaccg tgaccatctt tgtggcggga gtgctcaccg cctcactcaccatctggaag aagatgggct gaggccccca gctgccttgg actgtgtttt
tcctccataaattatggcat ttttctggga ggggtgggga ttgggggacg tgggcatttt tcttacttttgtaattattg gggggtgtgg ggaagagtgg
tcttgagggg gtaataaacc tccttcgggacaca
[SEQ ID No: 17]

Hence, preferably the Bax polypeptide or a biologically active variant or fragment thereof may be encoded by a nucleotide sequence substantially as set out in SEQ ID NO: 17, or a variant or fragment thereof.

Preferably, the apoptosis driver protein may be Apoptin, which may be represented by Gene ID No: 1494446 which is provided herein as SEQ ID No: 18, as follows:

MNALQEDTPPGPSTVFRPPTSSRPLETPHCREIRIGIAGITITLSLCGCA
NARAPTLRSATADNSESTGFKNVPDLRTDQPKPPSKKRSCDPSEYRVSEL
KESLITTTPSRPRTARRRIRL
[SEQ ID N
o: 18]

Accordingly, preferably Apoptin comprises or consists of an amino acid sequence as substantially set out in SEQ ID No: 18, or a biologically active variant or fragment thereof.

In one embodiment, Apoptin may be encoded by a nucleotide sequence which is provided herein as SEQ ID No: 19, as follows:

atgaacgctc tccaagaaga tactccaccc ggaccatcaa cggtgttcag gccaccaacaagttcacggc cgttggaaac ccctcactgc agagagatcc
ggattggtat cgctggaattacaatcactc tatcgctgtg tggctgcgcg aatgctcgcg ctcccacgct aagatctgcaactgcggaca attcagaaag
cactggtttc aagaatgtgc cggacttgag gaccgatcaacccaagcctc cctegaagaa gcgatcctgc gacccctccg agtacagggt
aagcgagctaaaagaaagct tgattaccac tactcccagc cgaccccgaa ccgcaagaag gcgtataagactgtaa
[SEQ ID No: 19]

Hence, preferably the Apoptin polypeptide or a biologically active variant or fragment thereof may be encoded by a nucleotide sequence substantially as set out in SEQ ID NO: 19, or a variant or fragment thereof.

Preferably, the apoptosis driver protein may be E4orf4, which may be represented by Gene ID No: AC_000007.1, which is provided herein as SEQ ID No: 20, as follows:

MVLPALPAPPVCDSQNECVGWLGVAYSAVVDVIRAAAHEGVYIEPEARGR
LDALREWIYYNYYTERAKRRDRRRRSVCHARTWFCFRKYDYVRRSIWHDT
TTNTISVVSAHSVQ
[SEQ ID No: 20]

Accordingly, preferably E4orf4 comprises or consists of an amino acid sequence as substantially set out in SEQ ID No: 20, or a biologically active variant or fragment thereof.

In one embodiment, E4orf4 may be encoded by a nucleotide sequence which is provided herein as SEQ ID No: 21, as follows:

atggttcttccagctcttcccgctcctcccgtgtgtgactcgcagaacga
atgtgtaggttggctgggtgtggcttattctgcggtggtggatgttatca
gggcagcggcgcatgaaggagtttacatagaacccgaagccagggggcgc
ctggatgctttgagagagtggatatactacaactactacacagagcgagc
taagcgacgagaccggagacgcagatctgtttgtcacgcccgcacctggt
tttgcttcaggaaatatgactacgtccggcgttccatttggcatgacact
acgaccaacacgatctcggttgtctcggcgcactccgtacagtag
[SEQ ID No: 21]

Hence, preferably the E4orf4 polypeptide or a biologically active variant or fragment thereof may be encoded by a nucleotide sequence substantially as set out in SEQ ID NO: 21, or a variant or fragment thereof.

Preferably, the apoptosis driver protein may be Bim (BCL2L11), which may be represented by Gene ID No: 10018 which is provided herein as SEQ ID No: 25, as follows:

MAKQPSDVSSECDREGRQLQPAERPPQLRPGAPTSLQTEPQGNPEGNHGG
EGDSCPHGSPQGPLAPPASPGPFATRSPLFIFMRRSSLLSRSSSGYFSFD
TDRSPAPMSCDKSTQTPSPPCQAFNHYLSAMASMRQAEPADMRPEIWIAQ
ELRRIGDEFNAYYARRVFLNNYQAAEDHPRMVILRLLRYIVRLVWRMH
[SEQ ID No: 25]

Accordingly, preferably Bim comprises or consists of an amino acid sequence as substantially set out in SEQ ID No: 25, or a biologically active variant or fragment thereof.

In one embodiment, Bim may be encoded by a nucleotide sequence which is provided herein as SEQ ID No: 26, as follows:

atggcaaagcaaccttctgatg taagttctga gtgtgaccga gaaggtagac aattgcagcc
tgcggagaggcctccccagc tcagacctgg ggcccctacc tccctacaga cagagccaca
aggtaatcctgaaggcaatc acggaggtga aggggacagc tgcccccacg gcagccctca
gggcccgctggccccacctg ccagccctgg cccttttgct accagatccc cgcttttcat
ctttatgagaagatcctccc tgctgtctcg atcctccagt gggtatttct cttttgacac
agacaggagcccagcaccca tgagttgtga caaatcaaca caaaccccaa gtcctccttg
ccaggccttcaaccactatc tcagtgcaat ggcttccatg aggcaggctg aacctgcaga
tatgcgcccagagatatgga tcgcccaaga gttgcggcgt attggagacg agtttaacgc
ttactatgcaaggagggtat ttttgaataa ttaccaagca gccgaagacc acccacgaat
ggttatcttacgactgttac gttacattgt ccgcctggtg tggagaatgc attga
[SEQ ID No: 26]

Hence, preferably the Bim polypeptide or a biologically active variant or fragment thereof may be encoded by a nucleotide sequence substantially as set out in SEQ ID NO: 26, or a variant or fragment thereof.

The second promoter may be any suitable promoter, including a constitutive promoter, an activatable promoter, an inducible promoter, or a tissue-specific promoter. Preferably, the first promoter is a ubiquitously expressed promoter. Preferably, the second promoter is a different promoter to the first promoter.

The second promoter may be arranged in the same orientation in the construct as the first promoter. The second promoter may extend in a 3′ to 5′ direction. However, preferably the second promoter is arranged in an opposite orientation in the construct to the first promoter. Preferably, the second promoter extends in a 3′ to 5′ direction. Preferably, the promoters are bidirectional. The inventor’s use of bidirectional promoters provides the additional advantage of coordinated expression of two genes without the need of either additional genetic elements such as IRES elements, fusion proteins or self-cleaving 2A peptides, which can result in weak co-expression of two genes and which require additional transgene engineering that may affect protein stability and/or biological function. In addition, the use of bidirectional promoters avoids unidirectional promoter interference issues that may arise, for example when using lentivectors.

Accordingly, in one embodiment, the first expression cassette is arranged in the same direction as the second expression cassette. However, in a preferred embodiment, the first expression cassette is arranged in the opposite direction to the second expression cassette.

The second promoter may be selected from the group consisting of: elongation factor 1 alpha (EF1α) promoter, EF1α short (EFS) promoter, phosphoglycerate kinase (PGK) promoter, cytomegalovirus (CMV) immediate early promoter, spleen focus forming virus (SFFV) promoter, CASI promoter, myeloproliferative sarcoma virus (MPSV) long terminal repeat, murine stem cell virus (MSCV) long terminal repeat (LTR), and the composite CAG promoter, (consisting of the CMV immediate early enhancer and the chicken β-actin promoter).

In one embodiment, the inhibitor encoded by the second nucleic acid sequence is a direct inhibitor of the therapeutically active molecule or a reporter molecule.

Preferably, however, the inhibitor encoded by the second nucleic acid sequence is an inhibitor of the first promoter. The inhibitor of the first promoter may be part of the Tetracycline-Controlled Operator System or the Cumate-controlled operator system, such systems are well known to those skilled in the art.

The inhibitor of the first promoter may be a Lac operon, wherein the second nucleic acid sequence comprises a Lac repressor and the first promoter comprises a Lac operator regulator site.

In one embodiment, the first promoter and first nucleic acid sequence is 5′ to the second promoter sequence and the second nucleic acid sequence. In one embodiment, the first promoter and first nucleic acid sequence is 3′ to the second promoter sequence and the second nucleic acid sequence.

In one embodiment the genetic construct may comprise, in this specified order in a 5′ to 3′ orientation, the first promoter sequence operably linked to the first nucleic acid sequence, the first nucleic acid sequence encoding a reporter molecule and/or a therapeutic molecule, the second promoter sequence operably linked to the second nucleic acid sequence, the second nucleic acid sequence encoding an inhibitor of the first promoter.

In a preferred embodiment, however, the genetic construct may comprise, in this specified order in a 5′ to 3′ orientation, the second promoter sequence operably linked to the second nucleic acid, the second nucleic acid sequence encoding an inhibitor of the first promoter, the first promoter sequence operably linked to the first nucleic acid sequence and the first nucleic acid sequence encoding a reporter molecule and/or a therapeutic molecule.

The use of 5′ and 3′ indicates that the features are either upstream or downstream, and is not intended to indicate that the features are necessarily terminal features.

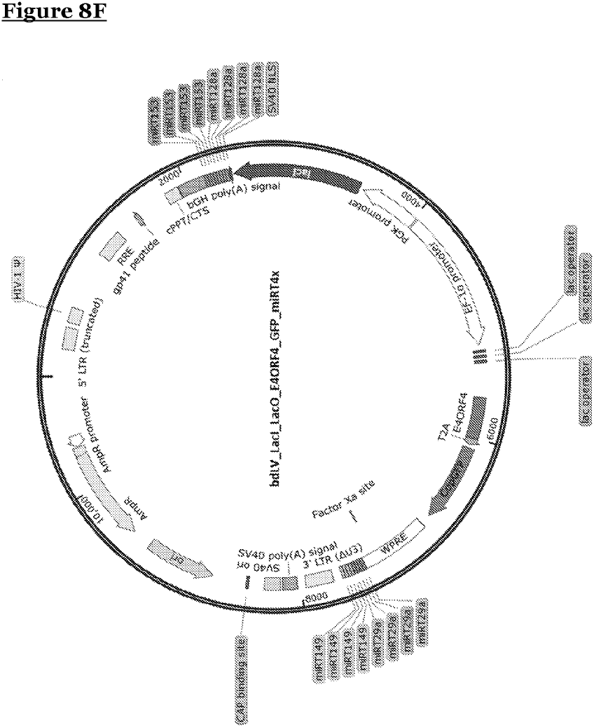

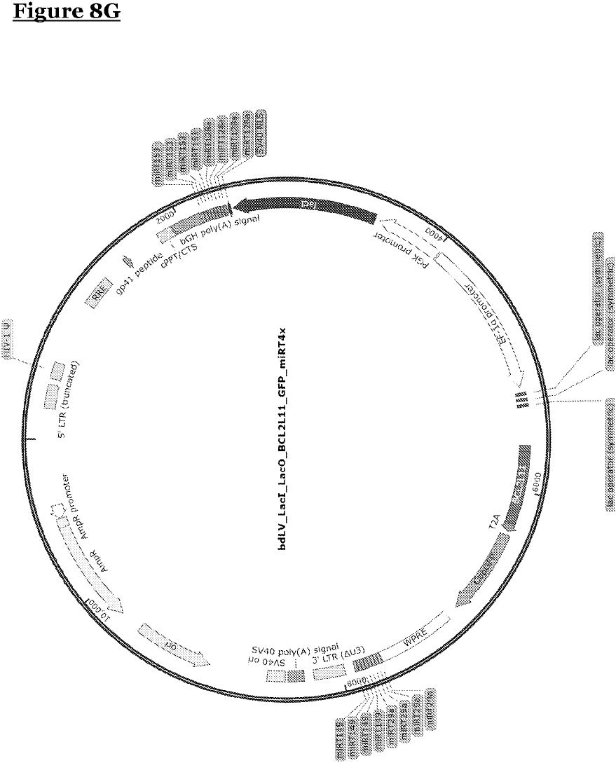

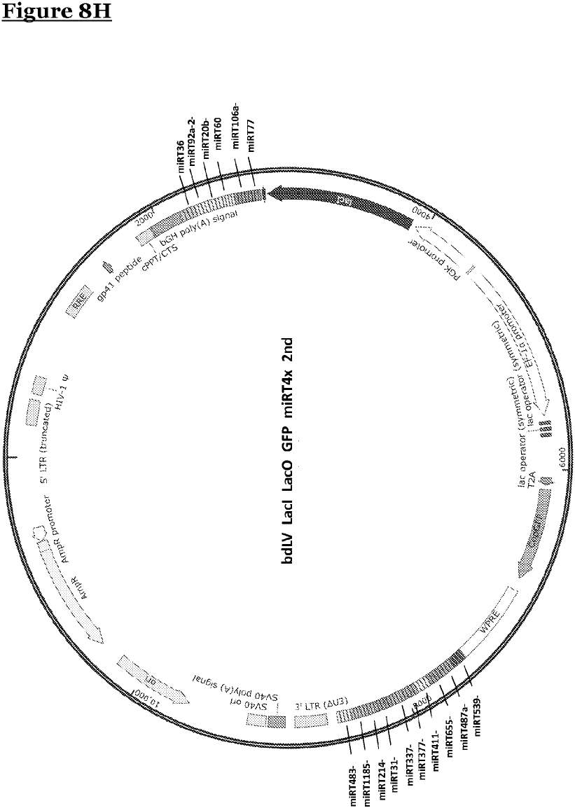

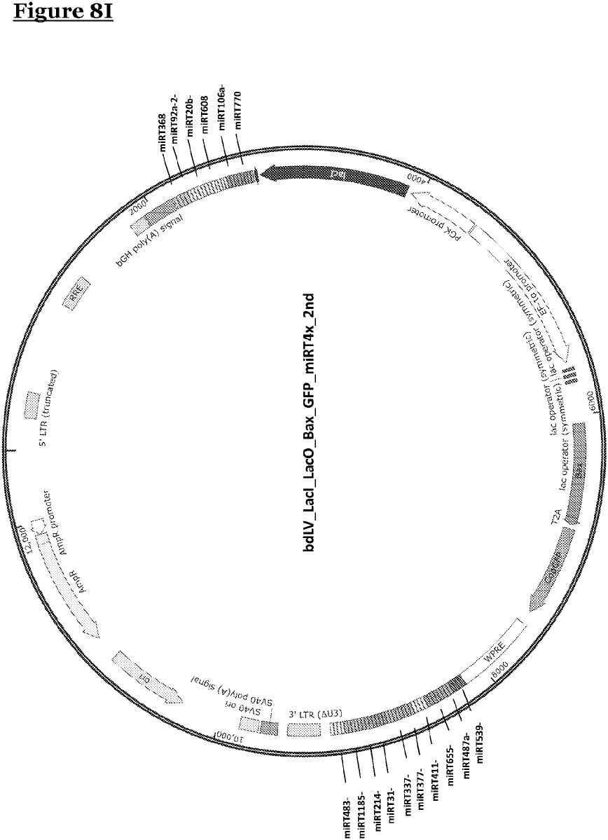

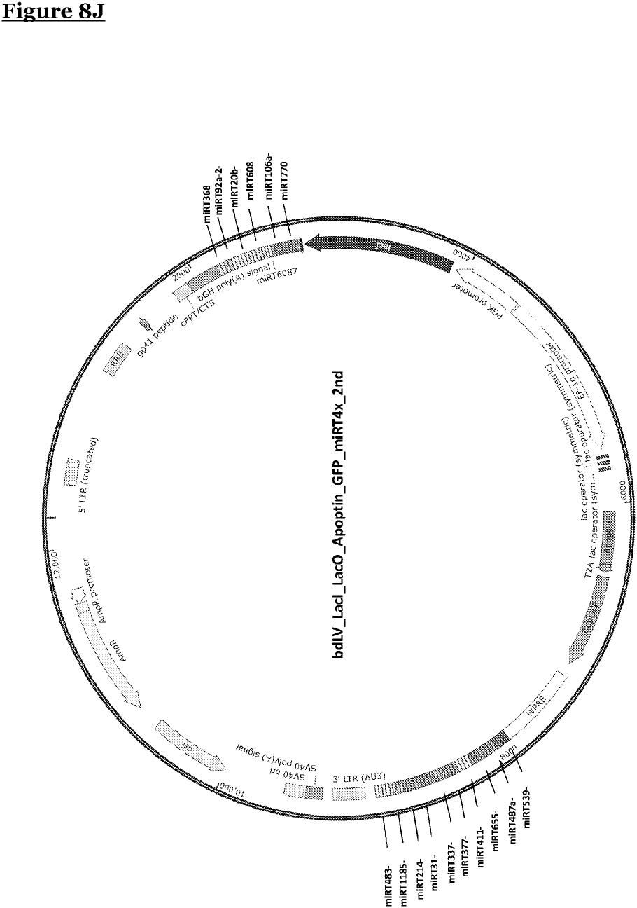

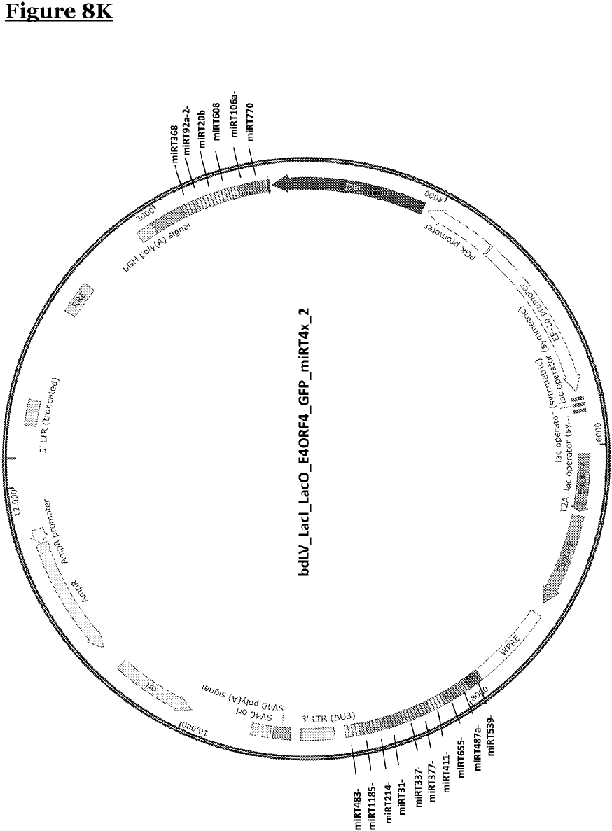

An embodiment of the genetic construct is shown in FIG. 8c, and is referred to herein as SEQ ID No: 5, as follows:

acgcgtgtagtcttatgcaatactcttgtagtcttgcaacatggtaacga
tgagttagcaacatgccttacaaggagagaaaaagcaccgtgcatgccga
ttggtggaagtaaggtggtacgatcgtgccttattaggaaggcaacagac
gggtctgacatggattggacgaaccactgaattgccgcattgcagagata
ttgtatttaagtgcctagctcgatacaataaacgggtctctctggttaga
ccagatctgagcctgggagctctctggctaactagggaacccactgctta
agcctcaataaagcttgccttgagtgcttcaagtagtgtgtgcccgtctg
ttgtgtgactctggtaactagagatccctcagacccttttagtcagtgtg
gaaaatctctagcagtggcgcccgaacagggacctgaaagcgaaagggaa
accagagctctctcgacgcaggactcggcttgctgaagcgcgcacggcaa
gaggcgaggggcggcgactggtgagtacgccaaaaattttgactagcgga
ggctagaaggagagagatgggtgcgagagcgtcagtattaagcgggggag
aattagatcgcgatgggaaaaaattcggttaaggccagggggaaagaaaa
aatataaattaaaacatatagtatgggcaagcagggagctagaacgattc
gcagttaatcctggcctgttagaaacatcagaaggctgtagacaaatact
gggacagctacaaccatcccttcagacaggatcagaagaacttagatcat
tatataatacagtagcaaccctctattgtgtgcatcaaaggatagagata
aaagacaccaaggaagctttagacaagatagaggaagagcaaaacaaaag
taagaccaccgcacagcaagcggccactgatcttcagacctggaggagga
gatatgagggacaattggagaagtgaattatataaatataaagtagtaaa
aattgaaccattaggagtagcacccaccaaggcaaagagaagagtggtgc
agagagaaaaaagagcagtgggaataggagctttgttccttgggttcttg
ggagcagcaggaagcactatgggcgcagcctcaatgacgctgacggtaca
ggccagacaattattgtctggtatagtgcagcagcagaacaatttgctga
gggctattgaggcgcaacagcatctgttgcaactcacagtctggggcatc
aagcagctccaggcaagaatcctggctgtggaaagatacctaaaggatca
acagctcctggggatttggggttgctctggaaaactcatttgcaccactg
ctgtgccttggaatgctagttggagtaataaatctctggaacagattTgg
aatcacacgacctggatggagtgggacagagaaattaacaattacacaag
cttaatacactccttaattgaagaatcgcaaaaccagcaagaaaagaatg
aacaagaattattggaattagataaatgggcaagtttgtggaattggttt
aacataacaaattggctgtggtatataaaattattcataatgatagtagg
aggcttggtaggtttaagaatagtttttgctgtactttctatagtgaata
gagttaggcagggatattcaccattatcgtttcagacccacctcccaacc
ccgaggggacccgacaggcccgaaggaatagaagaagaaggtggagagag
agacagagacagatccattcgattagtgaacggatctcgacggtATCGGT
taacttttaaaagaaaaggggggattggggggtacagtgcaggggaaaga
atagtagacataatagcaacagacatacaaactaaagaattacaaaaaca
aattacaaaaattcaaaattttatGTCGAGccatagagcccaccgcatcc
ccagcatgcctgctattgtcttcccaatcctcccccttgctgtcctgccc
caccccaccccccagaatagaatgacacctactcagacaatgcgatgcaa
tttcctcattttattaggaaaggacagtgggagtggcaccttccagggtc
aaggaaggcacgggggaggggcaaacaacagatggctggcaactagaagg
cacagtcgCtcgagAGTAAAACACTACAACGTCGATAGCAGTAAAACACT
ACAACGTCGACACTAGTAAAACACTACAACGTCGATAGCAGTAAAACACT
ACAACGTCGACACTAGTGTCACTTGGCCAGAGAAATAGCAGTGTCACTTG
GCCAGAGAAACACTAGTGTCACTTGGCCAGAGAAATAGCAGTGTCACTTG
GCCAGAGAAActcgagTCAAACCTTCCTCTTCTTCTTAGGAGGCCTCAGG
CTGCTCTGCCCGCTTTCCAGTCGGGAAACCTGTCGTGCCAGCTGCATTAA
TGAATCGGCCAACGCGCGGGGAGAGGCGGTTTGCGTATTGGGCGCCAGGG
TGGTTTTTCTTTTCACCAGTGAGACGGGCAACAGCTGATTGCCCTTCACC
GCCTGGCCCTGAGAGAGTTGCAGCAAGCGGTCCACGCTGGTTTGCCCCAG
CAGGCGAAAATCCTGTTTGATGGTGGTTAACGGCGGGATATAACATGAGC
TGTCTTCGGTATCGTCGTATCCCACTACCGAGATATCCGCACCAACGCGC
AGCCCGGACTCGGTAATGGCGCGCATTGCGCCCAGCGCCATCTGATCGTT
GGCAACCAGCATCGCAGTGGGAACGATGCCCTCATTCAGCATTTGCATGG
TTTGTTGAAAACCGGACATGGCACTCCAGTCGCCTTCCCGTTCCGCTATC
GGCTGAATTTGATTGCGAGTGAGATATTTATGCCAGCCAGCCAGACGCAG
ACGCGCCGAGACAGAACTTAATGGGCCCGCTAACAGCGCGATTTGCTGGT
GACCCAATGCGACCAGATGCTCCACGCCCAGTCGCGTACCGTCTTCATGG
GAGAAAATAATACTGTTGATGGGTGTCTGGTCAGAGACATCAAGAAATAA
CGCCGGAACATTAGTGCAGGCAGCTTCCACAGCAATGGCATCCTGGTCAT
CCAGCGGATAGTTAATGATCAGCCCACTGACGCGTTGCGCGAGAAGATTG
TGCACCGCCGCTTTACAGGCTTCGACGCCGCTTCGTTCTACCATCGACAC
CACCACGCTGGCACCCAGTTGATCGGCGCGAGATTTAATCGCCGCGACAA
TTTGCGACGGCGCGTGCAGGGCCAGACTGGAGGTGGCAACGCCAATCAGC
AACGACTGTTTGCCCGCCAGTTGTTGTGCCACGCGGTTGGGAATGTAATT
CAGCTCCGCCATCGCCGCTTCCACTTTTTCCCGCGTTTTCGCAGAAACGT
GGCTGGCCTGGTTCACCACGCGGGAAACGGTCTGATAAGAGACACCGGCA
TACTCTGCGACATCGTATAACGTTACTGGTTTCATGGTGGGAGGGTACcT
AGGTAAGCTTGGGCTGCAGGTCGAAAGGCCCGGAGATGAGGAAGAGGAGA
ACAGCGCGGCAGACGTGCGCTTTTGAAGCGTGCAGAATGCCGGGCCTCCG
GAGGACCTTCGGGCGCCCGCCCCGCCCCTGAGCCCGCCCCTGAGCCCGCC
CCCGGACCCACCCCTTCCCAGCCTCTGAGCCCAGAAAGCGAAGGAGCAAA
GCTGCTATTGGCCGCTGCCCCAAAGGCCTACCCGCTTCCATTGCTCAGCG
GTGCTGTCCATCTGCACGAGACTAGTGAGACGTGCTACTTCCATTTGTCA
CGTCCTGCACGACGCGAGCTGCGGGGCGGGGGGGAACTTCCTGACTAGGG
GAGGAGTAGAAGGTGGCGCGAAGGGGCCACCAAAGAACGGAGCCGGTTGG
CGCCTACCGGTGGATGTGGAATGTGTGCGAGGCCAGAGGCCACTTGTGTA
GCGCCAAGTGCCCAGCGGGGCTGCTAAAGCGCATGCTCCAGACTGCCTTG
GGAAAAGCGCCTCCCCTACCCGGTAGAAGCTAGCGGCTCCGGTGCCCGTC
AGTGGGCAGAGCGCACATCGCCCACAGTCCCCGAGAAGTTGTGGGGAGGG
GTCGGCAATTGAACCGGTGCCTAGAGAAGGTGGCGCGGGGTAAACTGGGA
AAGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTTT
GCCGCCAGAACACAGGTAAGTGCCGTGTGTGGTTCCCGCGGGCCTGGCCT
CTTTACGGGTTATGGCCCTTGCGTGCCTTGAATTACTTCCACCTGGCTGC
AGTACGTGATTCTTGATCCCGAGCTTCGGGTTGGAAGTGGGTGGGAGAGT
TCGAGGCCTTGCGCTTAAGGAGCCCCTTCGCCTCGTGCTTGAGTTGAGGC
CTGGCCTGGGCGCTGGGGCCGCCGCGTGCGAATCTGGTGGCACCTTCGCG
CCTGTCTCGCTGCTTTCGATAAGTCTCTAGCCATTTAAAATTTTTGATGA
CCTGCTGCGACGCTTTTTTTCTGGCAAGATAGTCTTGTAAATGCGGGCCA
AGATCTGCACACTGGTATTTCGGTTTTTGGGGCCGCGGGCGGCGACGGGG
CCCGTGCGTCCCAGCGCACATGTTCGGCGAGGCGGGGCCTGCGAGCGCGG
CCACCGAGAATCGGACGGGGGTAGTCTCAAGCTGGCCGGCCTGCTCTGGT
GCCTGGCCTCGCGCCGCCGTGTATCGCCCCGCCCTGGGCGGCAAGGCTGG
CCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGG
TGAGTCACCCACACAAAGGAAAAGGGCCTTTCCGTCCTCAGCCGTCGCTT
CATGTGACTCCACGGAGTACCGGGCGCCGTCCAGGCACCTCGATTAGTTC
TCGACCTTTTGGAGTACGTCGTCTTTAGGTTGGGGGGAGGGGTTTTATGC
GATGGAGTTTCCCCACACTGAGTGGGTGGAGACTGAAGTTAGGCCAGCTT
GGCACTTGATGTAATTCTCCTTGGAATTTGCCCTTTTTGAGTTTGGATCT
TGGTTCATTCTCAAGCCTCAGACAGTGGTTCAAAGTTTTTTTCTTCCATT
TCAGGTGTCGTGAtctagagctagcgAATTCGTAAATATAAAATTTACTA
GGTTGTGGAATTGTGAGCGCTCACAATTCCACAGTCGACCCTAGGTTGTG
GAATTGTGAGCGCTCACAATTCCACAGTCGACCCTAGGTTGTGGAATTGT
GAGCGCTCACAATTCCACAGTCGACCCTAGTGTATAATGTGTTAAACTAC
GGATCCGTCTCCCATTAGGCCTACAATGGTGAGACAAGTAGCCAACAGGG
AAGGGTTGCAAATATCATTTGGGCACACCTATGATAATATTGATGAAGCA
GACAGTATTCAGCAAGTAACTGAGAGGTGGGAAGCTCAAAGCCAAAGTCC
TAATGTGCAGTCAGGTGAATTTATTGAAAAATTTGAGGCTCCTGGTGGTG
CAAATCAAAGAACTGCTCCTCAGGGATCCTAATTGTTTGTGTATTTTAGG
Cggccgctgagggcagaggaagtcttctaacatgcggtgacgtggaggag
aatcccggcccttccggaATGGAGAGCGACGAGAGCGGCCTGCCCGCCAT
GGAGATCGAGTGCCGCATCACCGGCACCCTGAACGGCGTGGAGTTCGAGC
TGGTGGGCGGCGGAGAGGGCACCCCCAAGCAGGGCCGCATGACCAACAAG
ATGAAGAGCACCAAAGGCGCCCTGACCTTCAGCCCCTACCTGCTGAGCCA
CGTGATGGGCTACGGCTTCTACCACTTCGGCACCTACCCCAGCGGCTACG
AGAACCCCTTCCTGCACGCCATCAACAACGGCGGCTACACCAACACCCGC
ATCGAGAAGTACGAGGACGGCGGCGTGCTGCACGTGAGCTTCAGCTACCG
CTACGAGGCCGGCCGCGTGATCGGCGACTTCAAGGTGGTGGGCACCGGCT
TCCCCGAGGACAGCGTGATCTTCACCGACAAGATCATCCGCAGCAACGCC
ACCGTGGAGCACCTGCACCCCATGGGCGATAACGTGCTGGTGGGCAGCTT
CGCCCGCACCTTCAGCCTGCGCGACGGCGGCTACTACAGCTTCGTGGTGG
ACAGCCACATGCACTTCAAGAGCGCCATCCACCCCAGCATCCTGCAGAAC
GGGGGCCCCATGTTCGCCTTCCGCCGCGTGGAGGAGCTGCACAGCAACAC
CGAGCTGGGCATCGTGGAGTACCAGCACGCCTTCAAGACCCCCATCGCCT
TCGCCAGATCCCGCGCTCAGTCGTCCAATTCTGCCGTGGACGGCACCGCC
GGACCCGGCTCCACCGGATCTCGCTAGAGCTGAATCtaagtcgacaatca
acctctggattacaaaatttgtgaaagattgactggtattcttaactatg
ttgctccttttacgctatgtggatacgctgctttaatgcctttgtatcat
gctattgcttcccgtatggctttcattttctcctccttgtataaatcctg
gttgctgtctctttatgaggagttgtggcccgttgtcaggcaacgtggcg
tggtgtgcactgtgtttgctgacgcaacccccactggttggggcattgcc
accacctgtcagctcctttccgggactttcgctttccccctccctattgc
cacggcggaactcatcgccgcctgccttgcccgctgctggacaggggctc
ggctgttgggcactgacaattccgtggtgttgtcggggaaatcatcgtcc
tttccttggctgctcgcctgtgttgccacctggattctgcgcgggacgtc
cttctgctacgtcccttcggccctcaatccagcggaccttccttcccgcg
gcctgctgccggctctgcggcctcttccgcgtcttcgccttcgccctcag
acgagtcggatctccctttgggccgcctccccgcccgggTAACCGATTTC
AGATGGTGCTACGATTAACCGATTTCAGATGGTGCTATCACTAACCGATT
TCAGATGGTGCTACGATTAACCGATTTCAGATGGTGCTATCACGGGAGTG
AAGACACGGAGCCAGACGATGGGAGTGAAGACACGGAGCCAGATCACGGG
AGTGAAGACACGGAGCCAGACGATGGGAGTGAAGACACGGAGCCAGAccc
gggtacctttaagaccaatgacttacaaggcagctgtagatcttagccac
tttttaaaagaaaaggggggactggaagggctaattcactcccaacgaaa
ataagatctgctttttgcttgtactgggtctctctggttagaccagatct
gagcctgggagctctctggctaactagggaacccactgcttaagcctcaa
taaagcttgccttgagtgcttcaagtagtgtgtgcccgtctgttgtgtga
ctctggtaactagagatccctcagacccttttagtcagtgtggaaaatct
ctagcagtagtagttcatgtcatcttattattcagtatttataacttgca
aagaaatgaatatcagagagtgagaggaacttgtttattgcagcttataa
tggttacaaataaagcaatagcatcacaaatttcacaaataaagcatttt
tttcactgcattctagttgtggtttgtccaaactcatcaatgtatcttat
catgtctggctctagctatcccgcccctaactccgcccagttccgcccat
tctccgccccatggctgactaattttttttatttatgcagaggccgaggc
cgcctcggcctctgagctattccagaagtagtgaggaggcttttttggag
gcctagacttttgcagagacggcccaaattcgtaatcatggtcatagctg
tttcctgtgtgaaattgttatccgctcacaattccacacaacatacgagc
cggaagcataaagtgtaaagcctggggtgcctaatgagtgagctaactca
cattaattgcgttgcgctcactgcccgctttccagtcgggaaacctgtcg
tgccagctgcattaatgaatcggccaacgcgcggggagaggcggtttgcg
tattgggcgctcttccgcttcctcgctcactgactcgctgcgctcggtcg
ttcggctgcggcgagcggtatcagctcactcaaaggcggtaatacggtta
tccacagaatcaggggataacgcaggaaagaacatgtgagcaaaaggcca
gcaaaaggccaggaaccgtaaaaaggccgcgttgctggcgtttttccata
ggctccgcccccctgacgagcatcacaaaaatcgacgctcaagtcagagg
tggcgaaacccgacaggactataaagataccaggcgtttccccctggaag
ctccctcgtgcgctctcctgttccgaccctgccgcttaccggatacctgt
ccgcctttctcccttcgggaagcgtggcgctttctcatagctcacgctgt
aggtatctcagttcggtgtaggtcgttcgctccaagctgggctgtgtgca
cgaaccccccgttcagcccgaccgctgcgccttatccggtaactatcgtc
ttgagtccaacccggtaagacacgacttatcgccactggcagcagccact
ggtaacaggattagcagagcgaggtatgtaggcggtgctacagagttctt
gaagtggtggcctaactacggctacactagaaggacagtatttggtatct
gcgctctgctgaagccagttaccttcggaaaaagagttggtagctcttga
tccggcaaacaaaccaccgctggtagcggtggtttttttgtttgcaagca
gcagattacgcgcagaaaaaaaggatctcaagaagatcctttgatctttt
ctacggggtctgacgctcagtggaacgaaaactcacgttaagggattttg
gtcatgagattatcaaaaaggatcttcacctagatccttttaaattaaaa
atgaagttttaaatcaatctaaagtatatatgagtaaacttggtctgaca
gttaccaatgcttaatcagtgaggcacctatctcagcgatctgtctattt
cgttcatccatagttgcctgactccccgtcgtgtagataactacgatacg
ggagggcttaccatctggccccagtgctgcaatgataccgcgagacccac
gctcaccggctccagatttatcagcaataaaccagccagccggaagggcc
gagcgcagaagtggtcctgcaactttatccgcctccatccagtctattaa
ttgttgccgggaagctagagtaagtagttcgccagttaatagtttgcgca
acgttgttgccattgctacaggcatcgtggtgtcacgctcgtcgtttggt
atggcttcattcagctccggttcccaacgatcaaggcgagttacatgatc
ccccatgttgtgcaaaaaagcggttagctccttcggtcctccgatcgttg
tcagaagtaagttggccgcagtgttatcactcatggttatggcagcactg
cataattctcttactgtcatgccatccgtaagatgcttttctgtgactgg
tgagtactcaaccaagtcattctgagaatagtgtatgcggcgaccgagtt
gctcttgcccggcgtcaatacgggataataccgcgccacatagcagaact
ttaaaagtgctcatcattggaaaacgttcttcggggcgaaaactctcaag
gatcttaccgctgttgagatccagttcgatgtaacccactcgtgcaccca
actgatcttcagcatcttttactttcaccagcgtttctgggtgagcaaaa
acaggaaggcaaaatgccgcaaaaaagggaataagggcgacacggaaatg
ttgaatactcatactcttcctttttcaatattattgaagcatttatcagg
gttattgtctcatgagcggatacatatttgaatgtatttagaaaaataaa
caaataggggttccgcgcacatttccccgaaaagtgccacctgacgtcta
agaaaccattattatcatgacattaacctataaaaataggcgtatcacga
ggccctttcgtctcgcgcgtttcggtgatgacggtgaaaacctctgacac
atgcagctcccggagacggtcacagcttgtctgtaagcggatgccgggag
cagacaagcccgtcagggcgcgtcagcgggtgttggcgggtgtcggggct
ggcttaactatgcggcatcagagcagattgtactgagagtgcaccatatg
cggtgtgaaataccgcacagatgcgtaaggagaaaataccgcatcaggcg
ccattcgccattcaggctgcgcaactgttgggaagggcgatcggtgcggg
cctcttcgctattacgccagctggcgaaagggggatgtgctgcaaggcga
ttaagttgggtaacgccagggttttcccagtcacgacgttgtaaaacgac
ggccagtgccaagctg
[SEQ ID No: 5]

Preferably, the genetic construct comprises a nucleic acid sequence substantially as set out in SEQ ID No: 5, or a fragment or variant thereof.

Another embodiment of the genetic construct is shown in FIG. 8H, and is referred to herein as SEQ ID No: 78, as follows:

acgcgtgtagtcttatgcaatactcttgtagtcttgcaacatggtaacga
tgagttagcaacatgccttacaaggagagaaaaagcaccgtgcatgccga
ttggtggaagtaaggtggtacgatcgtgccttattaggaaggcaacagac
gggtctgacatggattggacgaaccactgaattgccgcattgcagagata
ttgtatttaagtgcctagctcgatacaataaacgggtctctctggttaga
ccagatctgagcctgggagctctctggctaactagggaacccactgctta
agcctcaataaagcttgccttgagtgcttcaagtagtgtgtgcccgtctg
ttgtgtgactctggtaactagagatccctcagacccttttagtcagtgtg
gaaaatctctagcagtggcgcccgaacagggacctgaaagcgaaagggaa
accagagctctctcgacgcaggactcggcttgctgaagcgcgcacggcaa
gaggcgaggggcggcgactggtgagtacgccaaaaattttgactagcgga
ggctagaaggagagagatgggtgcgagagcgtcagtattaagcgggggag
aattagatcgcgatgggaaaaaattcggttaaggccagggggaaagaaaa
aatataaattaaaacatatagtatgggcaagcagggagctagaacgattc
gcagttaatcctggcctgttagaaacatcagaaggctgtagacaaatact
gggacagctacaaccatcccttcagacaggatcagaagaacttagatcat
tatataatacagtagcaaccctctattgtgtgcatcaaaggatagagata
aaagacaccaaggaagctttagacaagatagaggaagagcaaaacaaaag
taagaccaccgcacagcaagcggccactgatcttcagacctggaggagga
gatatgagggacaattggagaagtgaattatataaatataaagtagtaaa
aattgaaccattaggagtagcacccaccaaggcaaagagaagagtggtgc
agagagaaaaaagagcagtgggaataggagctttgttccttgggttcttg
ggagcagcaggaagcactatgggcgcagcctcaatgacgctgacggtaca
ggccagacaattattgtctggtatagtgcagcagcagaacaatttgctga
gggctattgaggcgcaacagcatctgttgcaactcacagtctggggcatc
aagcagctccaggcaagaatcctggctgtggaaagatacctaaaggatca
acagctcctggggatttggggttgctctggaaaactcatttgcaccactg
ctgtgccttggaatgctagttggagtaataaatctctggaacagattTgg
aatcacacgacctggatggagtgggacagagaaattaacaattacacaag
cttaatacactccttaattgaagaatcgcaaaaccagcaagaaaagaatg
aacaagaattattggaattagataaatgggcaagtttgtggaattggttt
aacataacaaattggctgtggtatataaaattattcataatgatagtagg
aggcttggtaggtttaagaatagtttttgctgtactttctatagtgaata
gagttaggcagggatattcaccattatcgtttcagacccacctcccaacc
ccgaggggacccgacaggcccgaaggaatagaagaagaaggtggagagag
agacagagacagatccattcgattagtgaacggatctcgacggtATCGGT
taacttttaaaagaaaaggggggattggggggtacagtgcaggggaaaga
atagtagacataatagcaacagacatacaaactaaagaattacaaaaaca
aattacaaaaattcaaaattttatGTCGAGccatagagcccaccgcatcc
ccagcatgcctgctattgtcttcccaatcctcccccttgctgtcctgccc
caccccaccccccagaatagaatgacacctactcagacaatgcgatgcaa
tttcctcattttattaggaaaggacagtgggagtggcaccttccagggtc
aaggaaggcacgggggaggggcaaacaacagatggctggcaactagaagg
cacagtcgCtcgagGGGCCTGTCCGCAAGCACGCTGCATAGCGGGCCTGT
CCGCAAGCACGCTGCACACTGGGCCTGTCCGCAAGCACGCTGCATAGCGG
GCCTGTCCGCAAGCACGCTGCACACTCCCACCCCTAAACAACGTAATGTA
GCCCCACCCCTAAACAACGTAATGCACTCCCACCCCTAAACAACGTAATG
TAGCCCCACCCCTAAACAACGTAATGCACTTGACATCATACCCGTGAAGG
TCTAGCTGACATCATACCCGTGAAGGTCCACTTGACATCATACCCGTGAA
GGTCTAGCTGACATCATACCCGTGAAGGTCCACTACTCCGCCCCCCCGCT
CGTAGCACTCCGCCCCCCCGCTCGCACTACTCCGCCCCCCCGCTCGTAGC
ACTCCGCCCCCCCGCTCGCACTGACGTTACATTCGTGAAGAATGTAGCGA
CGTTACATTCGTGAAGAATGCACTGACGTTACATTCGTGAAGAATGTAGC
GACGTTACATTCGTGAAGAATGCACTGCCCCAGCCGCCGCTGCACTAGCG
CCCCAGCCGCCGCTGCACCACTGCCCCAGCCGCCGCTGCACTAGCGCCCC
AGCCGCCGCTGCACctcgagTCAAACCTTCCTCTTCTTCTTAGGAGGCCT
CAGGCTGCTCTGCCCGCTTTCCAGTCGGGAAACCTGTCGTGCCAGCTGCA
TTAATGAATCGGCCAACGCGCGGGGAGAGGCGGTTTGCGTATTGGGCGCC
AGGGTGGTTTTTCTTTTCACCAGTGAGACGGGCAACAGCTGATTGCCCTT
CACCGCCTGGCCCTGAGAGAGTTGCAGCAAGCGGTCCACGCTGGTTTGCC
CCAGCAGGCGAAAATCCTGTTTGATGGTGGTTAACGGCGGGATATAACAT
GAGCTGTCTTCGGTATCGTCGTATCCCACTACCGAGATATCCGCACCAAC
GCGCAGCCCGGACTCGGTAATGGCGCGCATTGCGCCCAGCGCCATCTGAT
CGTTGGCAACCAGCATCGCAGTGGGAACGATGCCCTCATTCAGCATTTGC
ATGGTTTGTTGAAAACCGGACATGGCACTCCAGTCGCCTTCCCGTTCCGC
TATCGGCTGAATTTGATTGCGAGTGAGATATTTATGCCAGCCAGCCAGAC
GCAGACGCGCCGAGACAGAACTTAATGGGCCCGCTAACAGCGCGATTTGC
TGGTGACCCAATGCGACCAGATGCTCCACGCCCAGTCGCGTACCGTCTTC
ATGGGAGAAAATAATACTGTTGATGGGTGTCTGGTCAGAGACATCAAGAA
ATAACGCCGGAACATTAGTGCAGGCAGCTTCCACAGCAATGGCATCCTGG
TCATCCAGCGGATAGTTAATGATCAGCCCACTGACGCGTTGCGCGAGAAG
ATTGTGCACCGCCGCTTTACAGGCTTCGACGCCGCTTCGTTCTACCATCG
ACACCACCACGCTGGCACCCAGTTGATCGGCGCGAGATTTAATCGCCGCG
ACAATTTGCGACGGCGCGTGCAGGGCCAGACTGGAGGTGGCAACGCCAAT
CAGCAACGACTGTTTGCCCGCCAGTTGTTGTGCCACGCGGTTGGGAATGT
AATTCAGCTCCGCCATCGCCGCTTCCACTTTTTCCCGCGTTTTCGCAGAA
ACGTGGCTGGCCTGGTTCACCACGCGGGAAACGGTCTGATAAGAGACACC
GGCATACTCTGCGACATCGTATAACGTTACTGGTTTCATGGTGGGAGGGT
ACcTAGGTAAGCTTGGGCTGCAGGTCGAAAGGCCCGGAGATGAGGAAGAG
GAGAACAGCGCGGCAGACGTGCGCTTTTGAAGCGTGCAGAATGCCGGGCC
TCCGGAGGACCTTCGGGCGCCCGCCCCGCCCCTGAGCCCGCCCCTGAGCC
CGCCCCCGGACCCACCCCTTCCCAGCCTCTGAGCCCAGAAAGCGAAGGAG
CAAAGCTGCTATTGGCCGCTGCCCCAAAGGCCTACCCGCTTCCATTGCTC
AGCGGTGCTGTCCATCTGCACGAGACTAGTGAGACGTGCTACTTCCATTT
GTCACGTCCTGCACGACGCGAGCTGCGGGGCGGGGGGGAACTTCCTGACT
AGGGGAGGAGTAGAAGGTGGCGCGAAGGGGCCACCAAAGAACGGAGCCGG
TTGGCGCCTACCGGTGGATGTGGAATGTGTGCGAGGCCAGAGGCCACTTG
TGTAGCGCCAAGTGCCCAGCGGGGCTGCTAAAGCGCATGCTCCAGACTGC
CTTGGGAAAAGCGCCTCCCCTACCCGGTAGAAGCTAGCGGCTCCGGTGCC
CGTCAGTGGGCAGAGCGCACATCGCCCACAGTCCCCGAGAAGTTGTGGGG
AGGGGTCGGCAATTGAACCGGTGCCTAGAGAAGGTGGCGCGGGGTAAACT
GGGAAAGTGATGTCGTGTACTGGCTCCGCCTTTTTCCCGAGGGTGGGGGA
GAACCGTATATAAGTGCAGTAGTCGCCGTGAACGTTCTTTTTCGCAACGG
GTTTGCCGCCAGAACACAGGTAAGTGCCGTGTGTGGTTCCCGCGGGCCTG
GCCTCTTTACGGGTTATGGCCCTTGCGTGCCTTGAATTACTTCCACCTGG
CTGCAGTACGTGATTCTTGATCCCGAGCTTCGGGTTGGAAGTGGGTGGGA
GAGTTCGAGGCCTTGCGCTTAAGGAGCCCCTTCGCCTCGTGCTTGAGTTG
AGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGG
GCCAAGATCTGCACACTGGTATTTCGGTTTTTGGGGCCGCGGGCGGCGAC
GGGGCCCGTGCGTCCCAGCGCACATGTTCGGCGAGGCGGGGCCTGCGAGC
GCGGCCACCGAGAATCGGACGGGGGTAGTCTCAAGCTGGCCGGCCTGCTC
TGGTGCCTGGCCTCGCGCCGCCGTGTATCGCCCCGCCCTGGGCGGCAAGG
CTGGCCCGGTCGGCACCAGTTGCGTGAGCGGAAAGATGGCCGCTTCCCGG
CCCTGCTGCAGGGAGCTCAAAATGGAGGACGCGGCGCTCGGGAGAGCGGG
CGGGTGAGTCACCCACACAAAGGAAAAGGGCCTTTCCGTCCTCAGCCGTC
GCTTCATGTGACTCCACGGAGTACCGGGCGCCGTCCAGGCACCTCGATTA
GTTCTCGACCTTTTGGAGTACGTCGTCTTTAGGTTGGGGGGAGGGGTTTT
ATGCGATGGAGTTTCCCCACACTGAGTGGGTGGAGACTGAAGTTAGGCCA
GCTTGGCACTTGATGTAATTCTCCTTGGAATTTGCCCTTTTTGAGTTTGG
ATCTTGGTTCATTCTCAAGCCTCAGACAGTGGTTCAAAGTTTTTTTCTTC
CATTTCAGGTGTCGTGAtctagagctagcgAATTCGTAAATATAAAATTT
ACTAGGTTGTGGAATTGTGAGCGCTCACAATTCCACAGTCGACCCTAGGT
TGTGGAATTGTGAGCGCTCACAATTCCACAGTCGACCCTAGGTTGTGGAA
TTGTGAGCGCTCACAATTCCACAGTCGACCCTAGTGTATAATGTGTTAAA
CTACGGATCCGTCTCCCATTAGGCCTACAATGGTGAGACAAGTAGCCAAC
AGGGAAGGGTTGCAAATATCATTTGGGCACACCTATGATAATATTGATGA
AGCAGACAGTATTCAGCAAGTAACTGAGAGGTGGGAAGCTCAAAGCCAAA
GTCCTAATGTGCAGTCAGGTGAATTTATTGAAAAATTTGAGGCTCCTGGT
GGTGCAAATCAAAGAACTGCTCCTCAGGGATCCTAATTGTTTGTGTATTT
TAGGCggccgctgagggcagaggaagtcttctaacatgcggtgacgtgga
ggagaatcccggcccttccggaATGGAGAGCGACGAGAGCGGCCTGCCCG
CCATGGAGATCGAGTGCCGCATCACCGGCACCCTGAACGGCGTGGAGTTC
GAGCTGGTGGGCGGCGGAGAGGGCACCCCCAAGCAGGGCCGCATGACCAA
CAAGATGAAGAGCACCAAAGGCGCCCTGACCTTCAGCCCCTACCTGCTGA
GCCACGTGATGGGCTACGGCTTCTACCACTTCGGCACCTACCCCAGCGGC
TACGAGAACCCCTTCCTGCACGCCATCAACAACGGCGGCTACACCAACAC
CCGCATCGAGAAGTACGAGGACGGCGGCGTGCTGCACGTGAGCTTCAGCT
ACCGCTACGAGGCCGGCCGCGTGATCGGCGACTTCAAGGTGGTGGGCACC
GGCTTCCCCGAGGACAGCGTGATCTTCACCGACAAGATCATCCGCAGCAA
CGCCACCGTGGAGCACCTGCACCCCATGGGCGATAACGTGCTGGTGGGCA
GCTTCGCCCGCACCTTCAGCCTGCGCGACGGCGGCTACTACAGCTTCGTG
GTGGACAGCCACATGCACTTCAAGAGCGCCATCCACCCCAGCATCCTGCA
GAACGGGGGCCCCATGTTCGCCTTCCGCCGCGTGGAGGAGCTGCACAGCA
ACACCGAGCTGGGCATCGTGGAGTACCAGCACGCCTTCAAGACCCCCATC
GCCTTCGCCAGATCCCGCGCTCAGTCGTCCAATTCTGCCGTGGACGGCAC
CGCCGGACCCGGCTCCACCGGATCTCGCTAGAGCTGAATCtaagtcgaca
atcaacctctggattacaaaatttgtgaaagattgactggtattcttaac
tatgttgctccttttacgctatgtggatacgctgctttaatgcctttgta
tcatgctattgcttcccgtatggctttcattttctcctccttgtataaat
cctggttgctgtctctttatgaggagttgtggcccgttgtcaggcaacgt
ggcgtggtgtgcactgtgtttgctgacgcaacccccactggttggggcat
tgccaccacctgtcagctcctttccgggactttcgctttccccctcccta
ttgccacggcggaactcatcgccgcctgccttgcccgctgctggacaggg
gctcggctgttgggcactgacaattccgtggtgttgtcggggaaatcatc
gtcctttccttggctgctcgcctgtgttgccacctggattctgcgcggga
cgtccttctgctacgtcccttcggccctcaatccagcggaccttccttcc
cgcggcctgctgccggctctgcggcctcttccgcgtcttcgccttcgccc
tcagacgagtcggatctccctttgggccgcctccccgcccgggACACACC
AAGGATAATTTCTCCCGATACACACCAAGGATAATTTCTCCTCACACACA
CCAAGGATAATTTCTCCCGATACACACCAAGGATAATTTCTCCTCACAAC
TGGATGTCCCTGTATGATTCGATAACTGGATGTCCCTGTATGATTTCACA
ACTGGATGTCCCTGTATGATTCGATAACTGGATGTCCCTGTATGATTTCA
CAAAGAGGTTAACCATGTATTATCGATAAAGAGGTTAACCATGTATTATT
CACAAAGAGGTTAACCATGTATTATCGATAAAGAGGTTAACCATGTATTA
TTCACGGTTAGTGGACCGTGTTACATACGATGGTTAGTGGACCGTGTTAC
ATATCACGGTTAGTGGACCGTGTTACATACGATGGTTAGTGGACCGTGTT
ACATATCACGAATTCACCAAGGGCAACCTCTCGATGAATTCACCAAGGGC
AACCTCTTCACGAATTCACCAAGGGCAACCTCTCGATGAATTCACCAAGG
GCAACCTCTTCACAACTCCTGTATGAAGCCGTTCCGATAACTCCTGTATG
AAGCCGTTCTCACAACTCCTGTATGAAGCCGTTCCGATAACTCCTGTATG
AAGCCGTTCTCACATGGCAATATGTTGGCATAGCACGATATGGCAATATG
TTGGCATAGCATCACATGGCAATATGTTGGCATAGCACGATATGGCAATA
TGTTGGCATAGCATCACGCACAGCAAGTGTAGACAGGCACGATGCACAGC
AAGTGTAGACAGGCATCACGCACAGCAAGTGTAGACAGGCACGATGCACA
GCAAGTGTAGACAGGCATCACAACATACAAAGGGTATCCTCTCGATAACA
TACAAAGGGTATCCTCTTCACAACATACAAAGGGTATCCTCTCGATAACA
TACAAAGGGTATCCTCTTCACCTCCCTTCTTTCCTCCCGTCTTCGATCTC
CCTTCTTTCCTCCCGTCTTTCACCTCCCTTCTTTCCTCCCGTCTTCGATC
TCCCTTCTTTCCTCCCGTCTTcccgggtacctttaagaccaatgacttac
aaggcagctgtagatcttagccactttttaaaagaaaaggggggactgga
agggctaattcactcccaacgaaaataagatctgctttttgcttgtactg
ggtctctctggttagaccagatctgagcctgggagctctctggctaacta
gggaacccactgcttaagcctcaataaagcttgccttgagtgcttcaagt
agtgtgtgcccgtctgttgtgtgactctggtaactagagatccctcagac
ccttttagtcagtgtggaaaatctctagcagtagtagttcatgtcatctt
attattcagtatttataacttgcaaagaaatgaatatcagagagtgagag
gaacttgtttattgcagcttataatggttacaaataaagcaatagcatca
caaatttcacaaataaagcatttttttcactgcattctagttgtggtttg
tccaaactcatcaatgtatcttatcatgtctggctctagctatcccgccc
ctaactccgcccagttccgcccattctccgccccatggctgactaatttt
ttttatttatgcagaggccgaggccgcctcggcctctgagctattccaga
agtagtgaggaggcttttttggaggcctagacttttgcagagacggccca
aattcgtaatcatggtcatagctgtttcctgtgtgaaattgttatccgct
cacaattccacacaacatacgagccggaagcataaagtgtaaagcctggg
gtgcctaatgagtgagctaactcacattaattgcgttgcgctcactgccc
gctttccagtcgggaaacctgtcgtgccagctgcattaatgaatcggcca
acgcgcggggagaggcggtttgcgtattgggcgctcttccgcttcctcgc
tcactgactcgctgcgctcggtcgttcggctgcggcgagcggtatcagct
cactcaaaggcggtaatacggttatccacagaatcaggggataacgcagg
aaagaacatgtgagcaaaaggccagcaaaaggccaggaaccgtaaaaagg
ccgcgttgctggcgtttttccataggctccgcccccctgacgagcatcac
aaaaatcgacgctcaagtcagaggtggcgaaacccgacaggactataaag
ataccaggcgtttccccctggaagctccctcgtgcgctctcctgttccga
ccctgccgcttaccggatacctgtccgcctttctcccttcgggaagcgtg
gcgctttctcatagctcacgctgtaggtatctcagttcggtgtaggtcgt
tcgctccaagctgggctgtgtgcacgaaccccccgttcagcccgaccgct
gcgccttatccggtaactatcgtcttgagtccaacccggtaagacacgac
ttatcgccactggcagcagccactggtaacaggattagcagagcgaggta
tgtaggcggtgctacagagttcttgaagtggtggcctaactacggctaca
ctagaaggacagtatttggtatctgcgctctgctgaagccagttaccttc
ggaaaaagagttggtagctcttgatccggcaaacaaaccaccgctggtag
cggtggtttttttgtttgcaagcagcagattacgcgcagaaaaaaaggat
ctcaagaagatcctttgatcttttctacggggtctgacgctcagtggaac
gaaaactcacgttaagggattttggtcatgagattatcaaaaaggatctt
cacctagatccttttaaattaaaaatgaagttttaaatcaatctaaagta
tatatgagtaaacttggtctgacagttaccaatgcttaatcagtgaggca
cctatctcagcgatctgtctatttcgttcatccatagttgcctgactccc
cgtcgtgtagataactacgatacgggagggcttaccatctggccccagtg
ctgcaatgataccgcgagacccacgctcaccggctccagatttatcagca
ataaaccagccagccggaagggccgagcgcagaagtggtcctgcaacttt
atccgcctccatccagtctattaattgttgccgggaagctagagtaagta
gttcgccagttaatagtttgcgcaacgttgttgccattgctacaggcatc
gtggtgtcacgctcgtcgtttggtatggcttcattcagctccggttccca
acgatcaaggcgagttacatgatcccccatgttgtgcaaaaaagcggtta
gctccttcggtcctccgatcgttgtcagaagtaagttggccgcagtgtta
tcactcatggttatggcagcactgcataattctcttactgtcatgccatc
cgtaagatgcttttctgtgactggtgagtactcaaccaagtcattctgag
aatagtgtatgcggcgaccgagttgctcttgcccggcgtcaatacgggat
aataccgcgccacatagcagaactttaaaagtgctcatcattggaaaacg
ttcttcggggcgaaaactctcaaggatcttaccgctgttgagatccagtt
cgatgtaacccactcgtgcacccaactgatcttcagcatcttttactttc
accagcgtttctgggtgagcaaaaacaggaaggcaaaatgccgcaaaaaa
gggaataagggcgacacggaaatgttgaatactcatactcttcctttttc
aatattattgaagcatttatcagggttattgtctcatgagcggatacata
tttgaatgtatttagaaaaataaacaaataggggttccgcgcacatttcc
ccgaaaagtgccacctgacgtctaagaaaccattattatcatgacattaa
cctataaaaataggcgtatcacgaggccctttcgtctcgcgcgtttcggt
gatgacggtgaaaacctctgacacatgcagctcccggagacggtcacagc
ttgtctgtaagcggatgccgggagcagacaagcccgtcagggcgcgtcag
cgggtgttggcgggtgtcggggctggcttaactatgcggcatcagagcag
attgtactgagagtgcaccatatgcggtgtgaaataccgcacagatgcgt
aaggagaaaataccgcatcaggcgccattcgccattcaggctgcgcaact
gttgggaagggcgatcggtgcgggcctcttcgctattacgccagctggcg
aaagggggatgtgctgcaaggcgattaagttgggtaacgccagggttttc
ccagtcacgacgttgtaaaacgacggccagtgccaagctg
[SEQ ID No: 78]

Preferably, the genetic construct comprises a nucleic acid sequence substantially as set out in SEQ ID No: 78, or a fragment or variant thereof.

Another embodiment of the genetic construct is shown in FIG. 8D and may comprise a Bax encoding sequence and be represented by SEQ ID No: 22, as follows:

acgcgtgtagtcttatgcaatactcttgtagtcttgcaacatggtaacga
tgagttagcaacatgccttacaaggagagaaaaagcaccgtgcatgccga
ttggtggaagtaaggtggtacgatcgtgccttattaggaaggcaacagac
gggtctgacatggattggacgaaccactgaattgccgcattgcagagata
ttgtatttaagtgcctagctcgatacaataaacgggtctctctggttaga
ccagatctgagcctgggagctctctggctaactagggaacccactgctta
agcctcaataaagcttgccttgagtgcttcaagtagtgtgtgcccgtctg
ttgtgtgactctggtaactagagatccctcagacccttttagtcagtgtg
gaaaatctctagcagtggcgcccgaacagggacctgaaagcgaaagggaa
accagagctctctcgacgcaggactcggcttgctgaagcgcgcacggcaa
gaggcgaggggcggcgactggtgagtacgccaaaaattttgactagcgga
ggctagaaggagagagatgggtgcgagagcgtcagtattaagcgggggag
aattagatcgcgatgggaaaaaattcggttaaggccagggggaaagaaaa
aatataaattaaaacatatagtatgggcaagcagggagctagaacgattc
gcagttaatcctggcctgttagaaacatcagaaggctgtagacaaatact
gggacagctacaaccatcccttcagacaggatcagaagaacttagatcat
tatataatacagtagcaaccctctattgtgtgcatcaaaggatagagata
aaagacaccaaggaagctttagacaagatagaggaagagcaaaacaaaag
taagaccaccgcacagcaagcggccactgatcttcagacctggaggagga
gatatgagggacaattggagaagtgaattatataaatataaagtagtaaa
aattgaaccattaggagtagcacccaccaaggcaaagagaagagtggtgc
agagagaaaaaagagcagtgggaataggagctttgttccttgggttcttg
ggagcagcaggaagcactatgggcgcagcctcaatgacgctgacggtaca
ggccagacaattattgtctggtatagtgcagcagcagaacaatttgctga
gggctattgaggcgcaacagcatctgttgcaactcacagtctggggcatc
aagcagctccaggcaagaatcctggctgtggaaagatacctaaaggatca
acagctcctggggatttggggttgctctggaaaactcatttgcaccactg
ctgtgccttggaatgctagttggagtaataaatctctggaacagattTgg
aatcacacgacctggatggagtgggacagagaaattaacaattacacaag
cttaatacactccttaattgaagaatcgcaaaaccagcaagaaaagaatg
aacaagaattattggaattagataaatgggcaagtttgtggaattggttt
aacataacaaattggctgtggtatataaaattattcataatgatagtagg
aggcttggtaggtttaagaatagtttttgctgtactttctatagtgaata
gagttaggcagggatattcaccattatcgtttcagacccacctcccaacc
ccgaggggacccgacaggcccgaaggaatagaagaagaaggtggagagag
agacagagacagatccattcgattagtgaacggatctcgacggtATCGGT
taacttttaaaagaaaaggggggattggggggtacagtgcaggggaaaga
atagtagacataatagcaacagacatacaaactaaagaattacaaaaaca
aattacaaaaattcaaaattttatGTCGAGccatagagcccaccgcatcc
ccagcatgcctgctattgtcttcccaatcctcccccttgctgtcctgccc
caccccaccccccagaatagaatgacacctactcagacaatgcgatgcaa
tttcctcattttattaggaaaggacagtgggagtggcaccttccagggtc
aaggaaggcacgggggaggggcaaacaacagatggctggcaactagaagg
cacagtcgCtcgagAGTAAAACACTACAACGTCGATAGCAGTAAAACACT
ACAACGTCGACACTAGTAAAACACTACAACGTCGATAGCAGTAAAACACT
ACAACGTCGACACTAGTGTCACTTGGCCAGAGAAATAGCAGTGTCACTTG
GCCAGAGAAACACTAGTGTCACTTGGCCAGAGAAATAGCAGTGTCACTTG
GCCAGAGAAActcgagTCAAACCTTCCTCTTCTTCTTAGGAGGCCTCAGG
CTGCTCTGCCCGCTTTCCAGTCGGGAAACCTGTCGTGCCAGCTGCATTAA
TGAATCGGCCAACGCGCGGGGAGAGGCGGTTTGCGTATTGGGCGCCAGGG
TGGTTTTTCTTTTCACCAGTGAGACGGGCAACAGCTGATTGCCCTTCACC
GCCTGGCCCTGAGAGAGTTGCAGCAAGCGGTCCACGCTGGTTTGCCCCAG
CAGGCGAAAATCCTGTTTGATGGTGGTTAACGGCGGGATATAACATGAGC
TGTCTTCGGTATCGTCGTATCCCACTACCGAGATATCCGCACCAACGCGC
AGCCCGGACTCGGTAATGGCGCGCATTGCGCCCAGCGCCATCTGATCGTT
GGCAACCAGCATCGCAGTGGGAACGATGCCCTCATTCAGCATTTGCATGG
TTTGTTGAAAACCGGACATGGCACTCCAGTCGCCTTCCCGTTCCGCTATC
GGCTGAATTTGATTGCGAGTGAGATATTTATGCCAGCCAGCCAGACGCAG
ACGCGCCGAGACAGAACTTAATGGGCCCGCTAACAGCGCGATTTGCTGGT
GACCCAATGCGACCAGATGCTCCACGCCCAGTCGCGTACCGTCTTCATGG
GAGAAAATAATACTGTTGATGGGTGTCTGGTCAGAGACATCAAGAAATAA
CGCCGGAACATTAGTGCAGGCAGCTTCCACAGCAATGGCATCCTGGTCAT
CCAGCGGATAGTTAATGATCAGCCCACTGACGCGTTGCGCGAGAAGATTG
TGCACCGCCGCTTTACAGGCTTCGACGCCGCTTCGTTCTACCATCGACAC
CACCACGCTGGCACCCAGTTGATCGGCGCGAGATTTAATCGCCGCGACAA
TTTGCGACGGCGCGTGCAGGGCCAGACTGGAGGTGGCAACGCCAATCAGC
AACGACTGTTTGCCCGCCAGTTGTTGTGCCACGCGGTTGGGAATGTAATT
CAGCTCCGCCATCGCCGCTTCCACTTTTTCCCGCGTTTTCGCAGAAACGT
GGCTGGCCTGGTTCACCACGCGGGAAACGGTCTGATAAGAGACACCGGCA
TACTCTGCGACATCGTATAACGTTACTGGTTTCATGGTGGGAGGGTACcT
AGGTAAGCTTGGGCTGCAGGTCGAAAGGCCCGGAGATGAGGAAGAGGAGA
ACAGCGCGGCAGACGTGCGCTTTTGAAGCGTGCAGAATGCCGGGCCTCCG
GAGGACCTTCGGGCGCCCGCCCCGCCCCTGAGCCCGCCCCTGAGCCCGCC
CCCGGACCCACCCCTTCCCAGCCTCTGAGCCCAGAAAGCGAAGGAGCAAA
GCTGCTATTGGCCGCTGCCCCAAAGGCCTACCCGCTTCCATTGCTCAGCG
GTGCTGTCCATCTGCACGAGACTAGTGAGACGTGCTACTTCCATTTGTCA
CGTCCTGCACGACGCGAGCTGCGGGGCGGGGGGGAACTTCCTGACTAGGG
GAGGAGTAGAAGGTGGCGCGAAGGGGCCACCAAAGAACGGAGCCGGTTGG
CGCCTACCGGTGGATGTGGAATGTGTGCGAGGCCAGAGGCCACTTGTGTA
GCGCCAAGTGCCCAGCGGGGCTGCTAAAGCGCATGCTCCAGACTGCCTTG
GGAAAAGCGCCTCCCCTACCCGGTAGAAGCTAGCGGCTCCGGTGCCCGTC
AGTGGGCAGAGCGCACATCGCCCACAGTCCCCGAGAAGTTGTGGGGAGGG
GTCGGCAATTGAACCGGTGCCTAGAGAAGGTGGCGCGGGGTAAACTGGGA
AAGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTTT
GCCGCCAGAACACAGGTAAGTGCCGTGTGTGGTTCCCGCGGGCCTGGCCT
CTTTACGGGTTATGGCCCTTGCGTGCCTTGAATTACTTCCACCTGGCTGC
AGTACGTGATTCTTGATCCCGAGCTTCGGGTTGGAAGTGGGTGGGAGAGT
TCGAGGCCTTGCGCTTAAGGAGCCCCTTCGCCTCGTGCTTGAGTTGAGGC
CTGGCCTGGGCGCTGGGGCCGCCGCGTGCGAATCTGGTGGCACCTTCGCG
CCTGTCTCGCTGCTTTCGATAAGTCTCTAGCCATTTAAAATTTTTGATGA
CCTGCTGCGACGCTTTTTTTCTGGCAAGATAGTCTTGTAAATGCGGGCCA
AGATCTGCACACTGGTATTTCGGTTTTTGGGGCCGCGGGCGGCGACGGGG
CCCGTGCGTCCCAGCGCACATGTTCGGCGAGGCGGGGCCTGCGAGCGCGG
CCACCGAGAATCGGACGGGGGTAGTCTCAAGCTGGCCGGCCTGCTCTGGT
GCCTGGCCTCGCGCCGCCGTGTATCGCCCCGCCCTGGGCGGCAAGGCTGG
CCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGG
TGAGTCACCCACACAAAGGAAAAGGGCCTTTCCGTCCTCAGCCGTCGCTT
CATGTGACTCCACGGAGTACCGGGCGCCGTCCAGGCACCTCGATTAGTTC
TCGACCTTTTGGAGTACGTCGTCTTTAGGTTGGGGGGAGGGGTTTTATGC
GATGGAGTTTCCCCACACTGAGTGGGTGGAGACTGAAGTTAGGCCAGCTT
GGCACTTGATGTAATTCTCCTTGGAATTTGCCCTTTTTGAGTTTGGATCT
TGGTTCATTCTCAAGCCTCAGACAGTGGTTCAAAGTTTTTTTCTTCCATT
TCAGGTGTCGTGAtctagagctagcgAATTCGTAAATATAAAATTTACTA
GGTTGTGGAATTGTGAGCGCTCACAATTCCACAGTCGACCCTAGGTTGTG
GAATTGTGAGCGCTCACAATTCCACAGTCGACCCTAGGTTGTGGAATTGT
GAGCGCTCACAATTCCACAGTCGACCCTAGTGTATAATGTGTTAAACTAC
GGATCCGTCTCCCATTAGGCCTACAATGGTGAGACAAGTAGCCAACAGGG
AAGGGTTGCAAATATCATTTGGGCACACCTATGATAATATTGATGAAGCA
GACAGTATTCAGCAAGTAACTGAGAGGTGGGAAGCTCAAAGCCAAAGTCC
TAATGTGCAGTCAGGTGAATTTATTGAAAAATTTGAGGCTCCTGGTGGTG
CAAATCAAAGAACTGCTCCTCAGGGATCCTAATTGTTTGTGTATTTTAGG
CggccgcCGCGATCGCCATGGACGGGTCCGGGGAGCAGCCCAGAGGCGGG
GGGCCCACCAGCTCTGAGCAGATCATGAAGACAGGGGCCCTTTTGCTTCA
GGGTTTCATCCAGGATCGAGCAGGGCGAATGGGGGGGGAGGCACCCGAGC
TGGCCCTGGACCCGGTGCCTCAGGATGCGTCCACCAAGAAGCTGAGCGAG
TGTCTCAAGCGCATCGGGGACGAACTGGACAGTAACATGGAGCTGCAGAG
GATGATTGCCGCCGTGGACACAGACTCCCCCCGAGAGGTCTTTTTCCGAG
TGGCAGCTGACATGTTTTCTGACGGCAACTTCAACTGGGGCCGGGTTGTC
GCCCTTTTCTACTTTGCCAGCAAACTGGTGCTCAAGGCCCTGTGCACCAA
GGTGCCGGAACTGATCAGAACCATCATGGGCTGGACATTGGACTTCCTCC
GGGAGCGGCTGTTGGGCTGGATCCAAGACCAGGGTGGTTGGGGGCTGCCC
CTGGCCGAGTCACTGAAGCGACTGATGTCCCTGTCTCCAGGACGGCCTCC
TCTCCTACTTTGGGACGCCCACGTGGCAGACCGTGACCATCTTTGTGGCG
GGAGTGCTCACCGCCTCACTCACCATCTGGAAGAAGATGGGCTGAGGCCC
CCAGCTGCCTTGGACTGTGTTGCGGCCGCtgagggcagaggaagtcttct
aacatgcggtgacgtggaggagaatcccggcccttccggaATGGAGAGCG
ACGAGAGCGGCCTGCCCGCCATGGAGATCGAGTGCCGCATCACCGGCACC
CTGAACGGCGTGGAGTTCGAGCTGGTGGGCGGCGGAGAGGGCACCCCCAA
GCAGGGCCGCATGACCAACAAGATGAAGAGCACCAAAGGCGCCCTGACCT
TCAGCCCCTACCTGCTGAGCCACGTGATGGGCTACGGCTTCTACCACTTC
GGCACCTACCCCAGCGGCTACGAGAACCCCTTCCTGCACGCCATCAACAA
CGGCGGCTACACCAACACCCGCATCGAGAAGTACGAGGACGGCGGCGTGC
TGCACGTGAGCTTCAGCTACCGCTACGAGGCCGGCCGCGTGATCGGCGAC
TTCAAGGTGGTGGGCACCGGCTTCCCCGAGGACAGCGTGATCTTCACCGA
CAAGATCATCCGCAGCAACGCCACCGTGGAGCACCTGCACCCCATGGGCG
ATAACGTGCTGGTGGGCAGCTTCGCCCGCACCTTCAGCCTGCGCGACGGC
GGCTACTACAGCTTCGTGGTGGACAGCCACATGCACTTCAAGAGCGCCAT
CCACCCCAGCATCCTGCAGAACGGGGGCCCCATGTTCGCCTTCCGCCGCG
TGGAGGAGCTGCACAGCAACACCGAGCTGGGCATCGTGGAGTACCAGCAC
GCCTTCAAGACCCCCATCGCCTTCGCCAGATCCCGCGCTCAGTCGTCCAA
TTCTGCCGTGGACGGCACCGCCGGACCCGGCTCCACCGGATCTCGCTAGA
GCTGAATCtaagtcgacaatcaacctctggattacaaaatttgtgaaaga
ttgactggtattcttaactatgttgctccttttacgctatgtggatacgc
tgctttaatgcctttgtatcatgctattgcttcccgtatggctttcattt
tctcctccttgtataaatcctggttgctgtctctttatgaggagttgtgg
cccgttgtcaggcaacgtggcgtggtgtgcactgtgtttgctgacgcaac
ccccactggttggggcattgccaccacctgtcagctcctttccgggactt
tcgctttccccctccctattgccacggcggaactcatcgccgcctgcctt
gcccgctgctggacaggggctcggctgttgggcactgacaattccgtggt
gttgtcggggaaatcatcgtcctttccttggctgctcgcctgtgttgcca
cctggattctgcgcgggacgtccttctgctacgtcccttcggccctcaat
ccagcggaccttccttcccgcggcctgctgccggctctgcggcctcttcc
gcgtcttcgccttcgccctcagacgagtcggatctccctttgggccgcct
ccccgcccgggTAACCGATTTCAGATGGTGCTACGATTAACCGATTTCAG
ATGGTGCTATCACTAACCGATTTCAGATGGTGCTACGATTAACCGATTTC
AGATGGTGCTATCACGGGAGTGAAGACACGGAGCCAGACGATGGGAGTGA
AGACACGGAGCCAGATCACGGGAGTGAAGACACGGAGCCAGACGATGGGA
GTGAAGACACGGAGCCAGAcccgggtacctttaagaccaatgacttacaa
ggcagctgtagatcttagccactttttaaaagaaaaggggggactggaag
ggctaattcactcccaacgaaaataagatctgctttttgcttgtactggg
tctctctggttagaccagatctgagcctgggagctctctggctaactagg
gaacccactgcttaagcctcaataaagcttgccttgagtgcttcaagtag
tgtgtgcccgtctgttgtgtgactctggtaactagagatccctcagaccc
ttttagtcagtgtggaaaatctctagcagtagtagttcatgtcatcttat
tattcagtatttataacttgcaaagaaatgaatatcagagagtgagagga
acttgtttattgcagcttataatggttacaaataaagcaatagcatcaca
aatttcacaaataaagcatttttttcactgcattctagttgtggtttgtc
caaactcatcaatgtatcttatcatgtctggctctagctatcccgcccct
aactccgcccagttccgcccattctccgccccatggctgactaatttttt
ttatttatgcagaggccgaggccgcctcggcctctgagctattccagaag
tagtgaggaggcttttttggaggcctagacttttgcagagacggcccaaa
ttcgtaatcatggtcatagctgtttcctgtgtgaaattgttatccgctca
caattccacacaacatacgagccggaagcataaagtgtaaagcctggggt
gcctaatgagtgagctaactcacattaattgcgttgcgctcactgcccgc
tttccagtcgggaaacctgtcgtgccagctgcattaatgaatcggccaac
gcgcggggagaggcggtttgcgtattgggcgctcttccgcttcctcgctc
actgactcgctgcgctcggtcgttcggctgcggcgagcggtatcagctca
ctcaaaggcggtaatacggttatccacagaatcaggggataacgcaggaa
agaacatgtgagcaaaaggccagcaaaaggccaggaaccgtaaaaaggcc
gcgttgctggcgtttttccataggctccgcccccctgacgagcatcacaa
aaatcgacgctcaagtcagaggtggcgaaacccgacaggactataaagat
accaggcgtttccccctggaagctccctcgtgcgctctcctgttccgacc
ctgccgcttaccggatacctgtccgcctttctcccttcgggaagcgtggc
gctttctcatagctcacgctgtaggtatctcagttcggtgtaggtcgttc
gctccaagctgggctgtgtgcacgaaccccccgttcagcccgaccgctgc
gccttatccggtaactatcgtcttgagtccaacccggtaagacacgactt
atcgccactggcagcagccactggtaacaggattagcagagcgaggtatg
taggcggtgctacagagttcttgaagtggtggcctaactacggctacact
agaaggacagtatttggtatctgcgctctgctgaagccagttaccttcgg
aaaaagagttggtagctcttgatccggcaaacaaaccaccgctggtagcg
gtggtttttttgtttgcaagcagcagattacgcgcagaaaaaaaggatct
caagaagatcctttgatcttttctacggggtctgacgctcagtggaacga
aaactcacgttaagggattttggtcatgagattatcaaaaaggatcttca
cctagatccttttaaattaaaaatgaagttttaaatcaatctaaagtata
tatgagtaaacttggtctgacagttaccaatgcttaatcagtgaggcacc
tatctcagcgatctgtctatttcgttcatccatagttgcctgactccccg
tcgtgtagataactacgatacgggagggcttaccatctggccccagtgct
gcaatgataccgcgagacccacgctcaccggctccagatttatcagcaat
aaaccagccagccggaagggccgagcgcagaagtggtcctgcaactttat
ccgcctccatccagtctattaattgttgccgggaagctagagtaagtagt
tcgccagttaatagtttgcgcaacgttgttgccattgctacaggcatcgt
ggtgtcacgctcgtcgtttggtatggcttcattcagctccggttcccaac
gatcaaggcgagttacatgatcccccatgttgtgcaaaaaagcggttagc
tccttcggtcctccgatcgttgtcagaagtaagttggccgcagtgttatc
actcatggttatggcagcactgcataattctcttactgtcatgccatccg
taagatgcttttctgtgactggtgagtactcaaccaagtcattctgagaa
tagtgtatgcggcgaccgagttgctcttgcccggcgtcaatacgggataa
taccgcgccacatagcagaactttaaaagtgctcatcattggaaaacgtt
cttcggggcgaaaactctcaaggatcttaccgctgttgagatccagttcg
atgtaacccactcgtgcacccaactgatcttcagcatcttttactttcac
cagcgtttctgggtgagcaaaaacaggaaggcaaaatgccgcaaaaaagg
gaataagggcgacacggaaatgttgaatactcatactcttcctttttcaa
tattattgaagcatttatcagggttattgtctcatgagcggatacatatt
tgaatgtatttagaaaaataaacaaataggggttccgcgcacatttcccc
gaaaagtgccacctgacgtctaagaaaccattattatcatgacattaacc
tataaaaataggcgtatcacgaggccctttcgtctcgcgcgtttcggtga
tgacggtgaaaacctctgacacatgcagctcccggagacggtcacagctt
gtctgtaagcggatgccgggagcagacaagcccgtcagggcgcgtcagcg
ggtgttggcgggtgtcggggctggcttaactatgcggcatcagagcagat
tgtactgagagtgcaccatatgcggtgtgaaataccgcacagatgcgtaa
ggagaaaataccgcatcaggcgccattcgccattcaggctgcgcaactgt
tgggaagggcgatcggtgcgggcctcttcgctattacgccagctggcgaa
agggggatgtgctgcaaggcgattaagttgggtaacgccagggttttccc
agtcacgacgttgtaaaacgacggccagtgccaagctg
[SEQ ID No: 22]

Preferably, the genetic construct comprises a nucleic acid sequence substantially as set out in SEQ ID No: 22, or a fragment or variant thereof.

Another embodiment of the genetic construct comprising a Bax encoding sequence is shown in FIG. 8I and is represented by SEQ ID No: 79, as follows:

acgcgtgtagtcttatgcaatactcttgtagtcttgcaacatggtaacga
tgagttagcaacatgccttacaaggagagaaaaagcaccgtgcatgccga
ttggtggaagtaaggtggtacgatcgtgccttattaggaaggcaacagac
gggtctgacatggattggacgaaccactgaattgccgcattgcagagata
ttgtatttaagtgcctagctcgatacaataaacgggtctctctggttaga
ccagatctgagcctgggagctctctggctaactagggaacccactgctta
agcctcaataaagcttgccttgagtgcttcaagtagtgtgtgcccgtctg
ttgtgtgactctggtaactagagatccctcagacccttttagtcagtgtg
gaaaatctctagcagtggcgcccgaacagggacctgaaagcgaaagggaa
accagagctctctcgacgcaggactcggcttgctgaagcgcgcacggcaa
gaggcgaggggcggcgactggtgagtacgccaaaaattttgactagcgga
ggctagaaggagagagatgggtgcgagagcgtcagtattaagcgggggag
aattagatcgcgatgggaaaaaattcggttaaggccagggggaaagaaaa
aatataaattaaaacatatagtatgggcaagcagggagctagaacgattc
gcagttaatcctggcctgttagaaacatcagaaggctgtagacaaatact
gggacagctacaaccatcccttcagacaggatcagaagaacttagatcat
tatataatacagtagcaaccctctattgtgtgcatcaaaggatagagata
aaagacaccaaggaagctttagacaagatagaggaagagcaaaacaaaag
taagaccaccgcacagcaagcggccactgatcttcagacctggaggagga
gatatgagggacaattggagaagtgaattatataaatataaagtagtaaa
aattgaaccattaggagtagcacccaccaaggcaaagagaagagtggtgc
agagagaaaaaagagcagtgggaataggagctttgttccttgggttcttg
ggagcagcaggaagcactatgggcgcagcctcaatgacgctgacggtaca
ggccagacaattattgtctggtatagtgcagcagcagaacaatttgctga
gggctattgaggcgcaacagcatctgttgcaactcacagtctggggcatc
aagcagctccaggcaagaatcctggctgtggaaagatacctaaaggatca
acagctcctggggatttggggttgctctggaaaactcatttgcaccactg
ctgtgccttggaatgctagttggagtaataaatctctggaacagattTgg
aatcacacgacctggatggagtgggacagagaaattaacaattacacaag
cttaatacactccttaattgaagaatcgcaaaaccagcaagaaaagaatg
aacaagaattattggaattagataaatgggcaagtttgtggaattggttt
aacataacaaattggctgtggtatataaaattattcataatgatagtagg
aggcttggtaggtttaagaatagtttttgctgtactttctatagtgaata
gagttaggcagggatattcaccattatcgtttcagacccacctcccaacc
ccgaggggacccgacaggcccgaaggaatagaagaagaaggtggagagag
agacagagacagatccattcgattagtgaacggatctcgacggtATCGGT
taacttttaaaagaaaaggggggattggggggtacagtgcaggggaaaga
atagtagacataatagcaacagacatacaaactaaagaattacaaaaaca
aattacaaaaattcaaaattttatGTCGAGccatagagcccaccgcatcc
ccagcatgcctgctattgtcttcccaatcctcccccttgctgtcctgccc
caccccaccccccagaatagaatgacacctactcagacaatgcgatgcaa
tttcctcattttattaggaaaggacagtgggagtggcaccttccagggtc
aaggaaggcacgggggaggggcaaacaacagatggctggcaactagaagg
cacagtcgCtcgagGGGCCTGTCCGCAAGCACGCTGCATAGCGGGCCTGT
CCGCAAGCACGCTGCACACTGGGCCTGTCCGCAAGCACGCTGCATAGCGG
GCCTGTCCGCAAGCACGCTGCACACTCCCACCCCTAAACAACGTAATGTA
GCCCCACCCCTAAACAACGTAATGCACTCCCACCCCTAAACAACGTAATG
TAGCCCCACCCCTAAACAACGTAATGCACTTGACATCATACCCGTGAAGG
TCTAGCTGACATCATACCCGTGAAGGTCCACTTGACATCATACCCGTGAA
GGTCTAGCTGACATCATACCCGTGAAGGTCCACTACTCCGCCCCCCCGCT
CGTAGCACTCCGCCCCCCCGCTCGCACTACTCCGCCCCCCCGCTCGTAGC
ACTCCGCCCCCCCGCTCGCACTGACGTTACATTCGTGAAGAATGTAGCGA
CGTTACATTCGTGAAGAATGCACTGACGTTACATTCGTGAAGAATGTAGC
GACGTTACATTCGTGAAGAATGCACTGCCCCAGCCGCCGCTGCACTAGCG
CCCCAGCCGCCGCTGCACCACTGCCCCAGCCGCCGCTGCACTAGCGCCCC
AGCCGCCGCTGCACctcgagTCAAACCTTCCTCTTCTTCTTAGGAGGCCT
CAGGCTGCTCTGCCCGCTTTCCAGTCGGGAAACCTGTCGTGCCAGCTGCA
TTAATGAATCGGCCAACGCGCGGGGAGAGGCGGTTTGCGTATTGGGCGCC
AGGGTGGTTTTTCTTTTCACCAGTGAGACGGGCAACAGCTGATTGCCCTT
CACCGCCTGGCCCTGAGAGAGTTGCAGCAAGCGGTCCACGCTGGTTTGCC
CCAGCAGGCGAAAATCCTGTTTGATGGTGGTTAACGGCGGGATATAACAT
GAGCTGTCTTCGGTATCGTCGTATCCCACTACCGAGATATCCGCACCAAC
GCGCAGCCCGGACTCGGTAATGGCGCGCATTGCGCCCAGCGCCATCTGAT
CGTTGGCAACCAGCATCGCAGTGGGAACGATGCCCTCATTCAGCATTTGC
ATGGTTTGTTGAAAACCGGACATGGCACTCCAGTCGCCTTCCCGTTCCGC
TATCGGCTGAATTTGATTGCGAGTGAGATATTTATGCCAGCCAGCCAGAC
GCAGACGCGCCGAGACAGAACTTAATGGGCCCGCTAACAGCGCGATTTGC
TGGTGACCCAATGCGACCAGATGCTCCACGCCCAGTCGCGTACCGTCTTC
ATGGGAGAAAATAATACTGTTGATGGGTGTCTGGTCAGAGACATCAAGAA
ATAACGCCGGAACATTAGTGCAGGCAGCTTCCACAGCAATGGCATCCTGG
TCATCCAGCGGATAGTTAATGATCAGCCCACTGACGCGTTGCGCGAGAAG
ATTGTGCACCGCCGCTTTACAGGCTTCGACGCCGCTTCGTTCTACCATCG
ACACCACCACGCTGGCACCCAGTTGATCGGCGCGAGATTTAATCGCCGCG
ACAATTTGCGACGGCGCGTGCAGGGCCAGACTGGAGGTGGCAACGCCAAT
CAGCAACGACTGTTTGCCCGCCAGTTGTTGTGCCACGCGGTTGGGAATGT
AATTCAGCTCCGCCATCGCCGCTTCCACTTTTTCCCGCGTTTTCGCAGAA
ACGTGGCTGGCCTGGTTCACCACGCGGGAAACGGTCTGATAAGAGACACC
GGCATACTCTGCGACATCGTATAACGTTACTGGTTTCATGGTGGGAGGGT
ACcTAGGTAAGCTTGGGCTGCAGGTCGAAAGGCCCGGAGATGAGGAAGAG
GAGAACAGCGCGGCAGACGTGCGCTTTTGAAGCGTGCAGAATGCCGGGCC
TCCGGAGGACCTTCGGGCGCCCGCCCCGCCCCTGAGCCCGCCCCTGAGCC
CGCCCCCGGACCCACCCCTTCCCAGCCTCTGAGCCCAGAAAGCGAAGGAG
CAAAGCTGCTATTGGCCGCTGCCCCAAAGGCCTACCCGCTTCCATTGCTC
AGCGGTGCTGTCCATCTGCACGAGACTAGTGAGACGTGCTACTTCCATTT
GTCACGTCCTGCACGACGCGAGCTGCGGGGCGGGGGGGAACTTCCTGACT
AGGGGAGGAGTAGAAGGTGGCGCGAAGGGGCCACCAAAGAACGGAGCCGG
TTGGCGCCTACCGGTGGATGTGGAATGTGTGCGAGGCCAGAGGCCACTTG
TGTAGCGCCAAGTGCCCAGCGGGGCTGCTAAAGCGCATGCTCCAGACTGC
CTTGGGAAAAGCGCCTCCCCTACCCGGTAGAAGCTAGCGGCTCCGGTGCC
CGTCAGTGGGCAGAGCGCACATCGCCCACAGTCCCCGAGAAGTTGTGGGG
AGGGGTCGGCAATTGAACCGGTGCCTAGAGAAGGTGGCGCGGGGTAAACT
GGGAAAGTGATGTCGTGTACTGGCTCCGCCTTTTTCCCGAGGGTGGGGGA
GAACCGTATATAAGTGCAGTAGTCGCCGTGAACGTTCTTTTTCGCAACGG
GTTTGCCGCCAGAACACAGGTAAGTGCCGTGTGTGGTTCCCGCGGGCCTG
GCCTCTTTACGGGTTATGGCCCTTGCGTGCCTTGAATTACTTCCACCTGG
CTGCAGTACGTGATTCTTGATCCCGAGCTTCGGGTTGGAAGTGGGTGGGA
GAGTTCGAGGCCTTGCGCTTAAGGAGCCCCTTCGCCTCGTGCTTGAGTTG
AGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGG
GCCAAGATCTGCACACTGGTATTTCGGTTTTTGGGGCCGCGGGCGGCGAC
GGGGCCCGTGCGTCCCAGCGCACATGTTCGGCGAGGCGGGGCCTGCGAGC
GCGGCCACCGAGAATCGGACGGGGGTAGTCTCAAGCTGGCCGGCCTGCTC
TGGTGCCTGGCCTCGCGCCGCCGTGTATCGCCCCGCCCTGGGCGGCAAGG
CTGGCCCGGTCGGCACCAGTTGCGTGAGCGGAAAGATGGCCGCTTCCCGG
CCCTGCTGCAGGGAGCTCAAAATGGAGGACGCGGCGCTCGGGAGAGCGGG
CGGGTGAGTCACCCACACAAAGGAAAAGGGCCTTTCCGTCCTCAGCCGTC
GCTTCATGTGACTCCACGGAGTACCGGGCGCCGTCCAGGCACCTCGATTA
GTTCTCGACCTTTTGGAGTACGTCGTCTTTAGGTTGGGGGGAGGGGTTTT
ATGCGATGGAGTTTCCCCACACTGAGTGGGTGGAGACTGAAGTTAGGCCA
GCTTGGCACTTGATGTAATTCTCCTTGGAATTTGCCCTTTTTGAGTTTGG
ATCTTGGTTCATTCTCAAGCCTCAGACAGTGGTTCAAAGTTTTTTTCTTC
CATTTCAGGTGTCGTGAtctagagctagcgAATTCGTAAATATAAAATTT
ACTAGGTTGTGGAATTGTGAGCGCTCACAATTCCACAGTCGACCCTAGGT
TGTGGAATTGTGAGCGCTCACAATTCCACAGTCGACCCTAGGTTGTGGAA
TTGTGAGCGCTCACAATTCCACAGTCGACCCTAGTGTATAATGTGTTAAA
CTACGGATCCGTCTCCCATTAGGCCTACAATGGTGAGACAAGTAGCCAAC
AGGGAAGGGTTGCAAATATCATTTGGGCACACCTATGATAATATTGATGA
AGCAGACAGTATTCAGCAAGTAACTGAGAGGTGGGAAGCTCAAAGCCAAA
GTCCTAATGTGCAGTCAGGTGAATTTATTGAAAAATTTGAGGCTCCTGGT
GGTGCAAATCAAAGAACTGCTCCTCAGGGATCCTAATTGTTTGTGTATTT
TAGGCggccgctgaatggacgggtccggggagcagcccagaggcgggggg
cccaccagctctgagcagatcatgaagacaggggcccttttgcttcaggg
tttcatccaggatcgagcagggcgaatggggggggaggcacccgagctgg
ccctggacccggtgcctcaggatgcgtccaccaagaagctgagcgagtgt
ctcaagcgcatcggggacgaactggacagtaacatggagctgcagaggat
gattgccgccgtggacacagactccccccgagaggtctttttccgagtgg
cagctgacatgttttctgacggcaacttcaactggggccgggttgtcgcc
cttttctactttgccagcaaactggtgctcaaggccctgtgcaccaaggt
gccggaactgatcagaaccatcatgggctggacattggacttcctccggg
agcggctgttgggctggatccaagaccagggtggttgggggctgcccctg
gccgagtcactgaagcgactgatgtccctgtctccaggacggcctcctct
cctactttgggacgcccacgtggcagaccgtgaccatctttgtggcggga
gtgctcaccgcctcactcaccatctggaagaagatgggctgaggccccca
gctgccttggactgtgtttttcctccagcggccgctgagggcagaggaag
tcttctaacatgcggtgacgtggaggagaatcccggcccttccggaATGG
AGAGCGACGAGAGCGGCCTGCCCGCCATGGAGATCGAGTGCCGCATCACC
GGCACCCTGAACGGCGTGGAGTTCGAGCTGGTGGGCGGCGGAGAGGGCAC
CCCCAAGCAGGGCCGCATGACCAACAAGATGAAGAGCACCAAAGGCGCCC
TGACCTTCAGCCCCTACCTGCTGAGCCACGTGATGGGCTACGGCTTCTAC
CACTTCGGCACCTACCCCAGCGGCTACGAGAACCCCTTCCTGCACGCCAT
CAACAACGGCGGCTACACCAACACCCGCATCGAGAAGTACGAGGACGGCG
GCGTGCTGCACGTGAGCTTCAGCTACCGCTACGAGGCCGGCCGCGTGATC
GGCGACTTCAAGGTGGTGGGCACCGGCTTCCCCGAGGACAGCGTGATCTT
CACCGACAAGATCATCCGCAGCAACGCCACCGTGGAGCACCTGCACCCCA
TGGGCGATAACGTGCTGGTGGGCAGCTTCGCCCGCACCTTCAGCCTGCGC
GACGGCGGCTACTACAGCTTCGTGGTGGACAGCCACATGCACTTCAAGAG
CGCCATCCACCCCAGCATCCTGCAGAACGGGGGCCCCATGTTCGCCTTCC
GCCGCGTGGAGGAGCTGCACAGCAACACCGAGCTGGGCATCGTGGAGTAC
CAGCACGCCTTCAAGACCCCCATCGCCTTCGCCAGATCCCGCGCTCAGTC
GTCCAATTCTGCCGTGGACGGCACCGCCGGACCCGGCTCCACCGGATCTC
GCTAGAGCTGAATCtaagtcgacaatcaacctctggattacaaaatttgt
gaaagattgactggtattcttaactatgttgctccttttacgctatgtgg
atacgctgctttaatgcctttgtatcatgctattgcttcccgtatggctt
tcattttctcctccttgtataaatcctggttgctgtctctttatgaggag
ttgtggcccgttgtcaggcaacgtggcgtggtgtgcactgtgtttgctga
cgcaacccccactggttggggcattgccaccacctgtcagctcctttccg
ggactttcgctttccccctccctattgccacggcggaactcatcgccgcc
tgccttgcccgctgctggacaggggctcggctgttgggcactgacaattc
cgtggtgttgtcggggaaatcatcgtcctttccttggctgctcgcctgtg
ttgccacctggattctgcgcgggacgtccttctgctacgtcccttcggcc
ctcaatccagcggaccttccttcccgcggcctgctgccggctctgcggcc
tcttccgcgtcttcgccttcgccctcagacgagtcggatctccctttggg
ccgcctccccgcccgggACACACCAAGGATAATTTCTCCCGATACACACC
AAGGATAATTTCTCCTCACACACACCAAGGATAATTTCTCCCGATACACA
CCAAGGATAATTTCTCCTCACAACTGGATGTCCCTGTATGATTCGATAAC
TGGATGTCCCTGTATGATTTCACAACTGGATGTCCCTGTATGATTCGATA
ACTGGATGTCCCTGTATGATTTCACAAAGAGGTTAACCATGTATTATCGA
TAAAGAGGTTAACCATGTATTATTCACAAAGAGGTTAACCATGTATTATC
GATAAAGAGGTTAACCATGTATTATTCACGGTTAGTGGACCGTGTTACAT
ACGATGGTTAGTGGACCGTGTTACATATCACGGTTAGTGGACCGTGTTAC
ATACGATGGTTAGTGGACCGTGTTACATATCACGAATTCACCAAGGGCAA
CCTCTCGATGAATTCACCAAGGGCAACCTCTTCACGAATTCACCAAGGGC
AACCTCTCGATGAATTCACCAAGGGCAACCTCTTCACAACTCCTGTATGA
AGCCGTTCCGATAACTCCTGTATGAAGCCGTTCTCACAACTCCTGTATGA
AGCCGTTCCGATAACTCCTGTATGAAGCCGTTCTCACATGGCAATATGTT
GGCATAGCACGATATGGCAATATGTTGGCATAGCATCACATGGCAATATG
TTGGCATAGCACGATATGGCAATATGTTGGCATAGCATCACGCACAGCAA
GTGTAGACAGGCACGATGCACAGCAAGTGTAGACAGGCATCACGCACAGC
AAGTGTAGACAGGCACGATGCACAGCAAGTGTAGACAGGCATCACAACAT
ACAAAGGGTATCCTCTCGATAACATACAAAGGGTATCCTCTTCACAACAT
ACAAAGGGTATCCTCTCGATAACATACAAAGGGTATCCTCTTCACCTCCC
TTCTTTCCTCCCGTCTTCGATCTCCCTTCTTTCCTCCCGTCTTTCACCTC
CCTTCTTTCCTCCCGTCTTCGATCTCCCTTCTTTCCTCCCGTCTTcccgg
gtacctttaagaccaatgacttacaaggcagctgtagatcttagccactt
tttaaaagaaaaggggggactggaagggctaattcactcccaacgaaaat
aagatctgctttttgcttgtactgggtctctctggttagaccagatctga
gcctgggagctctctggctaactagggaacccactgcttaagcctcaata
aagcttgccttgagtgcttcaagtagtgtgtgcccgtctgttgtgtgact
ctggtaactagagatccctcagacccttttagtcagtgtggaaaatctct
agcagtagtagttcatgtcatcttattattcagtatttataacttgcaaa
gaaatgaatatcagagagtgagaggaacttgtttattgcagcttataatg
gttacaaataaagcaatagcatcacaaatttcacaaataaagcatttttt
tcactgcattctagttgtggtttgtccaaactcatcaatgtatcttatca
tgtctggctctagctatcccgcccctaactccgcccagttccgcccattc
tccgccccatggctgactaattttttttatttatgcagaggccgaggccg
cctcggcctctgagctattccagaagtagtgaggaggcttttttggaggc
ctagacttttgcagagacggcccaaattcgtaatcatggtcatagctgtt
tcctgtgtgaaattgttatccgctcacaattccacacaacatacgagccg
gaagcataaagtgtaaagcctggggtgcctaatgagtgagctaactcaca
ttaattgcgttgcgctcactgcccgctttccagtcgggaaacctgtcgtg
ccagctgcattaatgaatcggccaacgcgcggggagaggcggtttgcgta
ttgggcgctcttccgcttcctcgctcactgactcgctgcgctcggtcgtt
cggctgcggcgagcggtatcagctcactcaaaggcggtaatacggttatc
cacagaatcaggggataacgcaggaaagaacatgtgagcaaaaggccagc
aaaaggccaggaaccgtaaaaaggccgcgttgctggcgtttttccatagg
ctccgcccccctgacgagcatcacaaaaatcgacgctcaagtcagaggtg
gcgaaacccgacaggactataaagataccaggcgtttccccctggaagct
ccctcgtgcgctctcctgttccgaccctgccgcttaccggatacctgtcc
gcctttctcccttcgggaagcgtggcgctttctcatagctcacgctgtag
gtatctcagttcggtgtaggtcgttcgctccaagctgggctgtgtgcacg
aaccccccgttcagcccgaccgctgcgccttatccggtaactatcgtctt
gagtccaacccggtaagacacgacttatcgccactggcagcagccactgg
taacaggattagcagagcgaggtatgtaggcggtgctacagagttcttga
agtggtggcctaactacggctacactagaaggacagtatttggtatctgc
gctctgctgaagccagttaccttcggaaaaagagttggtagctcttgatc
cggcaaacaaaccaccgctggtagcggtggtttttttgtttgcaagcagc
agattacgcgcagaaaaaaaggatctcaagaagatcctttgatcttttct
acggggtctgacgctcagtggaacgaaaactcacgttaagggattttggt
catgagattatcaaaaaggatcttcacctagatccttttaaattaaaaat
gaagttttaaatcaatctaaagtatatatgagtaaacttggtctgacagt
taccaatgcttaatcagtgaggcacctatctcagcgatctgtctatttcg
ttcatccatagttgcctgactccccgtcgtgtagataactacgatacggg
agggcttaccatctggccccagtgctgcaatgataccgcgagacccacgc
tcaccggctccagatttatcagcaataaaccagccagccggaagggccga
gcgcagaagtggtcctgcaactttatccgcctccatccagtctattaatt
gttgccgggaagctagagtaagtagttcgccagttaatagtttgcgcaac
gttgttgccattgctacaggcatcgtggtgtcacgctcgtcgtttggtat
ggcttcattcagctccggttcccaacgatcaaggcgagttacatgatccc
ccatgttgtgcaaaaaagcggttagctccttcggtcctccgatcgttgtc
agaagtaagttggccgcagtgttatcactcatggttatggcagcactgca
taattctcttactgtcatgccatccgtaagatgcttttctgtgactggtg
agtactcaaccaagtcattctgagaatagtgtatgcggcgaccgagttgc
tcttgcccggcgtcaatacgggataataccgcgccacatagcagaacttt
aaaagtgctcatcattggaaaacgttcttcggggcgaaaactctcaagga
tcttaccgctgttgagatccagttcgatgtaacccactcgtgcacccaac
tgatcttcagcatcttttactttcaccagcgtttctgggtgagcaaaaac
aggaaggcaaaatgccgcaaaaaagggaataagggcgacacggaaatgtt
gaatactcatactcttcctttttcaatattattgaagcatttatcagggt
tattgtctcatgagcggatacatatttgaatgtatttagaaaaataaaca
aataggggttccgcgcacatttccccgaaaagtgccacctgacgtctaag
aaaccattattatcatgacattaacctataaaaataggcgtatcacgagg
ccctttcgtctcgcgcgtttcggtgatgacggtgaaaacctctgacacat
gcagctcccggagacggtcacagcttgtctgtaagcggatgccgggagca
gacaagcccgtcagggcgcgtcagcgggtgttggcgggtgtcggggctgg
cttaactatgcggcatcagagcagattgtactgagagtgcaccatatgcg
gtgtgaaataccgcacagatgcgtaaggagaaaataccgcatcaggcgcc
attcgccattcaggctgcgcaactgttgggaagggcgatcggtgcgggcc
tcttcgctattacgccagctggcgaaagggggatgtgctgcaaggcgatt
aagttgggtaacgccagggttttcccagtcacgacgttgtaaaacgacgg
ccagtgccaagctg
[SEQ ID No: 79]

Preferably, the genetic construct comprises a nucleic acid sequence substantially as set out in SEQ ID No: 79, or a fragment or variant thereof.

Another embodiment of the genetic construct is shown in FIG. 8E and may comprise an Apoptin encoding sequence and be represented by SEQ ID No: 23, as follows:

acgcgtgtagtcttatgcaatactcttgtagtcttgcaacatggtaacga
tgagttagcaacatgccttacaaggagagaaaaagcaccgtgcatgccga
ttggtggaagtaaggtggtacgatcgtgccttattaggaaggcaacagac
gggtctgacatggattggacgaaccactgaattgccgcattgcagagata
ttgtatttaagtgcctagctcgatacaataaacgggtctctctggttaga
ccagatctgagcctgggagctctctggctaactagggaacccactgctta
agcctcaataaagcttgccttgagtgcttcaagtagtgtgtgcccgtctg
ttgtgtgactctggtaactagagatccctcagacccttttagtcagtgtg
gaaaatctctagcagtggcgcccgaacagggacctgaaagcgaaagggaa
accagagctctctcgacgcaggactcggcttgctgaagcgcgcacggcaa
gaggcgaggggcggcgactggtgagtacgccaaaaattttgactagcgga
ggctagaaggagagagatgggtgcgagagcgtcagtattaagcgggggag
aattagatcgcgatgggaaaaaattcggttaaggccagggggaaagaaaa
aatataaattaaaacatatagtatgggcaagcagggagctagaacgattc
gcagttaatcctggcctgttagaaacatcagaaggctgtagacaaatact
gggacagctacaaccatcccttcagacaggatcagaagaacttagatcat
tatataatacagtagcaaccctctattgtgtgcatcaaaggatagagata
aaagacaccaaggaagctttagacaagatagaggaagagcaaaacaaaag
taagaccaccgcacagcaagcggccactgatcttcagacctggaggagga
gatatgagggacaattggagaagtgaattatataaatataaagtagtaaa
aattgaaccattaggagtagcacccaccaaggcaaagagaagagtggtgc
agagagaaaaaagagcagtgggaataggagctttgttccttgggttcttg
ggagcagcaggaagcactatgggcgcagcctcaatgacgctgacggtaca
ggccagacaattattgtctggtatagtgcagcagcagaacaatttgctga
gggctattgaggcgcaacagcatctgttgcaactcacagtctggggcatc
aagcagctccaggcaagaatcctggctgtggaaagatacctaaaggatca
acagctcctggggatttggggttgctctggaaaactcatttgcaccactg
ctgtgccttggaatgctagttggagtaataaatctctggaacagattTgg
aatcacacgacctggatggagtgggacagagaaattaacaattacacaag
cttaatacactccttaattgaagaatcgcaaaaccagcaagaaaagaatg
aacaagaattattggaattagataaatgggcaagtttgtggaattggttt
aacataacaaattggctgtggtatataaaattattcataatgatagtagg
aggcttggtaggtttaagaatagtttttgctgtactttctatagtgaata
gagttaggcagggatattcaccattatcgtttcagacccacctcccaacc
ccgaggggacccgacaggcccgaaggaatagaagaagaaggtggagagag
agacagagacagatccattcgattagtgaacggatctcgacggtATCGGT
taacttttaaaagaaaaggggggattggggggtacagtgcaggggaaaga
atagtagacataatagcaacagacatacaaactaaagaattacaaaaaca
aattacaaaaattcaaaattttatGTCGAGccatagagcccaccgcatcc
ccagcatgcctgctattgtcttcccaatcctcccccttgctgtcctgccc
caccccaccccccagaatagaatgacacctactcagacaatgcgatgcaa
tttcctcattttattaggaaaggacagtgggagtggcaccttccagggtc
aaggaaggcacgggggaggggcaaacaacagatggctggcaactagaagg
cacagtcgCtcgagAGTAAAACACTACAACGTCGATAGCAGTAAAACACT
ACAACGTCGACACTAGTAAAACACTACAACGTCGATAGCAGTAAAACACT
ACAACGTCGACACTAGTGTCACTTGGCCAGAGAAATAGCAGTGTCACTTG
GCCAGAGAAACACTAGTGTCACTTGGCCAGAGAAATAGCAGTGTCACTTG
GCCAGAGAAActcgagTCAAACCTTCCTCTTCTTCTTAGGAGGCCTCAGG
CTGCTCTGCCCGCTTTCCAGTCGGGAAACCTGTCGTGCCAGCTGCATTAA
TGAATCGGCCAACGCGCGGGGAGAGGCGGTTTGCGTATTGGGCGCCAGGG
TGGTTTTTCTTTTCACCAGTGAGACGGGCAACAGCTGATTGCCCTTCACC
GCCTGGCCCTGAGAGAGTTGCAGCAAGCGGTCCACGCTGGTTTGCCCCAG
CAGGCGAAAATCCTGTTTGATGGTGGTTAACGGCGGGATATAACATGAGC
TGTCTTCGGTATCGTCGTATCCCACTACCGAGATATCCGCACCAACGCGC
AGCCCGGACTCGGTAATGGCGCGCATTGCGCCCAGCGCCATCTGATCGTT
GGCAACCAGCATCGCAGTGGGAACGATGCCCTCATTCAGCATTTGCATGG
TTTGTTGAAAACCGGACATGGCACTCCAGTCGCCTTCCCGTTCCGCTATC
GGCTGAATTTGATTGCGAGTGAGATATTTATGCCAGCCAGCCAGACGCAG
ACGCGCCGAGACAGAACTTAATGGGCCCGCTAACAGCGCGATTTGCTGGT
GACCCAATGCGACCAGATGCTCCACGCCCAGTCGCGTACCGTCTTCATGG
GAGAAAATAATACTGTTGATGGGTGTCTGGTCAGAGACATCAAGAAATAA
CGCCGGAACATTAGTGCAGGCAGCTTCCACAGCAATGGCATCCTGGTCAT
CCAGCGGATAGTTAATGATCAGCCCACTGACGCGTTGCGCGAGAAGATTG
TGCACCGCCGCTTTACAGGCTTCGACGCCGCTTCGTTCTACCATCGACAC
CACCACGCTGGCACCCAGTTGATCGGCGCGAGATTTAATCGCCGCGACAA
TTTGCGACGGCGCGTGCAGGGCCAGACTGGAGGTGGCAACGCCAATCAGC
AACGACTGTTTGCCCGCCAGTTGTTGTGCCACGCGGTTGGGAATGTAATT
CAGCTCCGCCATCGCCGCTTCCACTTTTTCCCGCGTTTTCGCAGAAACGT
GGCTGGCCTGGTTCACCACGCGGGAAACGGTCTGATAAGAGACACCGGCA
TACTCTGCGACATCGTATAACGTTACTGGTTTCATGGTGGGAGGGTACcT
AGGTAAGCTTGGGCTGCAGGTCGAAAGGCCCGGAGATGAGGAAGAGGAGA
ACAGCGCGGCAGACGTGCGCTTTTGAAGCGTGCAGAATGCCGGGCCTCCG
GAGGACCTTCGGGCGCCCGCCCCGCCCCTGAGCCCGCCCCTGAGCCCGCC
CCCGGACCCACCCCTTCCCAGCCTCTGAGCCCAGAAAGCGAAGGAGCAAA
GCTGCTATTGGCCGCTGCCCCAAAGGCCTACCCGCTTCCATTGCTCAGCG
GTGCTGTCCATCTGCACGAGACTAGTGAGACGTGCTACTTCCATTTGTCA
CGTCCTGCACGACGCGAGCTGCGGGGCGGGGGGGAACTTCCTGACTAGGG
GAGGAGTAGAAGGTGGCGCGAAGGGGCCACCAAAGAACGGAGCCGGTTGG
CGCCTACCGGTGGATGTGGAATGTGTGCGAGGCCAGAGGCCACTTGTGTA
GCGCCAAGTGCCCAGCGGGGCTGCTAAAGCGCATGCTCCAGACTGCCTTG
GGAAAAGCGCCTCCCCTACCCGGTAGAAGCTAGCGGCTCCGGTGCCCGTC
AGTGGGCAGAGCGCACATCGCCCACAGTCCCCGAGAAGTTGTGGGGAGGG
GTCGGCAATTGAACCGGTGCCTAGAGAAGGTGGCGCGGGGTAAACTGGGA
AAGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTTT
GCCGCCAGAACACAGGTAAGTGCCGTGTGTGGTTCCCGCGGGCCTGGCCT
CTTTACGGGTTATGGCCCTTGCGTGCCTTGAATTACTTCCACCTGGCTGC
AGTACGTGATTCTTGATCCCGAGCTTCGGGTTGGAAGTGGGTGGGAGAGT
TCGAGGCCTTGCGCTTAAGGAGCCCCTTCGCCTCGTGCTTGAGTTGAGGC
CTGGCCTGGGCGCTGGGGCCGCCGCGTGCGAATCTGGTGGCACCTTCGCG
CCTGTCTCGCTGCTTTCGATAAGTCTCTAGCCATTTAAAATTTTTGATGA
CCTGCTGCGACGCTTTTTTTCTGGCAAGATAGTCTTGTAAATGCGGGCCA
AGATCTGCACACTGGTATTTCGGTTTTTGGGGCCGCGGGCGGCGACGGGG
CCCGTGCGTCCCAGCGCACATGTTCGGCGAGGCGGGGCCTGCGAGCGCGG
CCACCGAGAATCGGACGGGGGTAGTCTCAAGCTGGCCGGCCTGCTCTGGT
GCCTGGCCTCGCGCCGCCGTGTATCGCCCCGCCCTGGGCGGCAAGGCTGG
CCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGG
TGAGTCACCCACACAAAGGAAAAGGGCCTTTCCGTCCTCAGCCGTCGCTT
CATGTGACTCCACGGAGTACCGGGCGCCGTCCAGGCACCTCGATTAGTTC
TCGACCTTTTGGAGTACGTCGTCTTTAGGTTGGGGGGAGGGGTTTTATGC
GATGGAGTTTCCCCACACTGAGTGGGTGGAGACTGAAGTTAGGCCAGCTT
GGCACTTGATGTAATTCTCCTTGGAATTTGCCCTTTTTGAGTTTGGATCT
TGGTTCATTCTCAAGCCTCAGACAGTGGTTCAAAGTTTTTTTCTTCCATT
TCAGGTGTCGTGAtctagagctagcgAATTCGTAAATATAAAATTTACTA
GGTTGTGGAATTGTGAGCGCTCACAATTCCACAGTCGACCCTAGGTTGTG
GAATTGTGAGCGCTCACAATTCCACAGTCGACCCTAGGTTGTGGAATTGT
GAGCGCTCACAATTCCACAGTCGACCCTAGTGTATAATGTGTTAAACTAC
GGATCCGTCTCCCATTAGGCCTACAATGGTGAGACAAGTAGCCAACAGGG
AAGGGTTGCAAATATCATTTGGGCACACCTATGATAATATTGATGAAGCA
GACAGTATTCAGCAAGTAACTGAGAGGTGGGAAGCTCAAAGCCAAAGTCC
TAATGTGCAGTCAGGTGAATTTATTGAAAAATTTGAGGCTCCTGGTGGTG
CAAATCAAAGAACTGCTCCTCAGGGATCCTAATTGTTTGTGTATTTTAGG
Cggccgcatgaacgctctccaagaagatactccacccggaccatcaacgg
tgttcaggccaccaacaagttcacggccgttggaaacccctcactgcaga
gagatccggattggtatcgctggaattacaatcactctatcgctgtgtgg
ctgcgcgaatgctcgcgctcccacgctaagatctgcaactgcggacaatt
cagaaagcactggtttcaagaatgtgccggacttgaggaccgatcaaccc
aagcctccctcgaagaagcgatcctgcgacccctccgagtacagggtaag
cgagctaaaagaaagcttgattaccactactcccagccgaccccgaaccg
caagaaggcgtataagactgtaaGCGGCCGCtgagggcagaggaagtctt
ctaacatgcggtgacgtggaggagaatcccggcccttccggaATGGAGAG
CGACGAGAGCGGCCTGCCCGCCATGGAGATCGAGTGCCGCATCACCGGCA
CCCTGAACGGCGTGGAGTTCGAGCTGGTGGGCGGCGGAGAGGGCACCCCC
AAGCAGGGCCGCATGACCAACAAGATGAAGAGCACCAAAGGCGCCCTGAC
CTTCAGCCCCTACCTGCTGAGCCACGTGATGGGCTACGGCTTCTACCACT
TCGGCACCTACCCCAGCGGCTACGAGAACCCCTTCCTGCACGCCATCAAC
AACGGCGGCTACACCAACACCCGCATCGAGAAGTACGAGGACGGCGGCGT
GCTGCACGTGAGCTTCAGCTACCGCTACGAGGCCGGCCGCGTGATCGGCG
ACTTCAAGGTGGTGGGCACCGGCTTCCCCGAGGACAGCGTGATCTTCACC
GACAAGATCATCCGCAGCAACGCCACCGTGGAGCACCTGCACCCCATGGG
CGATAACGTGCTGGTGGGCAGCTTCGCCCGCACCTTCAGCCTGCGCGACG
GCGGCTACTACAGCTTCGTGGTGGACAGCCACATGCACTTCAAGAGCGCC
ATCCACCCCAGCATCCTGCAGAACGGGGGCCCCATGTTCGCCTTCCGCCG
CGTGGAGGAGCTGCACAGCAACACCGAGCTGGGCATCGTGGAGTACCAGC
ACGCCTTCAAGACCCCCATCGCCTTCGCCAGATCCCGCGCTCAGTCGTCC
AATTCTGCCGTGGACGGCACCGCCGGACCCGGCTCCACCGGATCTCGCTA
GAGCTGAATCtaagtcgacaatcaacctctggattacaaaatttgtgaaa
gattgactggtattcttaactatgttgctccttttacgctatgtggatac
gctgctttaatgcctttgtatcatgctattgcttcccgtatggctttcat
tttctcctccttgtataaatcctggttgctgtctctttatgaggagttgt
ggcccgttgtcaggcaacgtggcgtggtgtgcactgtgtttgctgacgca
acccccactggttggggcattgccaccacctgtcagctcctttccgggac
tttcgctttccccctccctattgccacggcggaactcatcgccgcctgcc
ttgcccgctgctggacaggggctcggctgttgggcactgacaattccgtg
gtgttgtcggggaaatcatcgtcctttccttggctgctcgcctgtgttgc
cacctggattctgcgcgggacgtccttctgctacgtcccttcggccctca
atccagcggaccttccttcccgcggcctgctgccggctctgcggcctctt
ccgcgtcttcgccttcgccctcagacgagtcggatctccctttgggccgc
ctccccgcccgggTAACCGATTTCAGATGGTGCTACGATTAACCGATTTC
AGATGGTGCTATCACTAACCGATTTCAGATGGTGCTACGATTAACCGATT
TCAGATGGTGCTATCACGGGAGTGAAGACACGGAGCCAGACGATGGGAGT
GAAGACACGGAGCCAGATCACGGGAGTGAAGACACGGAGCCAGACGATGG
GAGTGAAGACACGGAGCCAGAcccgggtacctttaagaccaatgacttac
aaggcagctgtagatcttagccactttttaaaagaaaaggggggactgga
agggctaattcactcccaacgaaaataagatctgctttttgcttgtactg
ggtctctctggttagaccagatctgagcctgggagctctctggctaacta
gggaacccactgcttaagcctcaataaagcttgccttgagtgcttcaagt
agtgtgtgcccgtctgttgtgtgactctggtaactagagatccctcagac
ccttttagtcagtgtggaaaatctctagcagtagtagttcatgtcatctt
attattcagtatttataacttgcaaagaaatgaatatcagagagtgagag
gaacttgtttattgcagcttataatggttacaaataaagcaatagcatca
caaatttcacaaataaagcatttttttcactgcattcagttgtggtttgt
ccaaactcatcaatgtatcttatcatgtctggctctagctateccgcccc
taactccgcccagttccgcccattctccgecccatggctgactaattttt
tttatttatgcagaggccgaggccgcctcggcctctgagctattccagaa
gtagtgaggaggcttttttggaggcctagacttttgcagagacggcccaa
attcgtaatcatggtcatagctgtttcctgtgtgaaattgttatccgctc
acaattccacacaacatacgagccggaagcataaagtgtaaagcctgggg
tgcctaatgagtgagctaactcacattaattgcgttgcgctcactgcccg
ctttccagtcgggaaacctgtcgtgccagctgcattaatgaatcggccaa
cgcgcggggagaggcggtttgcgtattgggcgctcttccgcttcctcgct
cactgactcgctgcgctcggtcgttcggctgcggcgagcggtatcagctc
actcaaaggcggtaatacggttatccacagaatcaggggataacgcagga
aagaacatgtgagcaaaaggccagcaaaaggccaggaaccgtaaaaaggc
cgcgttgctggcgtttttccataggctccgcccccctgacgagcatcaca
aaaatcgacgctcaagtcagaggtggcgaaacccgacaggactataaaga
taccaggcgtttccccctggaagctccctcgtgcgctctcctgttccgac
cctgccgcttaccggatacctgtccgcctttctcccttcgggaagcgtgg
cgctttctcatagctcacgctgtaggtatctcagttcggtgtaggtcgtt
cgctccaagctgggctgtgtgcacgaaccccccgttcagcccgaccgctg
cgccttatccggtaactatcgtcttgagtccaacccggtaagacacgact
tatcgccactggcagcagccactggtaacaggattagcagagcgaggtat
gtaggcggtgctacagagttcttgaagtggtggcctaactacggctacac
tagaaggacagtatttggtatctgcgctctgctgaagccagttaccttcg
gaaaaagagttggtagctcttgatccggcaaacaaaccaccgctggtagc
ggtggtttitttgtttgcaagcagcagattacgcgcagaaaaaaaggatc
tcaagaagatcctttgatcttttctacggggtctgacgctcagtggaacg
aaaactcacgttaagggattttggtcatgagattatcaaaaaggatcttc
acctagatccttttaaattaaaaatgaagttttaaatcaatctaaagtat
atatgagtaaacttggtctgacagttaccaatgcttaatcagtgaggcac
ctatctcagcgatctgtctatttcgttcatccatagttgcctgactcccc
gtcgtgtagataactacgatacgggagggcttaccatctggccccagtgc
tgcaatgataccgcgagacccacgctcaccggctccagatttatcagcaa
taaaccagccagccggaagggccgagcgcagaagtggtcctgcaacttta
tccgcctccatccagtctattaattgttgccgggaagctagagtaagtag
ttcgccagttaatagtttgcgcaacgttgttgccattgctacaggcatcg
tggtgtcacgctcgtcgtttggtatggcttcattcagctccggttcccaa
cgatcaaggcgagttacatgatcccccatgttgtgcaaaaaagcggttag
ctccttcggtcctccgatcgttgtcagaagtaagttggccgcagtgttat
cactcatggttatggcagcactgcataattctcttactgtcatgccatcc
gtaagatgcttttctgtgactggtgagtactcaaccaagtcattctgaga
atagtgtatgcggcgaccgagttgctcttgcccggcgtcaatacgggata
ataccgcgccacatagcagaactttaaaagtgctcatcattggaaaacgt
tcttcggggcgaaaactctcaaggatcttaccgctgttgagatccagttc
gatgtaacccactcgtgcacccaactgatcttcagcatcttttactttca
ccagcgtttctgggtgagcaaaaacaggaaggcaaaatgccgcaaaaaag
ggaataagggcgacacggaaatgttgaatactcatactcttcctttttca
atattattgaagcatttatcagggttattgtctcatgagcggatacatat
ttgaatgtatttagaaaaataaacaaataggggttccgcgcacatttccc
cgaaaagtgccacctgacgtctaagaaaccattattatcatgacattaac
ctataaaaataggcgtatcacgaggccctttcgtctcgcgcgtttcggtg
atgacggtgaaaacctctgacacatgcagctcccggagacggtcacagct
tgtctgtaagcggatgccgggagcagacaagcccgtcagggcgcgtcagc
gggtgttggcgggtgtcggggctggcttaactatgcggcatcagagcaga
ttgtactgagagtgcaccatatgcggtgtgaaataccgcacagatgcgta
aggagaaaataccgcatcaggcgccattcgccattcaggctgcgcaactg
ttgggaagggcgatcggtgcgggcctcttcgctattacgccagctggcga
aagggggatgtgctgcaaggcgattaagttgggtaacgccagggttttcc
cagtcacgacgttgtaaaacgacggccagtgccaagctg
[SEQ ID No: 23]

Preferably, the genetic construct comprises a nucleic acid sequence substantially as set out in SEQ ID No: 23, or a fragment or variant thereof.

Another embodiment of the genetic construct comprising an Apoptin encoding sequence is shown in FIG. 8J and is represented by SEQ ID No: 80, as follows:

acgcgtgtagtcttatgcaatactcttgtagtcttgcaacatggtaacga
tgagttagcaacatgccttacaaggagagaaaaagcaccgtgcatgccga
ttggtggaagtaaggtggtacgatcgtgccttattaggaaggcaacagac
gggtctgacatggattggacgaaccactgaattgccgcattgcagagata
ttgtatttaagtgcctagctcgatacaataaacgggtctctctggttaga
ccagatctgagcctgggagctctctggctaactagggaacccactgctta
agcctcaataaagcttgccttgagtgcttcaagtagtgtgtgcccgtctg
ttgtgtgactctggtaactagagatccctcagacccttttagtcagtgtg
gaaaatctctagcagtggcgcccgaacagggacctgaaagcgaaagggaa
accagagctctctcgacgcaggactcggcttgctgaagcgcgcacggcaa
gaggcgaggggcggcgactggtgagtacgccaaaaattttgactagcgga
ggctagaaggagagagatgggtgcgagagcgtcagtattaagcgggggag
aattagatcgcgatgggaaaaaattcggttaaggccagggggaaagaaaa
aatataaattaaaacatatagtatgggcaagcagggagctagaacgattc
gcagttaatcctggcctgttagaaacatcagaaggctgtagacaaatact
gggacagctacaaccatcccttcagacaggatcagaagaacttagatcat
tatataatacagtagcaaccctctattgtgtgcatcaaaggatagagata
aaagacaccaaggaagctttagacaagatagaggaagagcaaaacaaaag
taagaccaccgcacagcaagcggccactgatcttcagacctggaggagga
gatatgagggacaattggagaagtgaattatataaatataaagtagtaaa
aattgaaccattaggagtagcacccaccaaggcaaagagaagagtggtgc
agagagaaaaaagagcagtgggaataggagctttgttccttgggttcttg
ggagcagcaggaagcactatgggcgcagcctcaatgacgctgacggtaca
ggccagacaattattgtctggtatagtgcagcagcagaacaatttgctga
gggctattgaggcgcaacagcatctgttgcaactcacagtctggggcatc
aagcagctccaggcaagaatcctggctgtggaaagatacctaaaggatca
acagctcctggggatttggggttgctctggaaaactcatttgcaccactg
ctgtgccttggaatgctagttggagtaataaatctctggaacagattTgg
aatcacacgacctggatggagtgggacagagaaattaacaattacacaag
cttaatacactccttaattgaagaatcgcaaaaccagcaagaaaagaatg
aacaagaattattggaattagataaatgggcaagtttgtggaattggttt
aacataacaaattggctgtggtatataaaattattcataatgatagtagg
aggcttggtaggtttaagaatagtttttgctgtactttctatagtgaata
gagttaggcagggatattcaccattatcgtttcagacccacctcccaacc
ccgaggggacccgacaggcccgaaggaatagaagaagaaggtggagagag
agacagagacagatccattcgattagtgaacggatctcgacggtATCGGT
taacttttaaaagaaaaggggggattggggggtacagtgcaggggaaaga
atagtagacataatagcaacagacatacaaactaaagaattacaaaaaca
aattacaaaaattcaaaattttatGTCGAGccatagagcccaccgcatcc
ccagcatgcctgctattgtcttcccaatcctcccccttgctgtcctgccc
caccccaccccccagaatagaatgacacctactcagacaatgcgatgcaa
tttcctcattttattaggaaaggacagtgggagtggcaccttccagggtc
aaggaaggcacgggggaggggcaaacaacagatggctggcaactagaagg
cacagtcgCtcgagGGGCCTGTCCGCAAGCACGCTGCATAGCGGGCCTGT
CCGCAAGCACGCTGCACACTGGGCCTGTCCGCAAGCACGCTGCATAGCGG
GCCTGTCCGCAAGCACGCTGCACACTCCCACCCCTAAACAACGTAATGTA
GCCCCACCCCTAAACAACGTAATGCACTCCCACCCCTAAACAACGTAATG
TAGCCCCACCCCTAAACAACGTAATGCACTTGACATCATACCCGTGAAGG
TCTAGCTGACATCATACCCGTGAAGGTCCACTTGACATCATACCCGTGAA
GGTCTAGCTGACATCATACCCGTGAAGGTCCACTACTCCGCCCCCCCGCT
CGTAGCACTCCGCCCCCCCGCTCGCACTACTCCGCCCCCCCGCTCGTAGC
ACTCCGCCCCCCCGCTCGCACTGACGTTACATTCGTGAAGAATGTAGCGA
CGTTACATTCGTGAAGAATGCACTGACGTTACATTCGTGAAGAATGTAGC
GACGTTACATTCGTGAAGAATGCACTGCCCCAGCCGCCGCTGCACTAGCG
CCCCAGCCGCCGCTGCACCACTGCCCCAGCCGCCGCTGCACTAGCGCCCC
AGCCGCCGCTGCACctcgagTCAAACCTTCCTCTTCTTCTTAGGAGGCCT
CAGGCTGCTCTGCCCGCTTTCCAGTCGGGAAACCTGTCGTGCCAGCTGCA
TTAATGAATCGGCCAACGCGCGGGGAGAGGCGGTTTGCGTATTGGGCGCC
AGGGTGGTTTTTCTTTTCACCAGTGAGACGGGCAACAGCTGATTGCCCTT
CACCGCCTGGCCCTGAGAGAGTTGCAGCAAGCGGTCCACGCTGGTTTGCC
CCAGCAGGCGAAAATCCTGTTTGATGGTGGTTAACGGCGGGATATAACAT
GAGCTGTCTTCGGTATCGTCGTATCCCACTACCGAGATATCCGCACCAAC
GCGCAGCCCGGACTCGGTAATGGCGCGCATTGCGCCCAGCGCCATCTGAT
CGTTGGCAACCAGCATCGCAGTGGGAACGATGCCCTCATTCAGCATTTGC
ATGGTTTGTTGAAAACCGGACATGGCACTCCAGTCGCCTTCCCGTTCCGC
TATCGGCTGAATTTGATTGCGAGTGAGATATTTATGCCAGCCAGCCAGAC
GCAGACGCGCCGAGACAGAACTTAATGGGCCCGCTAACAGCGCGATTTGC
TGGTGACCCAATGCGACCAGATGCTCCACGCCCAGTCGCGTACCGTCTTC
ATGGGAGAAAATAATACTGTTGATGGGTGTCTGGTCAGAGACATCAAGAA
ATAACGCCGGAACATTAGTGCAGGCAGCTTCCACAGCAATGGCATCCTGG
TCATCCAGCGGATAGTTAATGATCAGCCCACTGACGCGTTGCGCGAGAAG
ATTGTGCACCGCCGCTTTACAGGCTTCGACGCCGCTTCGTTCTACCATCG
ACACCACCACGCTGGCACCCAGTTGATCGGCGCGAGATTTAATCGCCGCG
ACAATTTGCGACGGCGCGTGCAGGGCCAGACTGGAGGTGGCAACGCCAAT
CAGCAACGACTGTTTGCCCGCCAGTTGTTGTGCCACGCGGTTGGGAATGT
AATTCAGCTCCGCCATCGCCGCTTCCACTTTTTCCCGCGTTTTCGCAGAA
ACGTGGCTGGCCTGGTTCACCACGCGGGAAACGGTCTGATAAGAGACACC
GGCATACTCTGCGACATCGTATAACGTTACTGGTTTCATGGTGGGAGGGT
ACcTAGGTAAGCTTGGGCTGCAGGTCGAAAGGCCCGGAGATGAGGAAGAG
GAGAACAGCGCGGCAGACGTGCGCTTTTGAAGCGTGCAGAATGCCGGGCC
TCCGGAGGACCTTCGGGCGCCCGCCCCGCCCCTGAGCCCGCCCCTGAGCC
CGCCCCCGGACCCACCCCTTCCCAGCCTCTGAGCCCAGAAAGCGAAGGAG
CAAAGCTGCTATTGGCCGCTGCCCCAAAGGCCTACCCGCTTCCATTGCTC
AGCGGTGCTGTCCATCTGCACGAGACTAGTGAGACGTGCTACTTCCATTT
GTCACGTCCTGCACGACGCGAGCTGCGGGGCGGGGGGGAACTTCCTGACT
AGGGGAGGAGTAGAAGGTGGCGCGAAGGGGCCACCAAAGAACGGAGCCGG
TTGGCGCCTACCGGTGGATGTGGAATGTGTGCGAGGCCAGAGGCCACTTG
TGTAGCGCCAAGTGCCCAGCGGGGCTGCTAAAGCGCATGCTCCAGACTGC
CTTGGGAAAAGCGCCTCCCCTACCCGGTAGAAGCTAGCGGCTCCGGTGCC
CGTCAGTGGGCAGAGCGCACATCGCCCACAGTCCCCGAGAAGTTGTGGGG
AGGGGTCGGCAATTGAACCGGTGCCTAGAGAAGGTGGCGCGGGGTAAACT
GGGAAAGTGATGTCGTGTACTGGCTCCGCCTTTTTCCCGAGGGTGGGGGA
GAACCGTATATAAGTGCAGTAGTCGCCGTGAACGTTCTTTTTCGCAACGG
GTTTGCCGCCAGAACACAGGTAAGTGCCGTGTGTGGTTCCCGCGGGCCTG
GCCTCTTTACGGGTTATGGCCCTTGCGTGCCTTGAATTACTTCCACCTGG
CTGCAGTACGTGATTCTTGATCCCGAGCTTCGGGTTGGAAGTGGGTGGGA
GAGTTCGAGGCCTTGCGCTTAAGGAGCCCCTTCGCCTCGTGCTTGAGTTG
AGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGG
GCCAAGATCTGCACACTGGTATTTCGGTTTTTGGGGCCGCGGGCGGCGAC
GGGGCCCGTGCGTCCCAGCGCACATGTTCGGCGAGGCGGGGCCTGCGAGC
GCGGCCACCGAGAATCGGACGGGGGTAGTCTCAAGCTGGCCGGCCTGCTC
TGGTGCCTGGCCTCGCGCCGCCGTGTATCGCCCCGCCCTGGGCGGCAAGG
CTGGCCCGGTCGGCACCAGTTGCGTGAGCGGAAAGATGGCCGCTTCCCGG
CCCTGCTGCAGGGAGCTCAAAATGGAGGACGCGGCGCTCGGGAGAGCGGG
CGGGTGAGTCACCCACACAAAGGAAAAGGGCCTTTCCGTCCTCAGCCGTC
GCTTCATGTGACTCCACGGAGTACCGGGCGCCGTCCAGGCACCTCGATTA
GTTCTCGACCTTTTGGAGTACGTCGTCTTTAGGTTGGGGGGAGGGGTTTT
ATGCGATGGAGTTTCCCCACACTGAGTGGGTGGAGACTGAAGTTAGGCCA
GCTTGGCACTTGATGTAATTCTCCTTGGAATTTGCCCTTTTTGAGTTTGG
ATCTTGGTTCATTCTCAAGCCTCAGACAGTGGTTCAAAGTTTTTTTCTTC
CATTTCAGGTGTCGTGAtctagagctagcgAATTCGTAAATATAAAATTT
ACTAGGTTGTGGAATTGTGAGCGCTCACAATTCCACAGTCGACCCTAGGT
TGTGGAATTGTGAGCGCTCACAATTCCACAGTCGACCCTAGGTTGTGGAA
TTGTGAGCGCTCACAATTCCACAGTCGACCCTAGTGTATAATGTGTTAAA
CTACGGATCCGTCTCCCATTAGGCCTACAATGGTGAGACAAGTAGCCAAC
AGGGAAGGGTTGCAAATATCATTTGGGCACACCTATGATAATATTGATGA
AGCAGACAGTATTCAGCAAGTAACTGAGAGGTGGGAAGCTCAAAGCCAAA
GTCCTAATGTGCAGTCAGGTGAATTTATTGAAAAATTTGAGGCTCCTGGT
GGTGCAAATCAAAGAACTGCTCCTCAGGGATCCTAATTGTTTGTGTATTT
TAGGCggccgcatgaacgctctccaagaagatactccacccggaccatca
acggtgttcaggccaccaacaagttcacggccgttggaaacccctcactg
cagagagatccggattggtatcgctggaattacaatcactctatcgctgt
gtggctgcgcgaatgctcgcgctcccacgctaagatctgcaactgcggac
aattcagaaagcactggtttcaagaatgtgccggacttgaggaccgatca
acccaagcctccctcgaagaagcgatcctgcgacccctccgagtacaggg
taagcgagctaaaagaaagcttgattaccactactcccagccgaccccga
accgcaagaaggcgtataagactgtaagcggccgctgagggcagaggaag
tcttctaacatgcggtgacgtggaggagaatcccggcccttccggaATGG
AGAGCGACGAGAGCGGCCTGCCCGCCATGGAGATCGAGTGCCGCATCACC
GGCACCCTGAACGGCGTGGAGTTCGAGCTGGTGGGCGGCGGAGAGGGCAC
CCCCAAGCAGGGCCGCATGACCAACAAGATGAAGAGCACCAAAGGCGCCC
TGACCTTCAGCCCCTACCTGCTGAGCCACGTGATGGGCTACGGCTTCTAC
CACTTCGGCACCTACCCCAGCGGCTACGAGAACCCCTTCCTGCACGCCAT
CAACAACGGCGGCTACACCAACACCCGCATCGAGAAGTACGAGGACGGCG
GCGTGCTGCACGTGAGCTTCAGCTACCGCTACGAGGCCGGCCGCGTGATC
GGCGACTTCAAGGTGGTGGGCACCGGCTTCCCCGAGGACAGCGTGATCTT
CACCGACAAGATCATCCGCAGCAACGCCACCGTGGAGCACCTGCACCCCA
TGGGCGATAACGTGCTGGTGGGCAGCTTCGCCCGCACCTTCAGCCTGCGC
GACGGCGGCTACTACAGCTTCGTGGTGGACAGCCACATGCACTTCAAGAG
CGCCATCCACCCCAGCATCCTGCAGAACGGGGGCCCCATGTTCGCCTTCC
GCCGCGTGGAGGAGCTGCACAGCAACACCGAGCTGGGCATCGTGGAGTAC
CAGCACGCCTTCAAGACCCCCATCGCCTTCGCCAGATCCCGCGCTCAGTC
GTCCAATTCTGCCGTGGACGGCACCGCCGGACCCGGCTCCACCGGATCTC
GCTAGAGCTGAATCtaagtcgacaatcaacctctggattacaaaatttgt
gaaagattgactggtattcttaactatgttgctccttttacgctatgtgg
atacgctgctttaatgcctttgtatcatgctattgcttcccgtatggctt
tcattttctcctccttgtataaatcctggttgctgtctctttatgaggag
ttgtggcccgttgtcaggcaacgtggcgtggtgtgcactgtgtttgctga
cgcaacccccactggttggggcattgccaccacctgtcagctcctttccg
ggactttcgctttccccctccctattgccacggcggaactcatcgccgcc
tgccttgcccgctgctggacaggggctcggctgttgggcactgacaattc
cgtggtgttgtcggggaaatcatcgtcctttccttggctgctcgcctgtg
ttgccacctggattctgcgcgggacgtccttctgctacgtcccttcggcc
ctcaatccagcggaccttccttcccgcggcctgctgccggctctgcggcc
tcttccgcgtcttcgccttcgccctcagacgagtcggatctccctttggg
ccgcctccccgcccgggACACACCAAGGATAATTTCTCCCGATACACACC
AAGGATAATTTCTCCTCACACACACCAAGGATAATTTCTCCCGATACACA
CCAAGGATAATTTCTCCTCACAACTGGATGTCCCTGTATGATTCGATAAC
TGGATGTCCCTGTATGATTTCACAACTGGATGTCCCTGTATGATTCGATA
ACTGGATGTCCCTGTATGATTTCACAAAGAGGTTAACCATGTATTATCGA
TAAAGAGGTTAACCATGTATTATTCACAAAGAGGTTAACCATGTATTATC
GATAAAGAGGTTAACCATGTATTATTCACGGTTAGTGGACCGTGTTACAT
ACGATGGTTAGTGGACCGTGTTACATATCACGGTTAGTGGACCGTGTTAC
ATACGATGGTTAGTGGACCGTGTTACATATCACGAATTCACCAAGGGCAA
CCTCTCGATGAATTCACCAAGGGCAACCTCTTCACGAATTCACCAAGGGC
AACCTCTCGATGAATTCACCAAGGGCAACCTCTTCACAACTCCTGTATGA
AGCCGTTCCGATAACTCCTGTATGAAGCCGTTCTCACAACTCCTGTATGA
AGCCGTTCCGATAACTCCTGTATGAAGCCGTTCTCACATGGCAATATGTT
GGCATAGCACGATATGGCAATATGTTGGCATAGCATCACATGGCAATATG
TTGGCATAGCACGATATGGCAATATGTTGGCATAGCATCACGCACAGCAA
GTGTAGACAGGCACGATGCACAGCAAGTGTAGACAGGCATCACGCACAGC
AAGTGTAGACAGGCACGATGCACAGCAAGTGTAGACAGGCATCACAACAT
ACAAAGGGTATCCTCTCGATAACATACAAAGGGTATCCTCTTCACAACAT
ACAAAGGGTATCCTCTCGATAACATACAAAGGGTATCCTCTTCACCTCCC
TTCTTTCCTCCCGTCTTCGATCTCCCTTCTTTCCTCCCGTCTTTCACCTC
CCTTCTTTCCTCCCGTCTTCGATCTCCCTTCTTTCCTCCCGTCTTcccgg
gtacctttaagaccaatgacttacaaggcagctgtagatcttagccactt
tttaaaagaaaaggggggactggaagggctaattcactcccaacgaaaat
aagatctgctttttgcttgtactgggtctctctggttagaccagatctga
gcctgggagctctctggctaactagggaacccactgcttaagcctcaata
aagcttgccttgagtgcttcaagtagtgtgtgcccgtctgttgtgtgact
ctggtaactagagatccctcagacccttttagtcagtgtggaaaatctct
agcagtagtagttcatgtcatcttattattcagtatttataacttgcaaa
gaaatgaatatcagagagtgagaggaacttgtttattgcagcttataatg
gttacaaataaagcaatagcatcacaaatttcacaaataaagcatttttt
tcactgcattctagttgtggtttgtccaaactcatcaatgtatcttatca
tgtctggctctagctatcccgcccctaactccgcccagttccgcccattc
tccgccccatggctgactaattttttttatttatgcagaggccgaggccg
cctcggcctctgagctattccagaagtagtgaggaggcttttttggaggc
ctagacttttgcagagacggcccaaattcgtaatcatggtcatagctgtt
tcctgtgtgaaattgttatccgctcacaattccacacaacatacgagccg
gaagcataaagtgtaaagcctggggtgcctaatgagtgagctaactcaca
ttaattgcgttgcgctcactgcccgctttccagtcgggaaacctgtcgtg
ccagctgcattaatgaatcggccaacgcgcggggagaggcggtttgcgta
ttgggcgctcttccgcttcctcgctcactgactcgctgcgctcggtcgtt
cggctgcggcgagcggtatcagctcactcaaaggcggtaatacggttatc
cacagaatcaggggataacgcaggaaagaacatgtgagcaaaaggccagc
aaaaggccaggaaccgtaaaaaggccgcgttgctggcgtttttccatagg
ctccgcccccctgacgagcatcacaaaaatcgacgctcaagtcagaggtg
gcgaaacccgacaggactataaagataccaggcgtttccccctggaagct
ccctcgtgcgctctcctgttccgaccctgccgcttaccggatacctgtcc
gcctttctcccttcgggaagcgtggcgctttctcatagctcacgctgtag
gtatctcagttcggtgtaggtcgttcgctccaagctgggctgtgtgcacg
aaccccccgttcagcccgaccgctgcgccttatccggtaactatcgtctt
gagtccaacccggtaagacacgacttatcgccactggcagcagccactgg
taacaggattagcagagcgaggtatgtaggcggtgctacagagttcttga
agtggtggcctaactacggctacactagaaggacagtatttggtatctgc
gctctgctgaagccagttaccttcggaaaaagagttggtagctcttgatc
cggcaaacaaaccaccgctggtagcggtggtttttttgtttgcaagcagc
agattacgcgcagaaaaaaaggatctcaagaagatcctttgatcttttct
acggggtctgacgctcagtggaacgaaaactcacgttaagggattttggt
catgagattatcaaaaaggatcttcacctagatccttttaaattaaaaat
gaagttttaaatcaatctaaagtatatatgagtaaacttggtctgacagt
taccaatgcttaatcagtgaggcacctatctcagcgatctgtctatttcg
ttcatccatagttgcctgactccccgtcgtgtagataactacgatacggg
agggcttaccatctggccccagtgctgcaatgataccgcgagacccacgc
tcaccggctccagatttatcagcaataaaccagccagccggaagggccga
gcgcagaagtggtcctgcaactttatccgcctccatccagtctattaatt
gttgccgggaagctagagtaagtagttcgccagttaatagtttgcgcaac
gttgttgccattgctacaggcatcgtggtgtcacgctcgtcgtttggtat
ggcttcattcagctccggttcccaacgatcaaggcgagttacatgatccc
ccatgttgtgcaaaaaagcggttagctccttcggtcctccgatcgttgtc
agaagtaagttggccgcagtgttatcactcatggttatggcagcactgca
taattctcttactgtcatgccatccgtaagatgcttttctgtgactggtg
agtactcaaccaagtcattctgagaatagtgtatgcggcgaccgagttgc
tcttgcccggcgtcaatacgggataataccgcgccacatagcagaacttt
aaaagtgctcatcattggaaaacgttcttcggggcgaaaactctcaagga
tcttaccgctgttgagatccagttcgatgtaacccactcgtgcacccaac
tgatcttcagcatcttttactttcaccagcgtttctgggtgagcaaaaac
aggaaggcaaaatgccgcaaaaaagggaataagggcgacacggaaatgtt
gaatactcatactcttcctttttcaatattattgaagcatttatcagggt
tattgtctcatgagcggatacatatttgaatgtatttagaaaaataaaca
aataggggttccgcgcacatttccccgaaaagtgccacctgacgtctaag
aaaccattattatcatgacattaacctataaaaataggcgtatcacgagg
ccctttcgtctcgcgcgtttcggtgatgacggtgaaaacctctgacacat
gcagctcccggagacggtcacagcttgtctgtaagcggatgccgggagca
gacaagcccgtcagggcgcgtcagcgggtgttggcgggtgtcggggctgg
cttaactatgcggcatcagagcagattgtactgagagtgcaccatatgcg
gtgtgaaataccgcacagatgcgtaaggagaaaataccgcatcaggcgcc
attcgccattcaggctgcgcaactgttgggaagggcgatcggtgcgggcc
tcttcgctattacgccagctggcgaaagggggatgtgctgcaaggcgatt
aagttgggtaacgccagggttttcccagtcacgacgttgtaaaacgacgg
ccagtgccaagctg
[SEQ ID No: 80]

Preferably, the genetic construct comprises a nucleic acid sequence substantially as set out in SEQ ID No: 80, or a fragment or variant thereof.

Another embodiment of the genetic construct is shown in FIG. 8F and may comprise an E4orf4 encoding sequence and be represented by SEQ ID No: 24, as follows:

acgcgtgtagtcttatgcaatactcttgtagtcttgcaacatggtaacga
tgagttagcaacatgccttacaaggagagaaaaagcaccgtgcatgccga
ttggtggaagtaaggtggtacgatcgtgccttattaggaaggcaacagac
gggtctgacatggattggacgaaccactgaattgccgcattgcagagata
ttgtatttaagtgcctagctcgatacaataaacgggtctctctggttaga
ccagatctgagcctgggagctctctggctaactagggaacccactgctta
agcctcaataaagcttgccttgagtgcttcaagtagtgtgtgcccgtctg
ttgtgtgactctggtaactagagatccctcagacccttttagtcagtgtg
gaaaatctctagcagtggcgcccgaacagggacctgaaagcgaaagggaa
accagagctctctcgacgcaggactcggcttgctgaagcgcgcacggcaa
gaggcgaggggcggcgactggtgagtacgccaaaaattttgactagcgga
ggctagaaggagagagatgggtgcgagagcgtcagtattaagcgggggag
aattagatcgcgatgggaaaaaattcggttaaggccagggggaaagaaaa
aatataaattaaaacatatagtatgggcaagcagggagctagaacgattc
gcagttaatcctggcctgttagaaacatcagaaggctgtagacaaatact
gggacagctacaaccatcccttcagacaggatcagaagaacttagatcat
tatataatacagtagcaaccctctattgtgtgcatcaaaggatagagata
aaagacaccaaggaagctttagacaagatagaggaagagcaaaacaaaag
taagaccaccgcacagcaagcggccactgatcttcagacctggaggagga
gatatgagggacaattggagaagtgaattatataaatataaagtagtaaa
aattgaaccattaggagtagcacccaccaaggcaaagagaagagtggtgc
agagagaaaaaagagcagtgggaataggagctttgttccttgggttcttg
ggagcagcaggaagcactatgggcgcagcctcaatgacgctgacggtaca
ggccagacaattattgtctggtatagtgcagcagcagaacaatttgctga
gggctattgaggcgcaacagcatctgttgcaactcacagtctggggcatc
aagcagctccaggcaagaatcctggctgtggaaagatacctaaaggatca
acagctcctggggatttggggttgctctggaaaactcatttgcaccactg
ctgtgccttggaatgctagttggagtaataaatctctggaacagattTgg
aatcacacgacctggatggagtgggacagagaaattaacaattacacaag
cttaatacactccttaattgaagaatcgcaaaaccagcaagaaaagaatg
aacaagaattattggaattagataaatgggcaagtttgtggaattggttt
aacataacaaattggctgtggtatataaaattattcataatgatagtagg
aggcttggtaggtttaagaatagtttttgctgtactttctatagtgaata
gagttaggcagggatattcaccattatcgtttcagacccacctcccaacc
ccgaggggacccgacaggcccgaaggaatagaagaagaaggtggagagag
agacagagacagatccattcgattagtgaacggatctcgacggtATCGGT
taacttttaaaagaaaaggggggattggggggtacagtgcaggggaaaga
atagtagacataatagcaacagacatacaaactaaagaattacaaaaaca
aattacaaaaattcaaaattttatGTCGAGccatagagcccaccgcatcc
ccagcatgcctgctattgtcttcccaatcctcccccttgctgtcctgccc
caccccaccccccagaatagaatgacacctactcagacaatgcgatgcaa
tttcctcattttattaggaaaggacagtgggagtggcaccttccagggtc
aaggaaggcacgggggaggggcaaacaacagatggctggcaactagaagg
cacagtcgCtcgagAGTAAAACACTACAACGTCGATAGCAGTAAAACACT
ACAACGTCGACACTAGTAAAACACTACAACGTCGATAGCAGTAAAACACT
ACAACGTCGACACTAGTGTCACTTGGCCAGAGAAATAGCAGTGTCACTTG
GCCAGAGAAACACTAGTGTCACTTGGCCAGAGAAATAGCAGTGTCACTTG
GCCAGAGAAActcgagTCAAACCTTCCTCTTCTTCTTAGGAGGCCTCAGG
CTGCTCTGCCCGCTTTCCAGTCGGGAAACCTGTCGTGCCAGCTGCATTAA
TGAATCGGCCAACGCGCGGGGAGAGGCGGTTTGCGTATTGGGCGCCAGGG
TGGTTTTTCTTTTCACCAGTGAGACGGGCAACAGCTGATTGCCCTTCACC
GCCTGGCCCTGAGAGAGTTGCAGCAAGCGGTCCACGCTGGTTTGCCCCAG
CAGGCGAAAATCCTGTTTGATGGTGGTTAACGGCGGGATATAACATGAGC
TGTCTTCGGTATCGTCGTATCCCACTACCGAGATATCCGCACCAACGCGC
AGCCCGGACTCGGTAATGGCGCGCATTGCGCCCAGCGCCATCTGATCGTT
GGCAACCAGCATCGCAGTGGGAACGATGCCCTCATTCAGCATTTGCATGG
TTTGTTGAAAACCGGACATGGCACTCCAGTCGCCTTCCCGTTCCGCTATC
GGCTGAATTTGATTGCGAGTGAGATATTTATGCCAGCCAGCCAGACGCAG
ACGCGCCGAGACAGAACTTAATGGGCCCGCTAACAGCGCGATTTGCTGGT
GACCCAATGCGACCAGATGCTCCACGCCCAGTCGCGTACCGTCTTCATGG
GAGAAAATAATACTGTTGATGGGTGTCTGGTCAGAGACATCAAGAAATAA
CGCCGGAACATTAGTGCAGGCAGCTTCCACAGCAATGGCATCCTGGTCAT
CCAGCGGATAGTTAATGATCAGCCCACTGACGCGTTGCGCGAGAAGATTG
TGCACCGCCGCTTTACAGGCTTCGACGCCGCTTCGTTCTACCATCGACAC
CACCACGCTGGCACCCAGTTGATCGGCGCGAGATTTAATCGCCGCGACAA
TTTGCGACGGCGCGTGCAGGGCCAGACTGGAGGTGGCAACGCCAATCAGC
AACGACTGTTTGCCCGCCAGTTGTTGTGCCACGCGGTTGGGAATGTAATT
CAGCTCCGCCATCGCCGCTTCCACTTTTTCCCGCGTTTTCGCAGAAACGT
GGCTGGCCTGGTTCACCACGCGGGAAACGGTCTGATAAGAGACACCGGCA
TACTCTGCGACATCGTATAACGTTACTGGTTTCATGGTGGGAGGGTACcT
AGGTAAGCTTGGGCTGCAGGTCGAAAGGCCCGGAGATGAGGAAGAGGAGA
ACAGCGCGGCAGACGTGCGCTTTTGAAGCGTGCAGAATGCCGGGCCTCCG
GAGGACCTTCGGGCGCCCGCCCCGCCCCTGAGCCCGCCCCTGAGCCCGCC
CCCGGACCCACCCCTTCCCAGCCTCTGAGCCCAGAAAGCGAAGGAGCAAA
GCTGCTATTGGCCGCTGCCCCAAAGGCCTACCCGCTTCCATTGCTCAGCG
GTGCTGTCCATCTGCACGAGACTAGTGAGACGTGCTACTTCCATTTGTCA
CGTCCTGCACGACGCGAGCTGCGGGGCGGGGGGGAACTTCCTGACTAGGG
GAGGAGTAGAAGGTGGCGCGAAGGGGCCACCAAAGAACGGAGCCGGTTGG
CGCCTACCGGTGGATGTGGAATGTGTGCGAGGCCAGAGGCCACTTGTGTA
GCGCCAAGTGCCCAGCGGGGCTGCTAAAGCGCATGCTCCAGACTGCCTTG
GGAAAAGCGCCTCCCCTACCCGGTAGAAGCTAGCGGCTCCGGTGCCCGTC
AGTGGGCAGAGCGCACATCGCCCACAGTCCCCGAGAAGTTGTGGGGAGGG
GTCGGCAATTGAACCGGTGCCTAGAGAAGGTGGCGCGGGGTAAACTGGGA
AAGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTTT
GCCGCCAGAACACAGGTAAGTGCCGTGTGTGGTTCCCGCGGGCCTGGCCT
CTTTACGGGTTATGGCCCTTGCGTGCCTTGAATTACTTCCACCTGGCTGC
AGTACGTGATTCTTGATCCCGAGCTTCGGGTTGGAAGTGGGTGGGAGAGT
TCGAGGCCTTGCGCTTAAGGAGCCCCTTCGCCTCGTGCTTGAGTTGAGGC
CTGGCCTGGGCGCTGGGGCCGCCGCGTGCGAATCTGGTGGCACCTTCGCG
CCTGTCTCGCTGCTTTCGATAAGTCTCTAGCCATTTAAAATTTTTGATGA
CCTGCTGCGACGCTTTTTTTCTGGCAAGATAGTCTTGTAAATGCGGGCCA
AGATCTGCACACTGGTATTTCGGTTTTTGGGGCCGCGGGCGGCGACGGGG
CCCGTGCGTCCCAGCGCACATGTTCGGCGAGGCGGGGCCTGCGAGCGCGG
CCACCGAGAATCGGACGGGGGTAGTCTCAAGCTGGCCGGCCTGCTCTGGT
GCCTGGCCTCGCGCCGCCGTGTATCGCCCCGCCCTGGGCGGCAAGGCTGG
CCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGG
TGAGTCACCCACACAAAGGAAAAGGGCCTTTCCGTCCTCAGCCGTCGCTT
CATGTGACTCCACGGAGTACCGGGCGCCGTCCAGGCACCTCGATTAGTTC
TCGACCTTTTGGAGTACGTCGTCTTTAGGTTGGGGGGAGGGGTTTTATGC
GATGGAGTTTCCCCACACTGAGTGGGTGGAGACTGAAGTTAGGCCAGCTT
GGCACTTGATGTAATTCTCCTTGGAATTTGCCCTTTTTGAGTTTGGATCT
TGGTTCATTCTCAAGCCTCAGACAGTGGTTCAAAGTTTTTTTCTTCCATT
TCAGGTGTCGTGAtctagagctagcgAATTCGTAAATATAAAATTTACTA
GGTTGTGGAATTGTGAGCGCTCACAATTCCACAGTCGACCCTAGGTTGTG
GAATTGTGAGCGCTCACAATTCCACAGTCGACCCTAGGTTGTGGAATTGT
GAGCGCTCACAATTCCACAGTCGACCCTAGTGTATAATGTGTTAAACTAC
GGATCCGTCTCCCATTAGGCCTACAATGGTGAGACAAGTAGCCAACAGGG
AAGGGTTGCAAATATCATTTGGGCACACCTATGATAATATTGATGAAGCA
GACAGTATTCAGCAAGTAACTGAGAGGTGGGAAGCTCAAAGCCAAAGTCC
TAATGTGCAGTCAGGTGAATTTATTGAAAAATTTGAGGCTCCTGGTGGTG
CAAATCAAAGAACTGCTCCTCAGGGATCCTAATTGTTTGTGTATTTTAGG
Cggccgcatggttcttccagctcttcccgctcctcccgtgtgtgactcgc
agaacgaatgtgtaggttggctgggtgtggcttattctgcggtggtggat
gttatcagggcagcggcgcatgaaggagtttacatagaacccgaagccag
ggggcgcctggatgctttgagagagtggatatactacaactactacacag
agcgagctaagcgacgagaccggagacgcagatctgtttgtcacgcccgc
acctggttttgcttcaggaaatatgactacgtccggcgttccatttggca
tgacactacgaccaacacgatctcggttgtctcggcgcactccgtacagt
agGCGGCCGCtgagggcagaggaagtcttctaacatgcggtgacgtggag
gagaatcccggcccttccggaATGGAGAGCGACGAGAGCGGCCTGCCCGC
CATGGAGATCGAGTGCCGCATCACCGGCACCCTGAACGGCGTGGAGTTCG
AGCTGGTGGGCGGCGGAGAGGGCACCCCCAAGCAGGGCCGCATGACCAAC
AAGATGAAGAGCACCAAAGGCGCCCTGACCTTCAGCCCCTACCTGCTGAG
CCACGTGATGGGCTACGGCTTCTACCACTTCGGCACCTACCCCAGCGGCT
ACGAGAACCCCTTCCTGCACGCCATCAACAACGGCGGCTACACCAACACC
CGCATCGAGAAGTACGAGGACGGCGGCGTGCTGCACGTGAGCTTCAGCTA
CCGCTACGAGGCCGGCCGCGTGATCGGCGACTTCAAGGTGGTGGGCACCG
GCTTCCCCGAGGACAGCGTGATCTTCACCGACAAGATCATCCGCAGCAAC
GCCACCGTGGAGCACCTGCACCCCATGGGCGATAACGTGCTGGTGGGCAG
CTTCGCCCGCACCTTCAGCCTGCGCGACGGCGGCTACTACAGCTTCGTGG
TGGACAGCCACATGCACTTCAAGAGCGCCATCCACCCCAGCATCCTGCAG
AACGGGGGCCCCATGTTCGCCTTCCGCCGCGTGGAGGAGCTGCACAGCAA
CACCGAGCTGGGCATCGTGGAGTACCAGCACGCCTTCAAGACCCCCATCG
CCTTCGCCAGATCCCGCGCTCAGTCGTCCAATTCTGCCGTGGACGGCACC
GCCGGACCCGGCTCCACCGGATCTCGCTAGAGCTGAATCtaagtcgacaa
tcaacctctggattacaaaatttgtgaaagattgactggtattcttaact
atgttgctccttttacgctatgtggatacgctgctttaatgcctttgtat
catgctattgcttcccgtatggctttcattttctcctccttgtataaatc
ctggttgctgtctctttatgaggagttgtggcccgttgtcaggcaacgtg
gcgtggtgtgcactgtgtttgctgacgcaacccccactggttggggcatt
gccaccacctgtcagctcctttccgggactttcgctttccccctccctat
tgccacggcggaactcatcgccgcctgccttgcccgctgctggacagggg
ctcggctgttgggcactgacaattccgtggtgttgtcggggaaatcatcg
tcctttccttggctgctcgcctgtgttgccacctggattctgcgcgggac
gtccttctgctacgtcccttcggccctcaatccagcggaccttccttccc
gcggcctgctgccggctctgcggcctcttccgcgtcttcgccttcgccct
cagacgagtcggatctccctttgggccgcctccccgcccgggTAACCGAT
TTCAGATGGTGCTACGATTAACCGATTTCAGATGGTGCTATCACTAACCG
ATTTCAGATGGTGCTACGATTAACCGATTTCAGATGGTGCTATCACGGGA
GTGAAGACACGGAGCCAGACGATGGGAGTGAAGACACGGAGCCAGATCAC
GGGAGTGAAGACACGGAGCCAGACGATGGGAGTGAAGACACGGAGCCAGA
cccgggtacctttaagaccaatgacttacaaggcagctgtagatcttagc
cactttttaaaagaaaaggggggactggaagggctaattcactcccaacg
aaaataagatctgctttttgcttgtactgggtctctctggttagaccaga
tctgagcctgggagctctctggctaactagggaacccactgcttaagcct
caataaagcttgccttgagtgcttcaagtagtgtgtgcccgtctgttgtg
tgactctggtaactagagatccctcagacccttttagtcagtgtggaaaa
tctctagcagtagtagttcatgtcatcttattattcagtatttataactt
gcaaagaaatgaatatcagagagtgagaggaacttgtttattgcagctta
taatggttacaaataaagcaatagcatcacaaatttcacaaataaagcat
ttttttcactgcattctagttgtggtttgtccaaactcatcaatgtatct
tatcatgtctggctctagctatcccgcccctaactccgcccagttccgcc
cattctccgccccatggctgactaattttttttatttatgcagaggccga
ggccgcctcggcctctgagctattccagaagtagtgaggaggcttttttg
gaggcctagacttttgcagagacggcccaaattcgtaatcatggtcatag
ctgtttcctgtgtgaaattgttatccgctcacaattccacacaacatacg
agccggaagcataaagtgtaaagcctggggtgcctaatgagtgagctaac
tcacattaattgcgttgcgctcactgcccgctttccagtcgggaaacctg
tcgtgccagctgcattaatgaatcggccaacgcgcggggagaggcggttt
gcgtattgggcgctcttccgcttcctcgctcactgactcgctgcgctcgg
tcgttcggctgcggcgagcggtatcagctcactcaaaggcggtaatacgg
ttatccacagaatcaggggataacgcaggaaagaacatgtgagcaaaagg
ccagcaaaaggccaggaaccgtaaaaaggccgcgttgctggcgtttttcc
ataggctccgcccccctgacgagcatcacaaaaatcgacgctcaagtcag
aggtggcgaaacccgacaggactataaagataccaggcgtttccccctgg
aagctccctcgtgcgctctcctgttccgaccctgccgcttaccggatacc
tgtccgcctttctcccttcgggaagcgtggcgctttctcatagctcacgc
tgtaggtatctcagttcggtgtaggtcgttcgctccaagctgggctgtgt
gcacgaaccccccgttcagcccgaccgctgcgccttatccggtaactatc
gtcttgagtccaacccggtaagacacgacttatcgccactggcagcagcc
actggtaacaggattagcagagcgaggtatgtaggcggtgctacagagtt
cttgaagtggtggcctaactacggctacactagaaggacagtatttggta
tctgcgctctgctgaagccagttaccttcggaaaaagagttggtagctct
tgatccggcaaacaaaccaccgctggtagcggtggtttttttgtttgcaa
gcagcagattacgcgcagaaaaaaaggatctcaagaagatcctttgatct
tttctacggggtctgacgctcagtggaacgaaaactcacgttaagggatt
ttggtcatgagattatcaaaaaggatcttcacctagatccttttaaatta
aaaatgaagttttaaatcaatctaaagtatatatgagtaaacttggtctg
acagttaccaatgcttaatcagtgaggcacctatctcagcgatctgtcta
tttcgttccatccatagttgcctgactccccgtcgtgtagataactacga
tacgggagggcttaccatctggccccagtgctgcaatgataccgcgagac
ccacgctcaccggctccagatttatcagcaataaaccagccagccggaag
ggccgagcgcagaagtggtcctgcaactttatccgcctccatccagtcta
ttaattgttgccgggaagctagagtaagtagttcgccagttaatagtttg
cgcaacgttgttgccattgctacaggcatcgtggtgtcacgctcgtcgtt
tggtatggcttcattcagctccggttcccaacgatcaaggcgagttacat
gatcccccatgttgtgcaaaaaagcggttagctccttcggtcctccgatc
gttgtcagaagtaagttggccgcagtgttatcactcatggttatggcagc
actgcataattctcttactgtcatgccatccgtaagatgcttttctgtga
ctggtgagtactcaaccaagtcattctgagaatagtgtatgcggcgaccg
agttgctcttgcccggcgtcaatacgggataataccgcgccacatagcag
aactttaaaagtgctcatcattggaaaacgttcttcggggcgaaaactct
caaggatcttaccgctgttgagatccagttcgatgtaacccactcgtgca
cccaactgatcttcagcatcttttactttcaccagcgtttctgggtgagc
aaaaacaggaaggcaaaatgccgcaaaaaagggaataagggcgacacgga
aatgttgaatactcatactcttcctttttcaatattattgaagcatttat
cagggttattgtctcatgagcggatacatatttgaatgtatttagaaaaa
taaacaaataggggttccgcgcacatttccccgaaaagtgccacctgacg
tctaagaaaccattattatcatgacattaacctataaaaataggcgtatc
acgaggccctttcgtctcgcgcgtttcggtgatgacggtgaaaacctctg
acacatgcagctcccggagacggtcacagcttgtctgtaagcggatgccg
ggagcagacaagcccgtcagggcgcgtcagcgggtgttggcgggtgtcgg
ggctggcttaactatgcggcatcagagcagattgtactgagagtgcacca
tatgcggtgtgaaataccgcacagatgcgtaaggagaaaataccgcatca
ggcgccattcgccattcaggctgcgcaactgttgggaagggcgatcggtg
cgggcctcttcgctattacgccagctggcgaaagggggatgtgctgcaag
gcgattaagttgggtaacgccagggttttcccagtcacgacgttgtaaaa
cgacggccagtgccaagctg
[SEQ ID No
: 24]

Preferably, the genetic construct comprises a nucleic acid sequence substantially as set out in SEQ ID No: 24, or a fragment or variant thereof.

Another embodiment of the genetic construct comprising an E4orf4 encoding sequence is shown in FIG. 8K and is represented by SEQ ID No: 81, as follows:

acgcgtgtagtcttatgcaatactcttgtagtcttgcaacatggtaacga
tgagttagcaacatgccttacaaggagagaaaaagcaccgtgcatgccga
ttggtggaagtaaggtggtacgatcgtgccttattaggaaggcaacagac
gggtctgacatggattggacgaaccactgaattgccgcattgcagagata
ttgtatttaagtgcctagctcgatacaataaacgggtctctctggttaga
ccagatctgagcctgggagctctctggctaactagggaacccactgctta
agcctcaataaagcttgccttgagtgcttcaagtagtgtgtgcccgtctg
ttgtgtgactctggtaactagagatccctcagacccttttagtcagtgtg
gaaaatctctagcagtggcgcccgaacagggacctgaaagcgaaagggaa
accagagctctctcgacgcaggactcggcttgctgaagcgcgcacggcaa
gaggcgaggggcggcgactggtgagtacgccaaaaattttgactagcgga
ggctagaaggagagagatgggtgcgagagcgtcagtattaagcgggggag
aattagatcgcgatgggaaaaaattcggttaaggccagggggaaagaaaa
aatataaattaaaacatatagtatgggcaagcagggagctagaacgattc
gcagttaatcctggcctgttagaaacatcagaaggctgtagacaaatact
gggacagctacaaccatcccttcagacaggatcagaagaacttagatcat
tatataatacagtagcaaccctctattgtgtgcatcaaaggatagagata
aaagacaccaaggaagctttagacaagatagaggaagagcaaaacaaaag
taagaccaccgcacagcaagcggccactgatcttcagacctggaggagga
gatatgagggacaattggagaagtgaattatataaatataaagtagtaaa
aattgaaccattaggagtagcacccaccaaggcaaagagaagagtggtgc
agagagaaaaaagagcagtgggaataggagctttgttccttgggttcttg
ggagcagcaggaagcactatgggcgcagcctcaatgacgctgacggtaca
ggccagacaattattgtctggtatagtgcagcagcagaacaatttgctga
gggctattgaggcgcaacagcatctgttgcaactcacagtctggggcatc
aagcagctccaggcaagaatcctggctgtggaaagatacctaaaggatca
acagctcctggggatttggggttgctctggaaaactcatttgcaccactg
ctgtgccttggaatgctagttggagtaataaatctctggaacagattTgg
aatcacacgacctggatggagtgggacagagaaattaacaattacacaag
cttaatacactccttaattgaagaatcgcaaaaccagcaagaaaagaatg
aacaagaattattggaattagataaatgggcaagtttgtggaattggttt
aacataacaaattggctgtggtatataaaattattcataatgatagtagg
aggcttggtaggtttaagaatagtttttgctgtactttctatagtgaata
gagttaggcagggatattcaccattatcgtttcagacccacctcccaacc
ccgaggggacccgacaggcccgaaggaatagaagaagaaggtggagagag
agacagagacagatccattcgattagtgaacggatctcgacggtATCGGT
taacttttaaaagaaaaggggggattggggggtacagtgcaggggaaaga
atagtagacataatagcaacagacatacaaactaaagaattacaaaaaca
aattacaaaaattcaaaattttatGTCGAGccatagagcccaccgcatcc
ccagcatgcctgctattgtcttcccaatcctcccccttgctgtcctgccc
caccccaccccccagaatagaatgacacctactcagacaatgcgatgcaa
tttcctcattttattaggaaaggacagtgggagtggcaccttccagggtc
aaggaaggcacgggggaggggcaaacaacagatggctggcaactagaagg
cacagtcgCtcgagGGGCCTGTCCGCAAGCACGCTGCATAGCGGGCCTGT
CCGCAAGCACGCTGCACACTGGGCCTGTCCGCAAGCACGCTGCATAGCGG
GCCTGTCCGCAAGCACGCTGCACACTCCCACCCCTAAACAACGTAATGTA
GCCCCACCCCTAAACAACGTAATGCACTCCCACCCCTAAACAACGTAATG
TAGCCCCACCCCTAAACAACGTAATGCACTTGACATCATACCCGTGAAGG
TCTAGCTGACATCATACCCGTGAAGGTCCACTTGACATCATACCCGTGAA
GGTCTAGCTGACATCATACCCGTGAAGGTCCACTACTCCGCCCCCCCGCT
CGTAGCACTCCGCCCCCCCGCTCGCACTACTCCGCCCCCCCGCTCGTAGC
ACTCCGCCCCCCCGCTCGCACTGACGTTACATTCGTGAAGAATGTAGCGA
CGTTACATTCGTGAAGAATGCACTGACGTTACATTCGTGAAGAATGTAGC
GACGTTACATTCGTGAAGAATGCACTGCCCCAGCCGCCGCTGCACTAGCG
CCCCAGCCGCCGCTGCACCACTGCCCCAGCCGCCGCTGCACTAGCGCCCC
AGCCGCCGCTGCACctcgagTCAAACCTTCCTCTTCTTCTTAGGAGGCCT
CAGGCTGCTCTGCCCGCTTTCCAGTCGGGAAACCTGTCGTGCCAGCTGCA
TTAATGAATCGGCCAACGCGCGGGGAGAGGCGGTTTGCGTATTGGGCGCC
AGGGTGGTTTTTCTTTTCACCAGTGAGACGGGCAACAGCTGATTGCCCTT
CACCGCCTGGCCCTGAGAGAGTTGCAGCAAGCGGTCCACGCTGGTTTGCC
CCAGCAGGCGAAAATCCTGTTTGATGGTGGTTAACGGCGGGATATAACAT
GAGCTGTCTTCGGTATCGTCGTATCCCACTACCGAGATATCCGCACCAAC
GCGCAGCCCGGACTCGGTAATGGCGCGCATTGCGCCCAGCGCCATCTGAT
CGTTGGCAACCAGCATCGCAGTGGGAACGATGCCCTCATTCAGCATTTGC
ATGGTTTGTTGAAAACCGGACATGGCACTCCAGTCGCCTTCCCGTTCCGC
TATCGGCTGAATTTGATTGCGAGTGAGATATTTATGCCAGCCAGCCAGAC
GCAGACGCGCCGAGACAGAACTTAATGGGCCCGCTAACAGCGCGATTTGC
TGGTGACCCAATGCGACCAGATGCTCCACGCCCAGTCGCGTACCGTCTTC
ATGGGAGAAAATAATACTGTTGATGGGTGTCTGGTCAGAGACATCAAGAA
ATAACGCCGGAACATTAGTGCAGGCAGCTTCCACAGCAATGGCATCCTGG
TCATCCAGCGGATAGTTAATGATCAGCCCACTGACGCGTTGCGCGAGAAG
ATTGTGCACCGCCGCTTTACAGGCTTCGACGCCGCTTCGTTCTACCATCG
ACACCACCACGCTGGCACCCAGTTGATCGGCGCGAGATTTAATCGCCGCG
ACAATTTGCGACGGCGCGTGCAGGGCCAGACTGGAGGTGGCAACGCCAAT
CAGCAACGACTGTTTGCCCGCCAGTTGTTGTGCCACGCGGTTGGGAATGT
AATTCAGCTCCGCCATCGCCGCTTCCACTTTTTCCCGCGTTTTCGCAGAA
ACGTGGCTGGCCTGGTTCACCACGCGGGAAACGGTCTGATAAGAGACACC
GGCATACTCTGCGACATCGTATAACGTTACTGGTTTCATGGTGGGAGGGT
ACcTAGGTAAGCTTGGGCTGCAGGTCGAAAGGCCCGGAGATGAGGAAGAG
GAGAACAGCGCGGCAGACGTGCGCTTTTGAAGCGTGCAGAATGCCGGGCC
TCCGGAGGACCTTCGGGCGCCCGCCCCGCCCCTGAGCCCGCCCCTGAGCC
CGCCCCCGGACCCACCCCTTCCCAGCCTCTGAGCCCAGAAAGCGAAGGAG
CAAAGCTGCTATTGGCCGCTGCCCCAAAGGCCTACCCGCTTCCATTGCTC
AGCGGTGCTGTCCATCTGCACGAGACTAGTGAGACGTGCTACTTCCATTT
GTCACGTCCTGCACGACGCGAGCTGCGGGGCGGGGGGGAACTTCCTGACT
AGGGGAGGAGTAGAAGGTGGCGCGAAGGGGCCACCAAAGAACGGAGCCGG
TTGGCGCCTACCGGTGGATGTGGAATGTGTGCGAGGCCAGAGGCCACTTG
TGTAGCGCCAAGTGCCCAGCGGGGCTGCTAAAGCGCATGCTCCAGACTGC
CTTGGGAAAAGCGCCTCCCCTACCCGGTAGAAGCTAGCGGCTCCGGTGCC
CGTCAGTGGGCAGAGCGCACATCGCCCACAGTCCCCGAGAAGTTGTGGGG
AGGGGTCGGCAATTGAACCGGTGCCTAGAGAAGGTGGCGCGGGGTAAACT
GGGAAAGTGATGTCGTGTACTGGCTCCGCCTTTTTCCCGAGGGTGGGGGA
GAACCGTATATAAGTGCAGTAGTCGCCGTGAACGTTCTTTTTCGCAACGG
GTTTGCCGCCAGAACACAGGTAAGTGCCGTGTGTGGTTCCCGCGGGCCTG
GCCTCTTTACGGGTTATGGCCCTTGCGTGCCTTGAATTACTTCCACCTGG
CTGCAGTACGTGATTCTTGATCCCGAGCTTCGGGTTGGAAGTGGGTGGGA
GAGTTCGAGGCCTTGCGCTTAAGGAGCCCCTTCGCCTCGTGCTTGAGTTG
AGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGG
GCCAAGATCTGCACACTGGTATTTCGGTTTTTGGGGCCGCGGGCGGCGAC
GGGGCCCGTGCGTCCCAGCGCACATGTTCGGCGAGGCGGGGCCTGCGAGC
GCGGCCACCGAGAATCGGACGGGGGTAGTCTCAAGCTGGCCGGCCTGCTC
TGGTGCCTGGCCTCGCGCCGCCGTGTATCGCCCCGCCCTGGGCGGCAAGG
CTGGCCCGGTCGGCACCAGTTGCGTGAGCGGAAAGATGGCCGCTTCCCGG
CCCTGCTGCAGGGAGCTCAAAATGGAGGACGCGGCGCTCGGGAGAGCGGG
CGGGTGAGTCACCCACACAAAGGAAAAGGGCCTTTCCGTCCTCAGCCGTC
GCTTCATGTGACTCCACGGAGTACCGGGCGCCGTCCAGGCACCTCGATTA
GTTCTCGACCTTTTGGAGTACGTCGTCTTTAGGTTGGGGGGAGGGGTTTT
ATGCGATGGAGTTTCCCCACACTGAGTGGGTGGAGACTGAAGTTAGGCCA
GCTTGGCACTTGATGTAATTCTCCTTGGAATTTGCCCTTTTTGAGTTTGG
ATCTTGGTTCATTCTCAAGCCTCAGACAGTGGTTCAAAGTTTTTTTCTTC
CATTTCAGGTGTCGTGAtctagagctagcgAATTCGTAAATATAAAATTT
ACTAGGTTGTGGAATTGTGAGCGCTCACAATTCCACAGTCGACCCTAGGT
TGTGGAATTGTGAGCGCTCACAATTCCACAGTCGACCCTAGGTTGTGGAA
TTGTGAGCGCTCACAATTCCACAGTCGACCCTAGTGTATAATGTGTTAAA
CTACGGATCCGTCTCCCATTAGGCCTACAATGGTGAGACAAGTAGCCAAC
AGGGAAGGGTTGCAAATATCATTTGGGCACACCTATGATAATATTGATGA
AGCAGACAGTATTCAGCAAGTAACTGAGAGGTGGGAAGCTCAAAGCCAAA
GTCCTAATGTGCAGTCAGGTGAATTTATTGAAAAATTTGAGGCTCCTGGT
GGTGCAAATCAAAGAACTGCTCCTCAGGGATCCTAATTGTTTGTGTATTT
TAGGCggccgcatggttcttccagctcttcccgctcctcccgtgtgtgac
tcgcagaacgaatgtgtaggttggctgggtgtggcttattctgcggtggt
ggatgttatcagggcagcggcgcatgaaggagtttacatagaacccgaag
ccagggggcgcctggatgctttgagagagtggatatactacaactactac
acagagcgagctaagcgacgagaccggagacgcagatctgtttgtcacgc
ccgcacctggttttgcttcaggaaatatgactacgtccggcgttccattt
ggcatgacactacgaccaacacgatctcggttgtctcggcgcactccgta
cagtaggcggccgctgagggcagaggaagtcttctaacatgcggtgacgt
ggaggagaatcccggcccttccggaATGGAGAGCGACGAGAGCGGCCTGC
CCGCCATGGAGATCGAGTGCCGCATCACCGGCACCCTGAACGGCGTGGAG
TTCGAGCTGGTGGGCGGCGGAGAGGGCACCCCCAAGCAGGGCCGCATGAC
CAACAAGATGAAGAGCACCAAAGGCGCCCTGACCTTCAGCCCCTACCTGC
TGAGCCACGTGATGGGCTACGGCTTCTACCACTTCGGCACCTACCCCAGC
GGCTACGAGAACCCCTTCCTGCACGCCATCAACAACGGCGGCTACACCAA
CACCCGCATCGAGAAGTACGAGGACGGCGGCGTGCTGCACGTGAGCTTCA
GCTACCGCTACGAGGCCGGCCGCGTGATCGGCGACTTCAAGGTGGTGGGC
ACCGGCTTCCCCGAGGACAGCGTGATCTTCACCGACAAGATCATCCGCAG
CAACGCCACCGTGGAGCACCTGCACCCCATGGGCGATAACGTGCTGGTGG
GCAGCTTCGCCCGCACCTTCAGCCTGCGCGACGGCGGCTACTACAGCTTC
GTGGTGGACAGCCACATGCACTTCAAGAGCGCCATCCACCCCAGCATCCT
GCAGAACGGGGGCCCCATGTTCGCCTTCCGCCGCGTGGAGGAGCTGCACA
GCAACACCGAGCTGGGCATCGTGGAGTACCAGCACGCCTTCAAGACCCCC
ATCGCCTTCGCCAGATCCCGCGCTCAGTCGTCCAATTCTGCCGTGGACGG
CACCGCCGGACCCGGCTCCACCGGATCTCGCTAGAGCTGAATCtaagtcg
acaatcaacctctggattacaaaatttgtgaaagattgactggtattctt
aactatgttgctccttttacgctatgtggatacgctgctttaatgccttt
gtatcatgctattgcttcccgtatggctttcattttctcctccttgtata
aatcctggttgctgtctctttatgaggagttgtggcccgttgtcaggcaa
cgtggcgtggtgtgcactgtgtttgctgacgcaacccccactggttgggg
cattgccaccacctgtcagctcctttccgggactttcgctttccccctcc
ctattgccacggcggaactcatcgccgcctgccttgcccgctgctggaca
ggggctcggctgttgggcactgacaattccgtggtgttgtcggggaaatc
atcgtcctttccttggctgctcgcctgtgttgccacctggattctgcgcg
ggacgtccttctgctacgtcccttcggccctcaatccagcggaccttcct
tcccgcggcctgctgccggctctgcggcctcttccgcgtcttcgccttcg
ccctcagacgagtcggatctccctttgggccgcctccccgcccgggACAC
ACCAAGGATAATTTCTCCCGATACACACCAAGGATAATTTCTCCTCACAC
ACACCAAGGATAATTTCTCCCGATACACACCAAGGATAATTTCTCCTCAC
AACTGGATGTCCCTGTATGATTCGATAACTGGATGTCCCTGTATGATTTC
ACAACTGGATGTCCCTGTATGATTCGATAACTGGATGTCCCTGTATGATT
TCACAAAGAGGTTAACCATGTATTATCGATAAAGAGGTTAACCATGTATT
ATTCACAAAGAGGTTAACCATGTATTATCGATAAAGAGGTTAACCATGTA
TTATTCACGGTTAGTGGACCGTGTTACATACGATGGTTAGTGGACCGTGT
TACATATCACGGTTAGTGGACCGTGTTACATACGATGGTTAGTGGACCGT
GTTACATATCACGAATTCACCAAGGGCAACCTCTCGATGAATTCACCAAG
GGCAACCTCTTCACGAATTCACCAAGGGCAACCTCTCGATGAATTCACCA
AGGGCAACCTCTTCACAACTCCTGTATGAAGCCGTTCCGATAACTCCTGT
ATGAAGCCGTTCTCACAACTCCTGTATGAAGCCGTTCCGATAACTCCTGT
ATGAAGCCGTTCTCACATGGCAATATGTTGGCATAGCACGATATGGCAAT
ATGTTGGCATAGCATCACATGGCAATATGTTGGCATAGCACGATATGGCA
ATATGTTGGCATAGCATCACGCACAGCAAGTGTAGACAGGCACGATGCAC
AGCAAGTGTAGACAGGCATCACGCACAGCAAGTGTAGACAGGCACGATGC
ACAGCAAGTGTAGACAGGCATCACAACATACAAAGGGTATCCTCTCGATA
ACATACAAAGGGTATCCTCTTCACAACATACAAAGGGTATCCTCTCGATA
ACATACAAAGGGTATCCTCTTCACCTCCCTTCTTTCCTCCCGTCTTCGAT
CTCCCTTCTTTCCTCCCGTCTTTCACCTCCCTTCTTTCCTCCCGTCTTCG
ATCTCCCTTCTTTCCTCCCGTCTTcccgggtacctttaagaccaatgact
tacaaggcagctgtagatcttagccactttttaaaagaaaaggggggact
ggaagggctaattcactcccaacgaaaataagatctgctttttgcttgta
ctgggtctctctggttagaccagatctgagcctgggagctctctggctaa
ctagggaacccactgcttaagcctcaataaagcttgccttgagtgcttca
agtagtgtgtgcccgtctgttgtgtgactctggtaactagagatccctca
gacccttttagtcagtgtggaaaatctctagcagtagtagttcatgtcat
cttattattcagtatttataacttgcaaagaaatgaatatcagagagtga
gaggaacttgtttattgcagcttataatggttacaaataaagcaatagca
tcacaaatttcacaaataaagcatttttttcactgcattctagttgtggt
ttgtccaaactcatcaatgtatcttatcatgtctggctctagctatcccg
cccctaactccgcccagttccgcccattctccgccccatggctgactaat
tttttttatttatgcagaggccgaggccgcctcggcctctgagctattcc
agaagtagtgaggaggcttttttggaggcctagacttttgcagagacggc
ccaaattcgtaatcatggtcatagctgtttcctgtgtgaaattgttatcc
gctcacaattccacacaacatacgagccggaagcataaagtgtaaagcct
ggggtgcctaatgagtgagctaactcacattaattgcgttgcgctcactg
cccgctttccagtcgggaaacctgtcgtgccagctgcattaatgaatcgg
ccaacgcgcggggagaggcggtttgcgtattgggcgctcttccgcttcct
cgctcactgactcgctgcgctcggtcgttcggctgcggcgagcggtatca
gctcactcaaaggcggtaatacggttatccacagaatcaggggataacgc
aggaaagaacatgtgagcaaaaggccagcaaaaggccaggaaccgtaaaa
aggccgcgttgctggcgtttttccataggctccgcccccctgacgagcat
cacaaaaatcgacgctcaagtcagaggtggcgaaacccgacaggactata
aagataccaggcgtttccccctggaagctccctcgtgcgctctcctgttc
cgaccctgccgcttaccggatacctgtccgcctttctcccttcgggaagc
gtggcgctttctcatagctcacgctgtaggtatctcagttcggtgtaggt
cgttcgctccaagctgggctgtgtgcacgaaccccccgttcagcccgacc
gctgcgccttatccggtaactatcgtcttgagtccaacccggtaagacac
gacttatcgccactggcagcagccactggtaacaggattagcagagcgag
gtatgtaggcggtgctacagagttcttgaagtggtggcctaactacggct
acactagaaggacagtatttggtatctgcgctctgctgaagccagttacc
ttcggaaaaagagttggtagctcttgatccggcaaacaaaccaccgctgg
tagcggtggtttttttgtttgcaagcagcagattacgcgcagaaaaaaag
gatctcaagaagatcctttgatcttttctacggggtctgacgctcagtgg
aacgaaaactcacgttaagggattttggtcatgagattatcaaaaaggat
cttcacctagatccttttaaattaaaaatgaagttttaaatcaatctaaa
gtatatatgagtaaacttggtctgacagttaccaatgcttaatcagtgag
gcacctatctcagcgatctgtctatttcgttcatccatagttgcctgact
ccccgtcgtgtagataactacgatacgggagggcttaccatctggcccca
gtgctgcaatgataccgcgagacccacgctcaccggctccagatttatca
gcaataaaccagccagccggaagggccgagcgcagaagtggtcctgcaac
tttatccgcctccatccagtctattaattgttgccgggaagctagagtaa
gtagttcgccagttaatagtttgcgcaacgttgttgccattgctacaggc
atcgtggtgtcacgctcgtcgtttggtatggcttcattcagctccggttc
ccaacgatcaaggcgagttacatgatcccccatgttgtgcaaaaaagcgg
ttagctccttcggtcctccgatcgttgtcagaagtaagttggccgcagtg
ttatcactcatggttatggcagcactgcataattctcttactgtcatgcc
atccgtaagatgcttttctgtgactggtgagtactcaaccaagtcattct
gagaatagtgtatgcggcgaccgagttgctcttgcccggcgtcaatacgg
gataataccgcgccacatagcagaactttaaaagtgctcatcattggaaa
acgttcttcggggcgaaaactctcaaggatcttaccgctgttgagatcca
gttcgatgtaacccactcgtgcacccaactgatcttcagcatcttttact
ttcaccagcgtttctgggtgagcaaaaacaggaaggcaaaatgccgcaaa
aaagggaataagggcgacacggaaatgttgaatactcatactcttccttt
ttcaatattattgaagcatttatcagggttattgtctcatgagcggatac
atatttgaatgtatttagaaaaataaacaaataggggttccgcgcacatt
tccccgaaaagtgccacctgacgtctaagaaaccattattatcatgacat
taacctataaaaataggcgtatcacgaggccctttcgtctcgcgcgtttc
ggtgatgacggtgaaaacctctgacacatgcagctcccggagacggtcac
agcttgtctgtaagcggatgccgggagcagacaagcccgtcagggcgcgt
cagcgggtgttggcgggtgtcggggctggcttaactatgcggcatcagag
cagattgtactgagagtgcaccatatgcggtgtgaaataccgcacagatg
cgtaaggagaaaataccgcatcaggcgccattcgccattcaggctgcgca
actgttgggaagggcgatcggtgcgggcctcttcgctattacgccagctg
gcgaaagggggatgtgctgcaaggcgattaagttgggtaacgccagggtt
ttcccagtcacgacgttgtaaaacgacggccagtgccaagctg
[SEQ ID No: 81]

Preferably, the genetic construct comprises a nucleic acid sequence substantially as set out in SEQ ID No: 81, or a fragment or variant thereof.

Another embodiment of the genetic construct is shown in FIG. 8G and may comprise a Bim encoding sequence and be represented by SEQ ID No: 27, as follows:

acgcgtgtagtcttatgcaatactcttgtagtcttgcaacatggtaacga
tgagttagcaacatgccttacaaggagagaaaaagcaccgtgcatgccga
ttggtggaagtaaggtggtacgatcgtgccttattaggaaggcaacagac
gggtctgacatggattggacgaaccactgaattgccgcattgcagagata
ttgtatttaagtgcctagctcgatacaataaacgggtctctctggttaga
ccagatctgagcctgggagctctctggctaactagggaacccactgctta
agcctcaataaagcttgccttgagtgcttcaagtagtgtgtgcccgtctg
ttgtgtgactctggtaactagagatccctcagacccttttagtcagtgtg
gaaaatctctagcagtggcgcccgaacagggacctgaaagcgaaagggaa
accagagctctctcgacgcaggactcggcttgctgaagcgcgcacggcaa
gaggcgaggggcggcgactggtgagtacgccaaaaattttgactagcgga
ggctagaaggagagagatgggtgcgagagcgtcagtattaagcgggggag
aattagatcgcgatgggaaaaaattcggttaaggccagggggaaagaaaa
aatataaattaaaacatatagtatgggcaagcagggagctagaacgattc
gcagttaatcctggcctgttagaaacatcagaaggctgtagacaaatact
gggacagctacaaccatcccttcagacaggatcagaagaacttagatcat
tatataatacagtagcaaccctctattgtgtgcatcaaaggatagagata
aaagacaccaaggaagctttagacaagatagaggaagagcaaaacaaaag
taagaccaccgcacagcaagcggccactgatcttcagacctggaggagga
gatatgagggacaattggagaagtgaattatataaatataaagtagtaaa
aattgaaccattaggagtagcacccaccaaggcaaagagaagagtggtgc
agagagaaaaaagagcagtgggaataggagctttgttccttgggttcttg
ggagcagcaggaagcactatgggcgcagcctcaatgacgctgacggtaca
ggccagacaattattgtctggtatagtgcagcagcagaacaatttgctga
gggctattgaggcgcaacagcatctgttgcaactcacagtctggggcatc
aagcagctccaggcaagaatcctggctgtggaaagatacctaaaggatca
acagctcctggggatttggggttgctctggaaaactcatttgcaccactg
ctgtgccttggaatgctagttggagtaataaatctctggaacagattTgg
aatcacacgacctggatggagtgggacagagaaattaacaattacacaag
cttaatacactccttaattgaagaatcgcaaaaccagcaagaaaagaatg
aacaagaattattggaattagataaatgggcaagtttgtggaattggttt
aacataacaaattggetgtggtatataaaattattcataatgatagtagg
aggcttggtaggtttaagaatagtttttgctgtactttctatagtgaata
gagttaggcagggatattcaccattatcgtttcagacccacctcccaacc
ccgaggggacccgacaggcccgaaggaatagaagaagaaggtggagagag
agacagagacagatccattcgattagtgaacggatctcgacggtATCGGT
taacttttaaaagaaaaggggggattggggggtacagtgcaggggaaaga
atagtagacataatagcaacagacatacaaactaaagaattacaaaaaca
aattacaaaaattcaaaattttatGTCGAGccatagagcccaccgcatcc
ccagcatgcctgctattgtcttcccaatcctcccccttgctgtcctgccc
caccccaccccccagaatagaatgacacctactcagacaatgcgatgcaa
tttcctcattttattaggaaaggacagtgggagtggcaccttccagggtc
aaggaaggcacgggggaggggcaaacaacagatggctggcaactagaagg
cacagtcgCtcgagAGTAAAACACTACAACGTCGATAGCAGTAAAACACT
ACAACGTCGACACTAGTAAAACACTACAACGTCGATAGCAGTAAAACACT
ACAACGTCGACACTAGTGTCACTTGGCCAGAGAAATAGCAGTGTCACTTG
GCCAGAGAAACACTAGTGTCACTTGGCCAGAGAAATAGCAGTGTCACTTG
GCCAGAGAAActcgagTCAAACCTTCCTCTTCTTCTTAGGAGGCCTCAGG
CTGCTCTGCCCGCTTTCCAGTCGGGAAACCTGTCGTGCCAGCTGCATTAA
TGAATCGGCCAACGCGCGGGGAGAGGCGGTTTGCGTATTGGGCGCCAGGG
TGGTTTTTCTTTTCACCAGTGAGACGGGCAACAGCTGATTGCCCTTCACC
GCCTGGCCCTGAGAGAGTTGCAGCAAGCGGTCCACGCTGGTTTGCCCCAG
CAGGCGAAAATCCTGTTTGATGGTGGTTAACGGCGGGATATAACATGAGC
TGTCTTCGGTATCGTCGTATCCCACTACCGAGATATCCGCACCAACGCGC
AGCCCGGACTCGGTAATGGCGCGCATTGCGCCCAGCGCCATCTGATCGTT
GGCAACCAGCATCGCAGTGGGAACGATGCCCTCATTCAGCATTTGCATGG
TTTGTTGAAAACCGGACATGGCACTCCAGTCGCCTTCCCGTTCCGCTATC
GGCTGAATTTGATTGCGAGTGAGATATTTATGCCAGCCAGCCAGACGCAG
ACGCGCCGAGACAGAACTTAATGGGCCCGCTAACAGCGCGATTTGCTGGT
GACCCAATGCGACCAGATGCTCCACGCCCAGTCGCGTACCGTCTTCATGG
GAGAAAATAATACTGTTGATGGGTGTCTGGTCAGAGACATCAAGAAATAA
CGCCGGAACATTAGTGCAGGCAGCTTCCACAGCAATGGCATCCTGGTCAT
CCAGCGGATAGTTAATGATCAGCCCACTGACGCGTTGCGCGAGAAGATTG
TGCACCGCCGCTTTACAGGCTTCGACGCCGCTTCGTTCTACCATCGACAC
CACCACGCTGGCACCCAGTTGATCGGCGCGAGATTTAATCGCCGCGACAA
TTTGCGACGGCGCGTGCAGGGCCAGACTGGAGGTGGCAACGCCAATCAGC
AACGACTGTTTGCCCGCCAGTTGTTGTGCCACGCGGTTGGGAATGTAATT
CAGCTCCGCCATCGCCGCTTCCACTTTTTCCCGCGTTTTCGCAGAAACGT
GGCTGGCCTGGTTCACCACGCGGGAAACGGTCTGATAAGAGACACCGGCA
TACTCTGCGACATCGTATAACGTTACTGGTTTCATGGTGGGAGGGTACcT
AGGTAAGCTTGGGCTGCAGGTCGAAAGGCCCGGAGATGAGGAAGAGGAGA
ACAGCGCGGCAGACGTGCGCTTTTGAAGCGTGCAGAATGCCGGGCCTCCG
GAGGACCTTCGGGCGCCCGCCCCGCCCCTGAGCCCGCCCCTGAGCCCGCC
CCCGGACCCACCCCTTCCCAGCCTCTGAGCCCAGAAAGCGAAGGAGCAAA
GCTGCTATTGGCCGCTGCCCCAAAGGCCTACCCGCTTCCATTGCTCAGCG
GTGCTGTCCATCTGCACGAGACTAGTGAGACGTGCTACTTCCATTTGTCA
CGTCCTGCACGACGCGAGCTGCGGGGCGGGGGGGAACTTCCTGACTAGGG
GAGGAGTAGAAGGTGGCGCGAAGGGGCCACCAAAGAACGGAGCCGGTTGG
CGCCTACCGGTGGATGTGGAATGTGTGCGAGGCCAGAGGCCACTTGTGTA
GCGCCAAGTGCCCAGCGGGGCTGCTAAAGCGCATGCTCCAGACTGCCTTG
GGAAAAGCGCCTCCCCTACCCGGTAGAAGCTAGCGGCTCCGGTGCCCGTC
AGTGGGCAGAGCGCACATCGCCCACAGTCCCCGAGAAGTTGTGGGGAGGG
GTCGGCAATTGAACCGGTGCCTAGAGAAGGTGGCGCGGGGTAAACTGGGA
AAGTGATGTCGTGTACTGGCTCCGCCTTTTTCCCGAGGGTGGGGGAGAAC
CGTATATAAGTGCAGTAGTCGCCGTGAACGTTCTTTTTCGCAACGGGTTT
GCCGCCAGAACACAGGTAAGTGCCGTGTGTGGTTCCCGCGGGCCTGGCCT
CTTTACGGGTTATGGCCCTTGCGTGCCTTGAATTACTTCCACCTGGCTGC
AGTACGTGATTCTTGATCCCGAGCTTCGGGTTGGAAGTGGGTGGGAGAGT
TCGAGGCCTTGCGCTTAAGGAGCCCCTTCGCCTCGTGCTTGAGTTGAGGC
CTGGCCTGGGCGCTGGGGCCGCCGCGTGCGAATCTGGTGGCACCTTCGCG
CCTGTCTCGCTGCTTTCGATAAGTCTCTAGCCATTTAAAATTTTTGATGA
CCTGCTGCGACGCTTTTTTTCTGGCAAGATAGTCTTGTAAATGCGGGCCA
AGATCTGCACACTGGTATTTCGGTTTTTGGGGCCGCGGGCGGCGACGGGG
CCCGTGCGTCCCAGCGCACATGTTCGGCGAGGCGGGGCCTGCGAGCGCGG
CCACCGAGAATCGGACGGGGGTAGTCTCAAGCTGGCCGGCCTGCTCTGGT
GCCTGGCCTCGCGCCGCCGTGTATCGCCCCGCCCTGGGCGGCAAGGCTGG
CCCGGTCGGCACCAGTTGCGTGAGCGGAAAGATGGCCGCTTCCCGGCCCT
GCTGCAGGGAGCTCAAAATGGAGGACGCGGCGCTCGGGAGAGCGGGCGGG
TGAGTCACCCACACAAAGGAAAAGGGCCTTTCCGTCCTCAGCCGTCGCTT
CATGTGACTCCACGGAGTACCGGGCGCCGTCCAGGCACCTCGATTAGTTC
TCGACCTTTTGGAGTACGTCGTCTTTAGGTTGGGGGGAGGGGTTTTATGC
GATGGAGTTTCCCCACACTGAGTGGGTGGAGACTGAAGTTAGGCCAGCTT
GGCACTTGATGTAATTCTCCTTGGAATTTGCCCTTTTTGAGTTTGGATCT
TGGTTCATTCTCAAGCCTCAGACAGTGGTTCAAAGTTTTTTTCTTCCATT
TCAGGTGTCGTGAtctagagctagcgAATTCGTAAATATAAAATTTACTA
GGTTGTGGAATTGTGAGCGCTCACAATTCCACAGTCGACCCTAGGTTGTG
GAATTGTGAGCGCTCACAATTCCACAGTCGACCCTAGGTTGTGGAATTGT
GAGCGCTCACAATTCCACAGTCGACCCTAGTGTATAATGTGTTAAACTAC
GGATCCGTCTCCCATTAGGCCTACAATGGTGAGACAAGTAGCCAACAGGG
AAGGGTTGCAAATATCATTTGGGCACACCTATGATAATATTGATGAAGCA
GACAGTATTCAGCAAGTAACTGAGAGGTGGGAAGCTCAAAGCCAAAGTCC
TAATGTGCAGTCAGGTGAATTTATTGAAAAATTTGAGGCTCCTGGTGGTG
CAAATCAAAGAACTGCTCCTCAGGGATCCTAATTGTTTGTGTATTTTAGG
Cggccgcatggcaaagcaaccttctgatgtaagttctgagtgtgaccgag
aaggtagacaattgcagcctgcggagaggcctccccagctcagacctggg
gcccctacctccctacagacagagccacaaggtaatcctgaaggcaatca
cggaggtgaaggggacagctgcccccacggcagccctcagggcccgctgg
ccccacctgccagccctggcccttttgctaccagatccccgcttttcatc
tttatgagaagatcctccctgctgtctcgatcctccagtgggtatttctc
ttttgacacagacaggagcccagcacccatgagttgtgacaaatcaacac
aaaccccaagtcctccttgccaggccttcaaccactatctcagtgcaatg
gcttccatgaggcaggctgaacctgcagatatgcgcccagagatatggat
cgcccaagagttgcggcgtattggagacgagtttaacgcttactatgcaa
ggagggtatttttgaataattaccaagcagccgaagaccacccacgaatg
gttatcttacgactgttacgttacattgtccgcctggtgtggagaatgca
ttgaGCGGCCGCtgagggcagaggaagtcttctaacatgcggtgacgtgg
aggagaatcccggcccttccggaATGGAGAGCGACGAGAGCGGCCTGCCC
GCCATGGAGATCGAGTGCCGCATCACCGGCACCCTGAACGGCGTGGAGTT
CGAGCTGGTGGGCGGCGGAGAGGGCACCCCCAAGCAGGGCCGCATGACCA
ACAAGATGAAGAGCACCAAAGGCGCCCTGACCTTCAGCCCCTACCTGCTG
AGCCACGTGATGGGCTACGGCTTCTACCACTTCGGCACCTACCCCAGCGG
CTACGAGAACCCCTTCCTGCACGCCATCAACAACGGCGGCTACACCAACA
CCCGCATCGAGAAGTACGAGGACGGCGGCGTGCTGCACGTGAGCTTCAGC
TACCGCTACGAGGCCGGCCGCGTGATCGGCGACTTCAAGGTGGTGGGCAC
CGGCTTCCCCGAGGACAGCGTGATCTTCACCGACAAGATCATCCGCAGCA
ACGCCACCGTGGAGCACCTGCACCCCATGGGCGATAACGTGCTGGTGGGC
AGCTTCGCCCGCACCTTCAGCCTGCGCGACGGCGGCTACTACAGCTTCGT
GGTGGACAGCCACATGCACTTCAAGAGCGCCATCCACCCCAGCATCCTGC
AGAACGGGGGCCCCATGTTCGCCTTCCGCCGCGTGGAGGAGCTGCACAGC
AACACCGAGCTGGGCATCGTGGAGTACCAGCACGCCTTCAAGACCCCCAT
CGCCTTCGCCAGATCCCGCGCTCAGTCGTCCAATTCTGCCGTGGACGGCA
CCGCCGGACCCGGCTCCACCGGATCTCGCTAGAGCTGAATCtaagtcgac
aatcaacctctggattacaaaatttgtgaaagattgactggtattcttaa
ctatgttgctccttttacgctatgtggatacgctgctttaatgcctttgt
atcatgctattgcttcccgtatggctttcattttctcctccttgtataaa
tcctggttgctgtctctttatgaggagttgtggcccgttgtcaggcaacg
tggcgtggtgtgcactgtgtttgctgacgcaacccccactggttggggca
ttgccaccacctgtcagctcctttccgggactttcgctttccccctccct
attgccacggcggaactcatcgccgcctgccttgcccgctgctggacagg
ggctcggctgttgggcactgacaattccgtggtgttgtcggggaaatcat
cgtcctttccttggctgctcgcctgtgttgccacctggattctgcgcggg
acgtccttctgctacgtcccttcggccctcaatccagcggaccttccttc
ccgcggcctgctgccggctctgcggcctcttccgcgtcttcgccttcgcc
ctcagacgagtcggatctccctttgggccgcctccccgcccgggTAACCG
ATTTCAGATGGTGCTACGATTAACCGATTTCAGATGGTGCTATCACTAAC
CGATTTCAGATGGTGCTACGATTAACCGATTTCAGATGGTGCTATCACGG
GAGTGAAGACACGGAGCCAGACGATGGGAGTGAAGACACGGAGCCAGATC
ACGGGAGTGAAGACACGGAGCCAGACGATGGGAGTGAAGACACGGAGCCA
GAcccgggtacctttaagaccaatgacttacaaggcagctgtagatctta
gccactttttaaaagaaaaggggggactggaagggctaattcactcccaa
cgaaaataagatctgcttfttgcttgtactgggtctctctggttagacca
gatctgagcctgggagctctctggctaactagggaacccactgcttaagc
ctcaataaagcttgccttgagtgcttcaagtagtgtgtgcccgtctgttg
tgtgactctggtaactagagatccctcagacccttttagtcagtgtggaa
aatctctagcagtagtagttcatgtcatcttattattcagtatttataac
ttgcaaagaaatgaatatcagagagtgagaggaacttgfttattgcagct
tataatggttacaaataaagcaatagcatcacaaatttcacaaataaagc
atttttttcactgcattctagttgtggtttgtccaaactcatcaatgtat
cttatcatgtctggctctagctatcccgcccctaactccgcccagttccg
cccattctccgccccatggctgactaattttttttatttatgcagaggcc
gaggccgcctcggcctctgagctattccagaagtagtgaggaggcttttt
tggaggcctagacttttgcagagacggcccaaattcgtaatcatggtcat
agctgtttcctgtgtgaaattgttatccgctcacaattccacacaacata
cgagccggaagcataaagtgtaaagcctggggtgcctaatgagtgagcta
actcacattaattgcgttgcgctcactgcccgctttccagtcgggaaacc
tgtcgtgccagctgcattaatgaatcggccaacgcgcggggagaggcggt
ttgcgtattgggcgctcttccgcttcctcgctcactgactcgctgcgctc
ggtcgttcggctgcggcgagcggtatcagctcactcaaaggcggtaatac
ggttatccacagaatcaggggataacgcaggaaagaacatgtgagcaaaa
ggccagcaaaaggccaggaaccgtaaaaaggccgcgttgctggcgttttt
ccataggctccgcccccctgacgagcatcacaaaaatcgacgctcaagtc
agaggtggcgaaacccgacaggactataaagataccaggcgtttccccct
ggaagctccctcgtgcgctctcctgttccgaccctgccgcttaccggata
cctgtccgcctttctcccttcgggaagcgtggcgctttctcatagctcac
gctgtaggtatctcagttcggtgtaggtcgttcgctccaagctgggctgt
gtgcacgaaccccccgttcagcccgaccgctgcgccttatccggtaacta
tcgtcttgagtccaacccggtaagacacgacttatcgccactggcagcag
ccactggtaacaggattagcagagcgaggtatgtaggcggtgctacagag
ttcttgaagtggtggcctaactacggctacactagaaggacagtatttgg
tatctgcgctctgctgaagccagttaccttcggaaaaagagttggtagct
cttgatccggcaaacaaaccaccgetggtagcggtggtttttttgtttgc
aagcagcagattacgcgcagaaaaaaaggatctcaagaagatcctttgat
cttttctacggggtctgacgctcagtggaacgaaaactcacgttaaggga
ttttggtcatgagattatcaaaaaggatcttcacctagatccttttaaat
taaaaatgaagttttaaatcaatctaaagtatatatgagtaaacttggtc
tgacagttaccaatgcttaatcagtgaggcacctatctcagcgatctgtc
tatttcgttcatccatagttgcctgactccccgtcgtgtagataactacg
atacgggagggcttaccatctggccccagtgctgcaatgataccgcgaga
cccacgctcaccggctccagatttatcagcaataaaccagccagccggaa
gggccgagcgcagaagtggtcctgcaactttatccgcctccatccagtct
attaattgttgccgggaagctagagtaagtagttcgccagttaatagttt
gcgcaacgttgttgccattgctacaggcatcgtggtgtcacgctcgtcgt
ttggtatggcttcattcagctccggttcccaacgatcaaggcgagttaca
tgatcccccatgttgtgcaaaaaagcggttagctccttcggtcctccgat
cgttgtcagaagtaagttggccgcagtgttatcactcatggttatggcag
cactgcataattctcttactgtcatgccatccgtaagatgcttttctgtg
actggtgagtactcaaccaagtcattctgagaatagtgtatgcggcgacc
gagttgctcttgcccggcgtcaatacgggataataccgcgccacatagca
gaactttaaaagtgctcatcattggaaaacgttcttcggggcgaaaactc
tcaaggatcttaccgctgttgagatccagttcgatgtaacccactcgtgc
acccaactgatcttcagcatcttttactttcaccagcgtttctgggtgag
caaaaacaggaaggcaaaatgccgcaaaaaagggaataagggcgacacgg
aaatgttgaatactcatactcttcctttttcaatattattgaagcattta
tcagggttattgtctcatgagcggatacatatttgaatgtatttagaaaa
ataaacaaataggggttccgcgcacatttccccgaaaagtgccacctgac
gtctaagaaaccattattatcatgacattaacctataaaaataggcgtat
cacgaggccctttcgtctcgcgcgtttcggtgatgacggtgaaaacctct
gacacatgcagctcccggagacggtcacagcttgtctgtaagcggatgcc
gggagcagacaagcccgtcagggcgcgtcagcgggtgttggcgggtgtcg
gggctggcttaactatgcggcatcagagcagattgtactgagagtgcacc
atatgcggtgtgaaataccgcacagatgcgtaaggagaaaataccgcatc
aggcgccattcgccattcaggctgcgcaactgttgggaagggcgatcggt
gcgggcctcttcgctattacgccagctggcgaaagggggatgtgctgcaa
ggcgattaagttgggtaacgccagggttttcccagtcacgacgttgtaaa
acgacggccagtgccaagctg
[SEQ ID N
o: 27]

Preferably, the genetic construct comprises a nucleic acid sequence substantially as set out in SEQ ID No: 27, or a fragment or variant thereof.

Another embodiment of the genetic construct comprising a Bim encoding sequence is shown in FIG. 8L and is represented by SEQ ID No: 82, as follows:

acgcgtgtagtcttatgcaatactcttgtagtcttgcaacatggtaacga
tgagttagcaacatgccttacaaggagagaaaaagcaccgtgcatgccga
ttggtggaagtaaggtggtacgatcgtgccttattaggaaggcaacagac
gggtctgacatggattggacgaaccactgaattgccgcattgcagagata
ttgtatttaagtgcctagctcgatacaataaacgggtctctctggttaga
ccagatctgagcctgggagctctctggctaactagggaacccactgctta
agcctcaataaagcttgccttgagtgcttcaagtagtgtgtgcccgtctg
ttgtgtgactctggtaactagagatccctcagacccttttagtcagtgtg
gaaaatctctagcagtggcgcccgaacagggacctgaaagcgaaagggaa
accagagctctctcgacgcaggactcggcttgctgaagcgcgcacggcaa
gaggcgaggggcggcgactggtgagtacgccaaaaattttgactagcgga
ggctagaaggagagagatgggtgcgagagcgtcagtattaagcgggggag
aattagatcgcgatgggaaaaaattcggttaaggccagggggaaagaaaa
aatataaattaaaacatatagtatgggcaagcagggagctagaacgattc
gcagttaatcctggcctgttagaaacatcagaaggctgtagacaaatact
gggacagctacaaccatcccttcagacaggatcagaagaacttagatcat
tatataatacagtagcaaccctctattgtgtgcatcaaaggatagagata
aaagacaccaaggaagctttagacaagatagaggaagagcaaaacaaaag
taagaccaccgcacagcaagcggccactgatcttcagacctggaggagga
gatatgagggacaattggagaagtgaattatataaatataaagtagtaaa
aattgaaccattaggagtagcacccaccaaggcaaagagaagagtggtgc
agagagaaaaaagagcagtgggaataggagctttgttccttgggttcttg
ggagcagcaggaagcactatgggcgcagcctcaatgacgctgacggtaca
ggccagacaattattgtctggtatagtgcagcagcagaacaatttgctga
gggctattgaggcgcaacagcatctgttgcaactcacagtctggggcatc
aagcagctccaggcaagaatcctggctgtggaaagatacctaaaggatca
acagctcctggggatttggggttgctctggaaaactcatttgcaccactg
ctgtgccttggaatgctagttggagtaataaatctctggaacagattTgg
aatcacacgacctggatggagtgggacagagaaattaacaattacacaag
cttaatacactccttaattgaagaatcgcaaaaccagcaagaaaagaatg
aacaagaattattggaattagataaatgggcaagtttgtggaattggttt
aacataacaaattggctgtggtatataaaattattcataatgatagtagg
aggcttggtaggtttaagaatagtttttgctgtactttctatagtgaata
gagttaggcagggatattcaccattatcgtttcagacccacctcccaacc
ccgaggggacccgacaggcccgaaggaatagaagaagaaggtggagagag
agacagagacagatccattcgattagtgaacggatctcgacggtATCGGT
taacttttaaaagaaaaggggggattggggggtacagtgcaggggaaaga
atagtagacataatagcaacagacatacaaactaaagaattacaaaaaca
aattacaaaaattcaaaattttatGTCGAGccatagagcccaccgcatcc
ccagcatgcctgctattgtcttcccaatcctcccccttgctgtcctgccc
caccccaccccccagaatagaatgacacctactcagacaatgcgatgcaa
tttcctcattttattaggaaaggacagtgggagtggcaccttccagggtc
aaggaaggcacgggggaggggcaaacaacagatggctggcaactagaagg
cacagtcgCtcgagGGGCCTGTCCGCAAGCACGCTGCATAGCGGGCCTGT
CCGCAAGCACGCTGCACACTGGGCCTGTCCGCAAGCACGCTGCATAGCGG
GCCTGTCCGCAAGCACGCTGCACACTCCCACCCCTAAACAACGTAATGTA
GCCCCACCCCTAAACAACGTAATGCACTCCCACCCCTAAACAACGTAATG
TAGCCCCACCCCTAAACAACGTAATGCACTTGACATCATACCCGTGAAGG
TCTAGCTGACATCATACCCGTGAAGGTCCACTTGACATCATACCCGTGAA
GGTCTAGCTGACATCATACCCGTGAAGGTCCACTACTCCGCCCCCCCGCT
CGTAGCACTCCGCCCCCCCGCTCGCACTACTCCGCCCCCCCGCTCGTAGC
ACTCCGCCCCCCCGCTCGCACTGACGTTACATTCGTGAAGAATGTAGCGA
CGTTACATTCGTGAAGAATGCACTGACGTTACATTCGTGAAGAATGTAGC
GACGTTACATTCGTGAAGAATGCACTGCCCCAGCCGCCGCTGCACTAGCG
CCCCAGCCGCCGCTGCACCACTGCCCCAGCCGCCGCTGCACTAGCGCCCC
AGCCGCCGCTGCACctcgagTCAAACCTTCCTCTTCTTCTTAGGAGGCCT
CAGGCTGCTCTGCCCGCTTTCCAGTCGGGAAACCTGTCGTGCCAGCTGCA
TTAATGAATCGGCCAACGCGCGGGGAGAGGCGGTTTGCGTATTGGGCGCC
AGGGTGGTTTTTCTTTTCACCAGTGAGACGGGCAACAGCTGATTGCCCTT
CACCGCCTGGCCCTGAGAGAGTTGCAGCAAGCGGTCCACGCTGGTTTGCC
CCAGCAGGCGAAAATCCTGTTTGATGGTGGTTAACGGCGGGATATAACAT
GAGCTGTCTTCGGTATCGTCGTATCCCACTACCGAGATATCCGCACCAAC
GCGCAGCCCGGACTCGGTAATGGCGCGCATTGCGCCCAGCGCCATCTGAT
CGTTGGCAACCAGCATCGCAGTGGGAACGATGCCCTCATTCAGCATTTGC
ATGGTTTGTTGAAAACCGGACATGGCACTCCAGTCGCCTTCCCGTTCCGC
TATCGGCTGAATTTGATTGCGAGTGAGATATTTATGCCAGCCAGCCAGAC
GCAGACGCGCCGAGACAGAACTTAATGGGCCCGCTAACAGCGCGATTTGC
TGGTGACCCAATGCGACCAGATGCTCCACGCCCAGTCGCGTACCGTCTTC
ATGGGAGAAAATAATACTGTTGATGGGTGTCTGGTCAGAGACATCAAGAA
ATAACGCCGGAACATTAGTGCAGGCAGCTTCCACAGCAATGGCATCCTGG
TCATCCAGCGGATAGTTAATGATCAGCCCACTGACGCGTTGCGCGAGAAG
ATTGTGCACCGCCGCTTTACAGGCTTCGACGCCGCTTCGTTCTACCATCG
ACACCACCACGCTGGCACCCAGTTGATCGGCGCGAGATTTAATCGCCGCG
ACAATTTGCGACGGCGCGTGCAGGGCCAGACTGGAGGTGGCAACGCCAAT
CAGCAACGACTGTTTGCCCGCCAGTTGTTGTGCCACGCGGTTGGGAATGT
AATTCAGCTCCGCCATCGCCGCTTCCACTTTTTCCCGCGTTTTCGCAGAA
ACGTGGCTGGCCTGGTTCACCACGCGGGAAACGGTCTGATAAGAGACACC
GGCATACTCTGCGACATCGTATAACGTTACTGGTTTCATGGTGGGAGGGT
ACcTAGGTAAGCTTGGGCTGCAGGTCGAAAGGCCCGGAGATGAGGAAGAG
GAGAACAGCGCGGCAGACGTGCGCTTTTGAAGCGTGCAGAATGCCGGGCC
TCCGGAGGACCTTCGGGCGCCCGCCCCGCCCCTGAGCCCGCCCCTGAGCC
CGCCCCCGGACCCACCCCTTCCCAGCCTCTGAGCCCAGAAAGCGAAGGAG
CAAAGCTGCTATTGGCCGCTGCCCCAAAGGCCTACCCGCTTCCATTGCTC
AGCGGTGCTGTCCATCTGCACGAGACTAGTGAGACGTGCTACTTCCATTT
GTCACGTCCTGCACGACGCGAGCTGCGGGGCGGGGGGGAACTTCCTGACT
AGGGGAGGAGTAGAAGGTGGCGCGAAGGGGCCACCAAAGAACGGAGCCGG
TTGGCGCCTACCGGTGGATGTGGAATGTGTGCGAGGCCAGAGGCCACTTG
TGTAGCGCCAAGTGCCCAGCGGGGCTGCTAAAGCGCATGCTCCAGACTGC
CTTGGGAAAAGCGCCTCCCCTACCCGGTAGAAGCTAGCGGCTCCGGTGCC
CGTCAGTGGGCAGAGCGCACATCGCCCACAGTCCCCGAGAAGTTGTGGGG
AGGGGTCGGCAATTGAACCGGTGCCTAGAGAAGGTGGCGCGGGGTAAACT
GGGAAAGTGATGTCGTGTACTGGCTCCGCCTTTTTCCCGAGGGTGGGGGA
GAACCGTATATAAGTGCAGTAGTCGCCGTGAACGTTCTTTTTCGCAACGG
GTTTGCCGCCAGAACACAGGTAAGTGCCGTGTGTGGTTCCCGCGGGCCTG
GCCTCTTTACGGGTTATGGCCCTTGCGTGCCTTGAATTACTTCCACCTGG
CTGCAGTACGTGATTCTTGATCCCGAGCTTCGGGTTGGAAGTGGGTGGGA
GAGTTCGAGGCCTTGCGCTTAAGGAGCCCCTTCGCCTCGTGCTTGAGTTG
AGGCCTGGCCTGGGCGCTGGGGCCGCCGCGTGCGAATCTGGTGGCACCTT
CGCGCCTGTCTCGCTGCTTTCGATAAGTCTCTAGCCATTTAAAATTTTTG
ATGACCTGCTGCGACGCTTTTTTTCTGGCAAGATAGTCTTGTAAATGCGG
GCCAAGATCTGCACACTGGTATTTCGGTTTTTGGGGCCGCGGGCGGCGAC
GGGGCCCGTGCGTCCCAGCGCACATGTTCGGCGAGGCGGGGCCTGCGAGC
GCGGCCACCGAGAATCGGACGGGGGTAGTCTCAAGCTGGCCGGCCTGCTC
TGGTGCCTGGCCTCGCGCCGCCGTGTATCGCCCCGCCCTGGGCGGCAAGG
CTGGCCCGGTCGGCACCAGTTGCGTGAGCGGAAAGATGGCCGCTTCCCGG
CCCTGCTGCAGGGAGCTCAAAATGGAGGACGCGGCGCTCGGGAGAGCGGG
CGGGTGAGTCACCCACACAAAGGAAAAGGGCCTTTCCGTCCTCAGCCGTC
GCTTCATGTGACTCCACGGAGTACCGGGCGCCGTCCAGGCACCTCGATTA
GTTCTCGACCTTTTGGAGTACGTCGTCTTTAGGTTGGGGGGAGGGGTTTT
ATGCGATGGAGTTTCCCCACACTGAGTGGGTGGAGACTGAAGTTAGGCCA
GCTTGGCACTTGATGTAATTCTCCTTGGAATTTGCCCTTTTTGAGTTTGG
ATCTTGGTTCATTCTCAAGCCTCAGACAGTGGTTCAAAGTTTTTTTCTTC
CATTTCAGGTGTCGTGAtctagagctagcgAATTCGTAAATATAAAATTT
ACTAGGTTGTGGAATTGTGAGCGCTCACAATTCCACAGTCGACCCTAGGT
TGTGGAATTGTGAGCGCTCACAATTCCACAGTCGACCCTAGGTTGTGGAA
TTGTGAGCGCTCACAATTCCACAGTCGACCCTAGTGTATAATGTGTTAAA
CTACGGATCCGTCTCCCATTAGGCCTACAATGGTGAGACAAGTAGCCAAC
AGGGAAGGGTTGCAAATATCATTTGGGCACACCTATGATAATATTGATGA
AGCAGACAGTATTCAGCAAGTAACTGAGAGGTGGGAAGCTCAAAGCCAAA
GTCCTAATGTGCAGTCAGGTGAATTTATTGAAAAATTTGAGGCTCCTGGT
GGTGCAAATCAAAGAACTGCTCCTCAGGGATCCTAATTGTTTGTGTATTT
TAGGCggccgcatggcaaagcaaccttctgatgtaagttctgagtgtgac
cgagaaggtagacaattgcagcctgcggagaggcctccccagctcagacc
tggggcccctacctccctacagacagagccacaaggtaatcctgaaggca
atcacggaggtgaaggggacagctgcccccacggcagccctcagggcccg
ctggccccacctgccagccctggcccttttgctaccagatccccgctttt
catctttatgagaagatcctccctgctgtctcgatcctccagtgggtatt
tctcttttgacacagacaggagcccagcacccatgagttgtgacaaatca
acacaaaccccaagtcctccttgccaggccttcaaccactatctcagtgc
aatggcttccatgaggcaggctgaacctgcagatatgcgcccagagatat
ggatcgcccaagagttgcggcgtattggagacgagtttaacgcttactat
gcaaggagggtatttttgaataattaccaagcagccgaagaccacccacg
aatggttatcttacgactgttacgttacattgtccgcctggtgtggagaa
tgcattgagcggccgctgagggcagaggaagtcttctaacatgcggtgac
gtggaggagaatcccggcccttccggaATGGAGAGCGACGAGAGCGGCCT
GCCCGCCATGGAGATCGAGTGCCGCATCACCGGCACCCTGAACGGCGTGG
AGTTCGAGCTGGTGGGCGGCGGAGAGGGCACCCCCAAGCAGGGCCGCATG
ACCAACAAGATGAAGAGCACCAAAGGCGCCCTGACCTTCAGCCCCTACCT
GCTGAGCCACGTGATGGGCTACGGCTTCTACCACTTCGGCACCTACCCCA
GCGGCTACGAGAACCCCTTCCTGCACGCCATCAACAACGGCGGCTACACC
AACACCCGCATCGAGAAGTACGAGGACGGCGGCGTGCTGCACGTGAGCTT
CAGCTACCGCTACGAGGCCGGCCGCGTGATCGGCGACTTCAAGGTGGTGG
GCACCGGCTTCCCCGAGGACAGCGTGATCTTCACCGACAAGATCATCCGC
AGCAACGCCACCGTGGAGCACCTGCACCCCATGGGCGATAACGTGCTGGT
GGGCAGCTTCGCCCGCACCTTCAGCCTGCGCGACGGCGGCTACTACAGCT
TCGTGGTGGACAGCCACATGCACTTCAAGAGCGCCATCCACCCCAGCATC
CTGCAGAACGGGGGCCCCATGTTCGCCTTCCGCCGCGTGGAGGAGCTGCA
CAGCAACACCGAGCTGGGCATCGTGGAGTACCAGCACGCCTTCAAGACCC
CCATCGCCTTCGCCAGATCCCGCGCTCAGTCGTCCAATTCTGCCGTGGAC
GGCACCGCCGGACCCGGCTCCACCGGATCTCGCTAGAGCTGAATCtaagt
cgacaatcaacctctggattacaaaatttgtgaaagattgactggtattc
ttaactatgttgctccttttacgctatgtggatacgctgctttaatgcct
ttgtatcatgctattgcttcccgtatggctttcattttctcctccttgta
taaatcctggttgctgtctctttatgaggagttgtggcccgttgtcaggc
aacgtggcgtggtgtgcactgtgtttgctgacgcaacccccactggttgg
ggcattgccaccacctgtcagctcctttccgggactttcgctttccccct
ccctattgccacggcggaactcatcgccgcctgccttgcccgctgctgga
caggggctcggctgttgggcactgacaattccgtggtgttgtcggggaaa
tcatcgtcctttccttggctgctcgcctgtgttgccacctggattctgcg
cgggacgtccttctgctacgtcccttcggccctcaatccagcggaccttc
cttcccgcggcctgctgccggctctgcggcctcttccgcgtcttcgcctt
cgccctcagacgagtcggatctccctttgggccgcctccccgcccgggAC
ACACCAAGGATAATTTCTCCCGATACACACCAAGGATAATTTCTCCTCAC
ACACACCAAGGATAATTTCTCCCGATACACACCAAGGATAATTTCTCCTC
ACAACTGGATGTCCCTGTATGATTCGATAACTGGATGTCCCTGTATGATT
TCACAACTGGATGTCCCTGTATGATTCGATAACTGGATGTCCCTGTATGA
TTTCACAAAGAGGTTAACCATGTATTATCGATAAAGAGGTTAACCATGTA
TTATTCACAAAGAGGTTAACCATGTATTATCGATAAAGAGGTTAACCATG
TATTATTCACGGTTAGTGGACCGTGTTACATACGATGGTTAGTGGACCGT
GTTACATATCACGGTTAGTGGACCGTGTTACATACGATGGTTAGTGGACC
GTGTTACATATCACGAATTCACCAAGGGCAACCTCTCGATGAATTCACCA
AGGGCAACCTCTTCACGAATTCACCAAGGGCAACCTCTCGATGAATTCAC
CAAGGGCAACCTCTTCACAACTCCTGTATGAAGCCGTTCCGATAACTCCT
GTATGAAGCCGTTCTCACAACTCCTGTATGAAGCCGTTCCGATAACTCCT
GTATGAAGCCGTTCTCACATGGCAATATGTTGGCATAGCACGATATGGCA
ATATGTTGGCATAGCATCACATGGCAATATGTTGGCATAGCACGATATGG
CAATATGTTGGCATAGCATCACGCACAGCAAGTGTAGACAGGCACGATGC
ACAGCAAGTGTAGACAGGCATCACGCACAGCAAGTGTAGACAGGCACGAT
GCACAGCAAGTGTAGACAGGCATCACAACATACAAAGGGTATCCTCTCGA
TAACATACAAAGGGTATCCTCTTCACAACATACAAAGGGTATCCTCTCGA
TAACATACAAAGGGTATCCTCTTCACCTCCCTTCTTTCCTCCCGTCTTCG
ATCTCCCTTCTTTCCTCCCGTCTTTCACCTCCCTTCTTTCCTCCCGTCTT
CGATCTCCCTTCTTTCCTCCCGTCTTcccgggtacetttaagaccaatga
cttacaaggcagetgtagatcttagccactttttaaaagaaaagggggga
ctggaagggctaattcactcccaacgaaaataagatctgctttttgcttg
tactgggtctctctggttagaccagatctgagcctgggagctctctggct
aactagggaacccactgcttaagcctcaataaagcttgccttgagtgctt
caagtagtgtgtgcccgtctgttgtgtgactctggtaactagagatccct
cagacccttttagtcagtgtggaaaatctctagcagtagtagttcatgtc
atcttattattcagtatttataacttgcaaagaaatgaatatcagagagt
gagaggaacttgtttattgcagcttataatggttacaaataaagcaatag
catcacaaatttcacaaataaagcatttttttcactgcattctagttgtg
gtttgtccaaactcatcaatgtatcttatcatgtctggctctagctatcc
cgcccctaactccgcccagttccgcccattctccgccccatggctgacta
attttttttatttatgcagaggccgaggccgcctcggcctctgagctatt
ccagaagtagtgaggaggcttttttggaggcctagacttttgcagagacg
gcccaaattcgtaatcatggtcatagctgtttcctgtgtgaaattgttat
ccgctcacaattccacacaacatacgagccggaagcataaagtgtaaagc
ctggggtgcctaatgagtgagctaactcacattaattgcgttgcgctcac
tgcccgctttccagtcgggaaacctgtcgtgccagctgcattaatgaatc
ggccaacgcgcggggagaggcggtttgcgtattgggcgctcttccgcttc
ctcgctcactgactcgctgcgctcggtcgttcggctgcggcgagcggtat
cagctcactcaaaggcggtaatacggttatccacagaatcaggggataac
gcaggaaagaacatgtgagcaaaaggccagcaaaaggccaggaaccgtaa
aaaggccgcgttgctggcgtttttccataggctccgcccccctgacgagc
atcacaaaaatcgacgctcaagtcagaggtggcgaaacccgacaggacta
taaagataccaggcgtttccccctggaagctccctcgtgcgctctcctgt
tccgaccctgccgcttaccggatacctgtccgcctttctcccttcgggaa
gcgtggcgctttctcatagctcacgctgtaggtatctcagttcggtgtag
gtcgttcgctccaagctgggctgtgtgcacgaaccccccgttcagcccga
ccgctgcgccttatccggtaactatcgtcttgagtccaacccggtaagac
acgacttatcgccactggcagcagccactggtaacaggattagcagagcg
aggtatgtaggcggtgctacagagttcttgaagtggtggcctaactacgg
ctacactagaaggacagtatttggtatctgcgctctgctgaagccagtta
ccttcggaaaaagagttggtagctcttgatccggcaaacaaaccaccgct
ggtagcggtggtttttttgtttgcaagcagcagattacgcgcagaaaaaa
aggatctcaagaagatcctttgatcttttctacggggtctgacgctcagt
ggaacgaaaactcacgttaagggattttggtcatgagattatcaaaaagg
atcttcacctagatccttttaaattaaaaatgaagttttaaatcaatcta
aagtatatatgagtaaacttggtctgacagttaccaatgcttaatcagtg
aggcacctatctcagcgatctgtctatttcgttcatccatagttgcctga
ctccccgtcgtgtagataactacgatacgggagggcttaccatctggccc
cagtgctgcaatgataccgcgagacccacgctcaccggctccagatttat
cagcaataaaccagccagccggaagggccgagcgcagaagtggtcctgca
actttatccgcctccatccagtctattaattgttgccgggaagctagagt
aagtagttcgccagttaatagtttgcgcaacgttgttgccattgctacag
gcatcgtggtgtcacgctcgtcgtttggtatggcttcattcagctccggt
tcccaacgatcaaggcgagttacatgatcccccatgttgtgcaaaaaagc
ggttagctccttcggtcctccgatcgttgtcagaagtaagttggccgcag
tgttatcactcatggttatggcagcactgcataattctcttactgtcatg
ccatccgtaagatgcttttctgtgactggtgagtactcaaccaagtcatt
ctgagaatagtgtatgcggcgaccgagttgctcttgcccggcgtcaatac
gggataataccgcgccacatagcagaactttaaaagtgctcatcattgga
aaacgttcttcggggcgaaaactctcaaggatcttaccgctgttgagatc
cagttcgatgtaacccactcgtgcacccaactgatcttcagcatctttta
ctttcaccagcgtttctgggtgagcaaaaacaggaaggcaaaatgccgca
aaaaagggaataagggcgacacggaaatgttgaatactcatactcttcct
ttttcaatattattgaagcatttatcagggttattgtctcatgagcggat
acatatttgaatgtatttagaaaaataaacaaataggggttccgcgcaca
tttccccgaaaagtgccacctgacgtctaagaaaccattattatcatgac
attaacctataaaaataggcgtatcacgaggccctttcgtctcgcgcgtt
tcggtgatgacggtgaaaacctctgacacatgcagctcccggagacggtc
acagcttgtctgtaagcggatgccgggagcagacaagcccgtcagggcgc
gtcagcgggtgttggcgggtgtcggggctggcttaactatgcggcatcag
agcagattgtactgagagtgcaccatatgcggtgtgaaataccgcacaga
tgcgtaaggagaaaataccgcatcaggcgccattcgccattcaggctgcg
caactgttgggaagggcgatcggtgcgggcctcttcgctattacgccagc
tggcgaaagggggatgtgctgcaaggcgattaagttgggtaacgccaggg
ttttcccagtcacgacgttgtaaaacgacggccagtgccaagctg
[SEQ ID No: 82]

Preferably, the genetic construct comprises a nucleic acid sequence substantially as set out in SEQ ID No: 82, or a fragment or variant thereof.

The invention also extends to recombinant vectors comprising the genetic construct, as vehicles for therapeutic delivery.

Accordingly, in a second aspect, there is provided a recombinant vector comprising the genetic construct according to the first aspect.

The vector comprising the genetic construct of the first aspect may for example be a plasmid, cosmid or phage and/or a viral vector. Such recombinant vectors are highly useful in the delivery systems of the invention for transforming cells with the nucleotide sequences. Preferably, the vector is a viral vector.

The viral vector may be selected from an adenovirus vector, adeno-associated virus (AAV) vector, retrovirus vector, or a lentivirus vector.

The vector may be a recombinant adeno-associated virus (rAAV) vector. The rAAV may be a naturally occurring vector or a vector with a hybrid AAV serotype. The rAAV may be AAV-1, AAV-2, AAV-3A, AAV-3B, AAV-4, AAV-5, AAV-6, AAV-7, AAV-8, AAV-9, AAV-10, and AAV-11. AAV-2 is most preferred.

The term “recombinant AAV (rAAV) vector” as used herein can mean a recombinant AAV-derived nucleic acid containing at least one terminal repeat sequence.

Preferably, however, the vector is a recombinant lentiviral vector. The lentiviral vector may be integrative or non-integrative. Preferably, the lentiviral vector is non-integrative.

Preferably, the vector of the second aspect is an episomal vector.

Preferably, the vector of the second aspect is recombinant. Recombinant vectors may also include other functional elements. For example, they may further comprise a variety of other functional elements. For instance, the vector is capable of autonomously replicating in the nucleus of the host cell or the vector is preferably incapable of autonomously replicating in the nucleus of the host cell. In this case, elements which induce or regulate DNA replication may be required in the recombinant vector. Alternatively, the recombinant vector may be designed such that it integrates into the genome of a host cell. In this case, DNA sequences which favour targeted integration (e.g. by homologous recombination) are envisaged. Suitable promoters may include the SV40 promoter, CMV, EF1a, PGK, viral long terminal repeats, as well as inducible promoters, such as the Tetracycline inducible system, as examples. The cassette or vector may also comprise a terminator, such as the Beta globin, SV40 polyadenylation sequences or synthetic polyadenylation sequences. The recombinant vector may also comprise a regulator or enhancer to control expression of the nucleic acid as required.

The vector may also comprise DNA coding for a gene that may be used as a selectable marker in the cloning process, i.e. to enable selection of cells that have been transfected or transformed, and to enable the selection of cells harbouring vectors incorporating heterologous DNA. For example, ampicillin, neomycin, puromycin or chloramphenicol resistance is envisaged. Alternatively, the selectable marker gene may be in a different vector to be used simultaneously with the vector containing the genetic construct. The vector may also comprise DNA involved with regulating expression of the nucleotide sequence, or for targeting the expressed polypeptide to a certain part of the host cell.

In a third aspect, there is provided the genetic construct according to the first aspect, or the vector according to the second aspect, for use in therapy.

In a fourth aspect, there is provided the genetic construct according to the first aspect, or the vector according to the second aspect, for use in treating, preventing or ameliorating cancer.

In a fifth aspect, there is provided a method of treating, preventing or ameliorating cancer in a subject, the method comprising administering, or having administered, to a patient in need of such treatment, a therapeutically effective amount of the genetic construct according to the first aspect, or the vector according to the second aspect.

Preferably, the cancer is T-ALL.

It will be appreciated that the genetic construct or the vector according to the invention (collectively referred to herein as “agents”) may be used in a monotherapy (e.g. the use of the genetic constructor the vector alone), for therapy, preferably for treating, ameliorating or preventing cancer. Alternatively, agents according to the invention may be used as an adjunct to, or in combination with, known therapies for therapy, preferably for treating, ameliorating, or preventing cancer.

The agents according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given.

Medicaments comprising agents of the invention may be used in a number of ways. For instance, oral administration may be required, in which case the agents may be contained within a composition that may, for example, be ingested orally in the form of a tablet, capsule or liquid. Compositions comprising agents and medicaments of the invention may be administered by inhalation (e.g. intranasally). Compositions may also be formulated for topical use. For instance, creams or ointments may be applied to the skin.

Agents and medicaments according to the invention may also be incorporated within a slow-or delayed-release device. Such devices may, for example, be inserted on or under the skin, and the medicament may be released over weeks or even months. The device may be located at least adjacent the treatment site. Such devices may be particularly advantageous when long-term treatment with agents used according to the invention is required and which would normally require frequent administration (e.g. at least daily injection).

In a preferred embodiment, agents and medicaments according to the invention may be administered to a subject by injection into the blood stream or directly into a site requiring treatment. Injections may be intravenous (bolus or infusion) or subcutaneous (bolus or infusion), or intradermal (bolus or infusion).

It will be appreciated that the amount of the genetic construct or the vector (i.e. agent) that is required is determined by its biological activity and bioavailability, which in turn depends on the mode of administration, the physiochemical properties of the agent, and whether it is being used as a monotherapy or in a combined therapy. The frequency of administration will also be influenced by the half-life of the agent within the subject being treated. Optimal dosages to be administered may be determined by those skilled in the art, and will vary with the particular agent in use, the strength of the pharmaceutical composition, the mode of administration, and the advancement of the disease being treated, for example cancer. Additional factors depending on the particular subject being treated will result in a need to adjust dosages, including subject age, weight, gender, diet, and time of administration.

Generally, a daily dose of between 0.001 µg/kg of body weight and 10 mg/kg of body weight of agent according to the invention may be used for therapy, and in particular for treating, ameliorating, or preventing cancer, depending upon which agent. More preferably, the daily dose of agent is between 0.01 µg/kg of body weight and 1 mg/kg of body weight, more preferably between 0.1 µg/kg and 100 µg/kg body weight, and most preferably between approximately 0.1 µg/kg and 10 µg/kg body weight.

Alternatively, the dose administered to a subject may be between 0.5×10⁷ and 5 ×10¹² Transducing Units (TU)/Kg of body weight. More preferably, the dose administered to a subject may be between 0.5×10⁸ to 5 ×10¹¹ TU/Kg of body weight. Most preferably, the dose administered to a subject may be between 0.5×10⁹ to 5 ×10¹⁰ TU/Kg of body weight.

The agent may be administered before, during or after onset of the cancer. Daily doses may be given as a single administration (e.g. a single daily injection). Alternatively, the agent may require administration twice or more times during a day. As an example, agents may be administered as two (or more depending upon the severity of the disease being treated, for example cancer) daily doses of between 0.07 µg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, the agent may require administration once a week for even once a month. Alternatively, a slow release device may be used to provide optimal doses of agents according to the invention to a patient without the need to administer repeated doses. Known procedures, such as those conventionally employed by the pharmaceutical industry (e.g. in vivo experimentation, clinical trials, etc.), may be used to form specific formulations of the agents according to the invention and precise therapeutic regimes (such as daily doses of the agents and the frequency of administration).

In a sixth aspect of the invention, there is provided a pharmaceutical composition comprising the genetic construct according to the first aspect, or the vector according to the second aspect, and optionally a pharmaceutically acceptable vehicle.

The pharmaceutical composition is preferably an anti-cancer composition, i.e. a pharmaceutical formulation used in the therapeutic amelioration, prevention or treatment of cancer in a subject, and preferably used in the therapeutic amelioration, prevention or treatment of T-ALL in a subject.

The invention also provides in a seventh aspect, a process for making the pharmaceutical composition according to the sixth aspect, the process comprising combining a therapeutically effective amount of the genetic construct according to the first aspect, or the vector according to the second aspect, with a pharmaceutically acceptable vehicle.

A “subject” may be a vertebrate, mammal, or domestic animal. Hence, medicaments according to the invention may be used to treat any mammal, for example livestock (e.g. a horse), pets, or may be used in other veterinary applications. Most preferably, the subject is a human being.

A “therapeutically effective amount” of the genetic construct or the vector is any amount which, when administered to a subject, is the amount of agent that is needed to treat the disease being treated, for example cancer, or produce the desired effect.

For example, the therapeutically effective amount of the genetic construct or the vector used may be from about 0.001 ng to about 1 mg, and preferably from about 0.01 ng to about 100 ng. It is preferred that the amount the genetic construct or the vector is an amount from about 0.1 ng to about 10 ng, and most preferably from about 0.5 ng to about 5 ng.

A “pharmaceutically acceptable vehicle” as referred to herein, is any known compound or combination of known compounds that are known to those skilled in the art to be useful in formulating pharmaceutical compositions.

In one embodiment, the pharmaceutically acceptable vehicle may be a solid, and the composition may be in the form of a powder or tablet. A solid pharmaceutically acceptable vehicle may include one or more substances which may also act as flavouring agents, lubricants, solubilisers, suspending agents, dyes, fillers, glidants, compression aids, inert binders, sweeteners, preservatives, dyes, coatings, or tablet-disintegrating agents. The vehicle may also be an encapsulating material. In powders, the vehicle is a finely divided solid that is in admixture with the finely divided active agents according to the invention. In tablets, the active agent may be mixed with a vehicle having the necessary compression properties in suitable proportions and compacted in the shape and size desired. The powders and tablets preferably contain up to 99% of the active agents. Suitable solid vehicles include, for example calcium phosphate, magnesium stearate, talc, sugars, lactose, dextrin, starch, gelatin, cellulose, polyvinylpyrrolidine, low melting waxes and ion exchange resins. In another embodiment, the pharmaceutical vehicle may be a gel and the composition may be in the form of a cream or the like.

However, the pharmaceutical vehicle may be a liquid, and the pharmaceutical composition is in the form of a solution. Liquid vehicles are used in preparing solutions, suspensions, emulsions, syrups, elixirs and pressurized compositions. The active agent according to the invention may be dissolved or suspended in a pharmaceutically acceptable liquid vehicle such as water, an organic solvent, a mixture of both or pharmaceutically acceptable oils or fats. The liquid vehicle can contain other suitable pharmaceutical additives such as solubilisers, emulsifiers, buffers, preservatives, sweeteners, flavouring agents, suspending agents, thickening agents, colours, viscosity regulators, stabilizers or osmo-regulators. Suitable examples of liquid vehicles for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can also be an oily ester such as ethyl oleate and isopropyl myristate. Sterile liquid vehicles are useful in sterile liquid form compositions for parenteral administration. The liquid vehicle for pressurized compositions can be a halogenated hydrocarbon or other pharmaceutically acceptable propellant.

Liquid pharmaceutical compositions, which are sterile solutions or suspensions, can be utilized by, for example, intramuscular, intrathecal, epidural, intraperitoneal, intravenous and particularly subcutaneous injection. The agent may be prepared as a sterile solid composition that may be dissolved or suspended at the time of administration using sterile water, saline, or other appropriate sterile injectable medium.

The agents and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The agents used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

As discussed herein, the genetic construct of the invention may comprise a reporter gene, which may be specifically expressed in diseased cells. Thus, advantageously, the genetic construct and vector of the invention may also be used as a robust diagnostic tool. The construct of the invention can advantageously be used to detect the miRNA expression profile of a tumour in a patient, identifying the tumour subtype, which can be used to determine a suitable treatment for the patient. Alternatively, the construct of the invention can be used to simply detect the presence of a cancer.

Accordingly, in an eighth aspect, there is provided the genetic construct according to the first aspect, or the vector according to the second aspect, for use in diagnosis.

In a ninth aspect, there is provided the genetic construct according to the first aspect, or the vector according to the second aspect, for use in diagnosing cancer.

In a tenth aspect, there is provided a method of diagnosis or prognosis, the method comprising detecting the reporter molecule of the genetic construct according to the first aspect, or the vector according to the second aspect in the sample obtained from a subject.

In an eleventh aspect, there is provided a method of diagnosing or prognosing cancer in a subject, the method comprising detecting the reporter molecule of the genetic construct according to the first aspect, or the vector according to the second aspect in a sample obtained from a subject.

Preferably, the cancer is T-ALL.

Prognosis may relate to determining the therapeutic outcome in a subject that has been diagnosed with a disease, preferably cancer. Prognosis may relate to predicting the rate of progression or improvement and/or the duration of disease in a subject, the probability of survival, and/or the efficacy of various treatment regimes. Thus, a poor prognosis may be indicative of disease progression, low probability of survival and reduced efficacy of a treatment regime. A favourable prognosis may be indicative of disease resolution, high probability of survival and increased efficacy of a treatment regime.

The diagnostic method may be performed in vivo. However, the method of diagnosis is preferably performed in-vitro or ex-vivo. Preferably the method of diagnosis is performed in-vitro. Preferably the method of diagnosis is performed ex-vivo.

The invention also provides a kit for diagnosing patients suffering from cancer.

In a twelfth aspect, there is provided a kit for diagnosing a subject suffering from cancer, or a pre-disposition thereto, or for providing a prognosis of the subject’s condition, the kit comprising the genetic construct according to the first aspect, or the vector according to the second aspect.

The sample may be a biological sample or an imaging sample.

The imaging sample may be a PET scan image or an MRI image.

Preferably, the sample comprises a biological sample. The sample may be any material that is obtainable from a subject.

The biological sample may be tissue or a biological fluid. Furthermore, the sample may be blood, plasma, serum, spinal fluid, urine, sweat, saliva, tears, breast aspirate, breast milk, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, lymph, granuloma, cerebrospinal fluid, cancer biopsy or combinations thereof.

The sample may be a liquid aspirate. For example, the sample may be bronchial alveolar lavage (BAL), ascites, pleural lavage, or pericardial lavage.

The sample may comprise blood, urine, tissue etc. In one preferred embodiment, the biological sample comprises a blood sample. The blood may be venous or arterial blood.

Blood samples may be assayed immediately. Alternatively, the blood sample may be stored at low temperatures, for example in a fridge or even frozen before the method is conducted. Alternatively, the blood sample may be stored at room temperature, for example between 18 to 22° C., before the method is conducted. The blood sample may comprise blood serum. The blood sample may comprise blood plasma. Preferably, however, the detection is carried out on whole blood and most preferably the blood sample is peripheral blood.

The blood sample may comprise circulating tumour cells, such that the construct can be used to detect the miRNA expression profile of the circulating tumour cells and thus identify the best treatment option for the patient.

The blood may be further processed before the diagnostic method is performed. For instance, an anticoagulant, such as citrate (such as sodium citrate), hirudin, heparin, PPACK, or sodium fluoride may be added. Thus, the sample collection container may contain an anticoagulant in order to prevent the blood sample from clotting.

In one embodiment, the construct of the invention may be used to detect cancer cells, or to determine cancer subtype, in the cerebrospinal fluid that has been obtained from a patient. Thus, preferably, the biological sample may comprise cerebrospinal fluid

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-82 and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:- (i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty = 15.0, Gap Extension Penalty = 6.66, and Matrix = Identity. For protein alignments: Gap Open Penalty = 10.0, Gap Extension Penalty = 0.2, and Matrix = Gonnet. For DNA and Protein alignments: ENDGAP = -1, and GAPDIST = 4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:- Sequence Identity = (N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3x sodium chloride/sodium citrate (SSC) at approximately 45° C. followed by at least one wash in 0.2x SSC/0.1% SDS at approximately 20-65° C. Alternatively, a substantially similar polypeptide may differ by at least 1, but less than 5, 10, 20, 50 or 100 amino acids from the sequences shown in, for example, in those of SEQ ID Nos: 1 to 82 that are amino acid sequences.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example, small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:-

FIG. 1 shows a schematic representation of miRNA-mediated silencing of a target mRNA. Both mRNAs and miRNAs are transcribed from their gene precursors. The miRNAs then bind to complementary regions of their target mRNAs leading to their degradation.

FIG. 2 shows a schematic representation of one embodiment of the microRNA-detector system and associated genetic construct of the invention for therapeutic gene delivery in T-ALL cells.

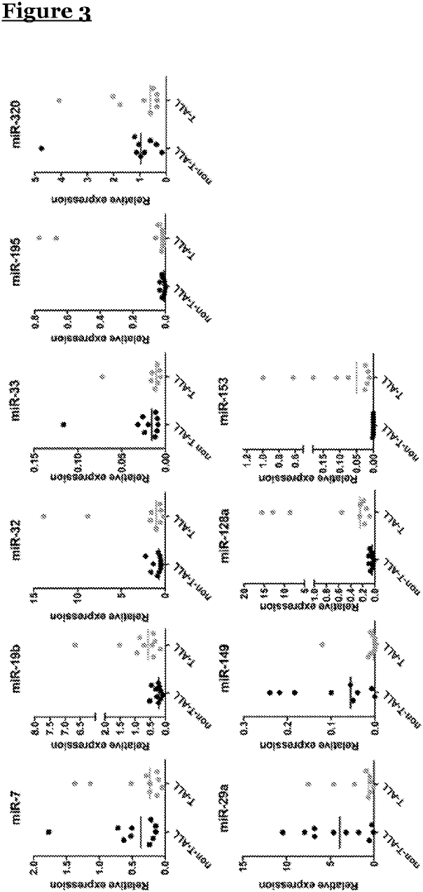

FIG. 3 shows microRNA relative expression in T-ALL and non-T-ALL cells determined by RT-qPCR and calculated using the 2^{-ΔΔCt} method. * p<0.05; *** p<0.0001.

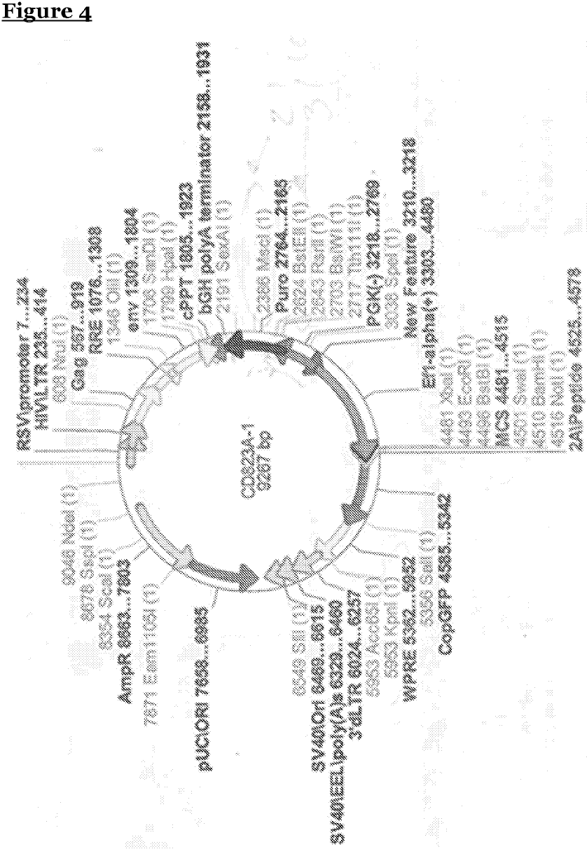

FIG. 4 shows a schematic representation of one embodiment of a pCDH-EF1-MCS-T2A-GFP (PGK-Puro) expression vector from SBI.

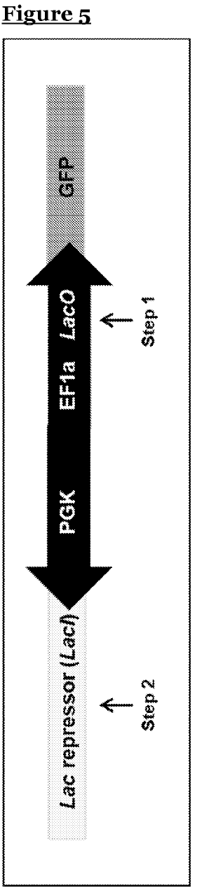

FIG. 5 shows a schematic representation of a bidirectional lentiviral expression vector (“bdLV”) modified to express the Lac operon depicting a 2-step approach.

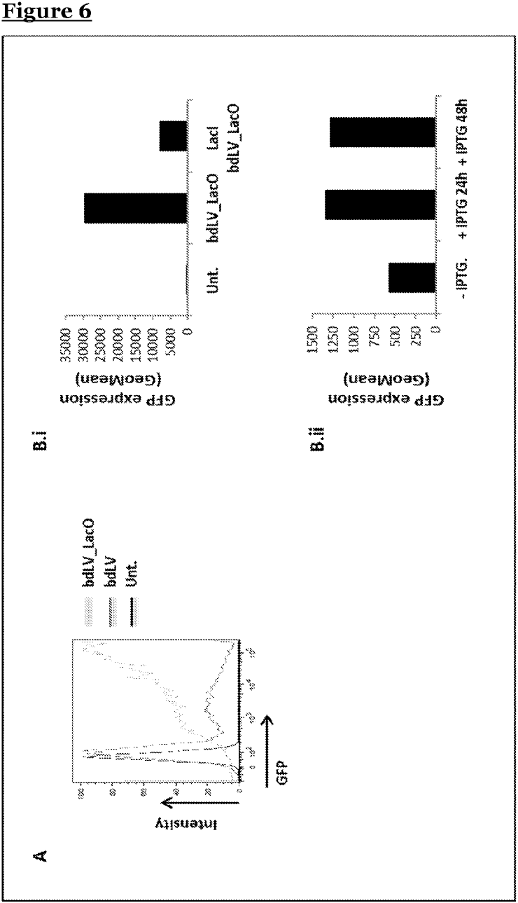

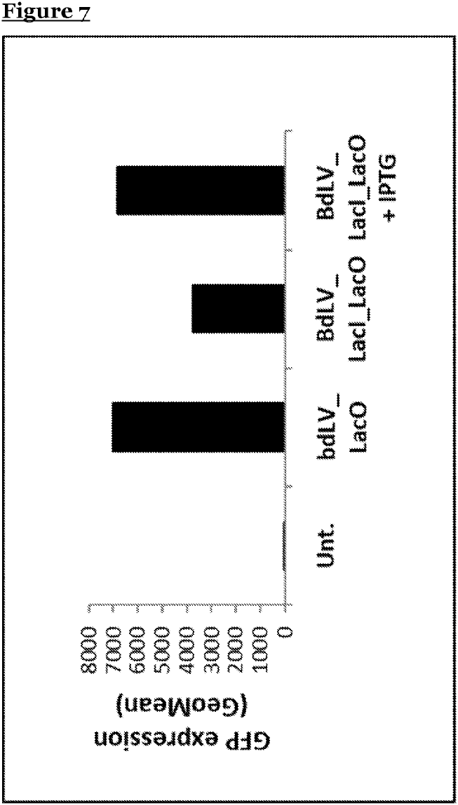

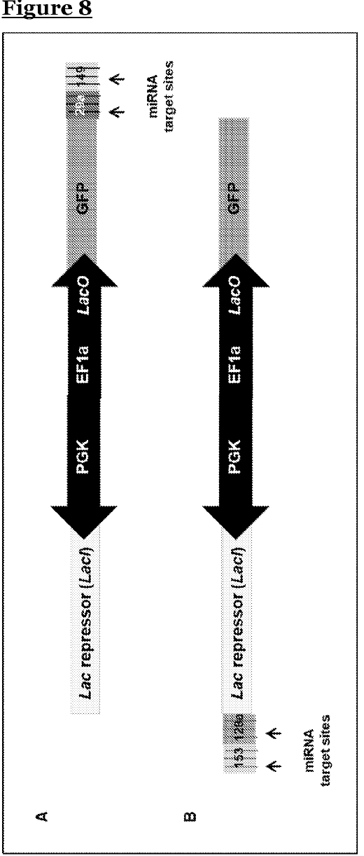

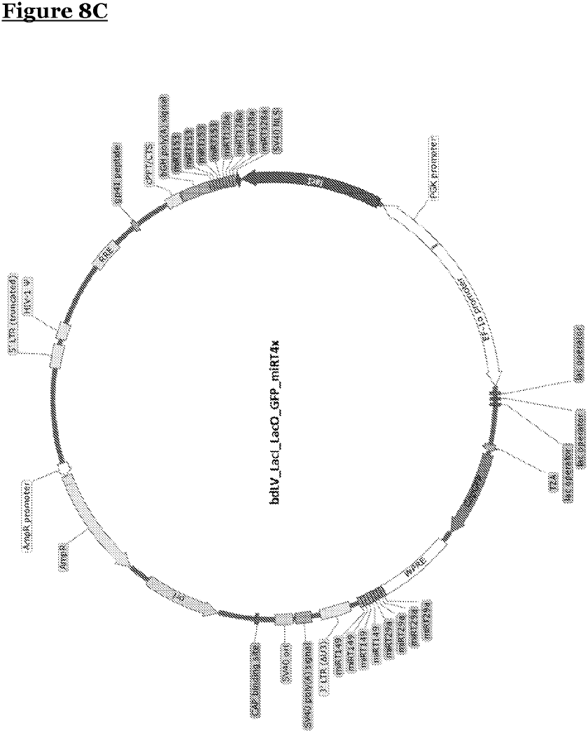

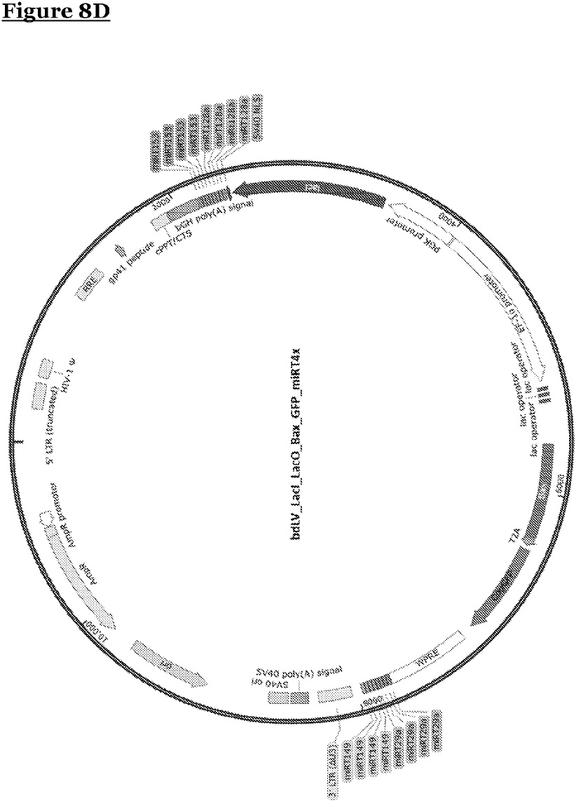

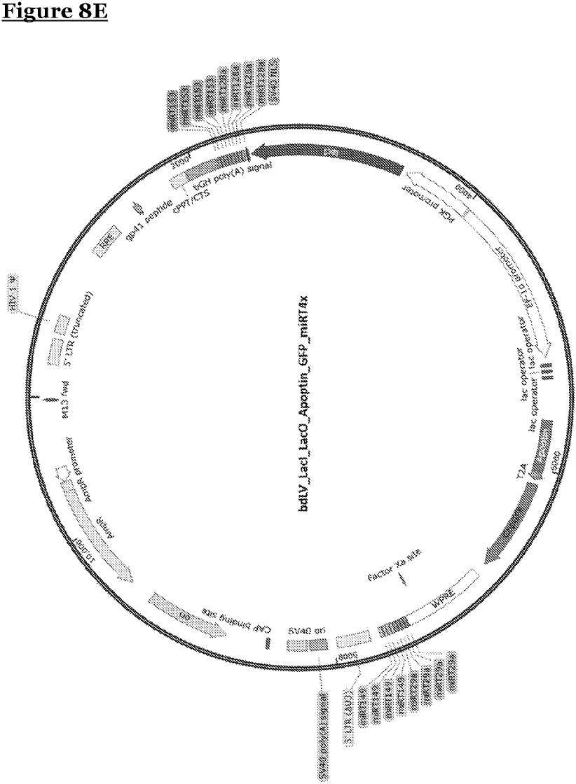

FIG. 6 shows the results from cloning of Lac operator sequences (LacO) downstream of the EF1 promoter. A) shows GFP expression of 293T cells transfected with bdLV vector or bdLV_LacO vector. B.i) shows GFP expression of 293T cells transfected with bdLV_LacO vector or BdLV_LacO and LacI vectors. B.ii) shows GFP expression of 293T cells co-transfected with bdLV_LacO and LacI vectors and treated or not with IPTG (Isopropyl β-D-1-thiogalactopyranoside) for 24 and 48h. Unt.-untransfected.

FIG. 7 shows the results from cloning of the Lac repressor (LacI) downstream of PGK promoter. GFP expression of 293T cells transfected with bdLV_LacO vector or bdLV_LacI_LacO vector and treated with IPTG (Intensity and GeoMean). Unt.-untransfected.

FIG. 8 shows schematic representations of two embodiments of the microRNA detector system (A and B) and associated genetic constructs with the possible combinations of microRNA target sites C, D, E, F G, H, I, J, K and L).

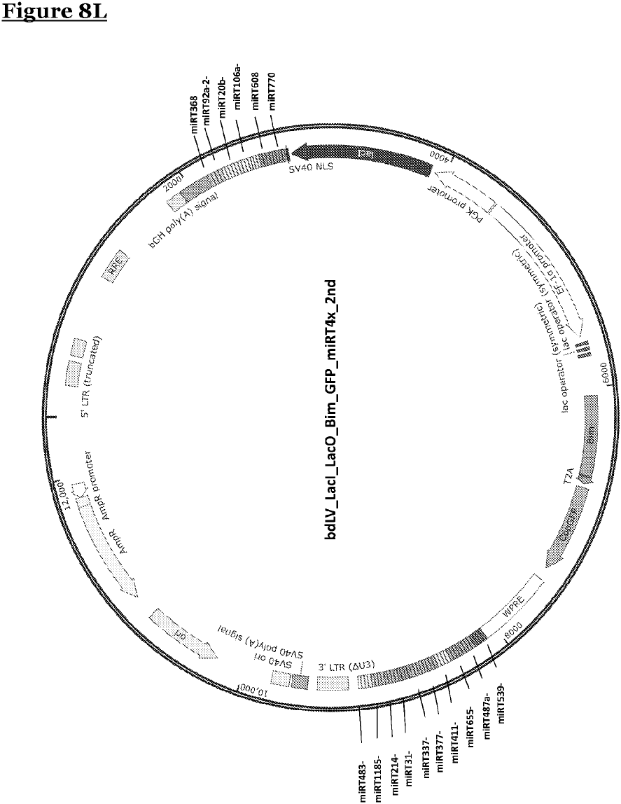

FIGS. 9A-9B show the in vitro validation of the efficiency and specificity of the microRNA-detectors with target sequences for miR-29a and miR-149. T-ALL (A) and non-T-ALL (B) cells were transduced with the microRNA-detectors BdLV_miR29a_4xT, BdLV_miR149_4xT, and BdLV_miR29a_4xT_miR149_4xT. GFP expression was analysed by flow cytometry and used as readout to determine the efficiency of the detectors to repress the reporter gene expression.

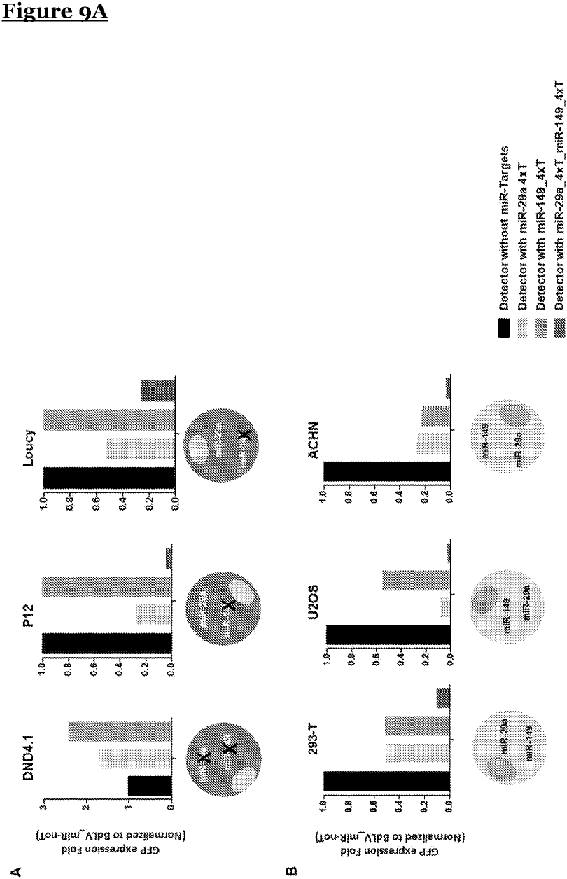

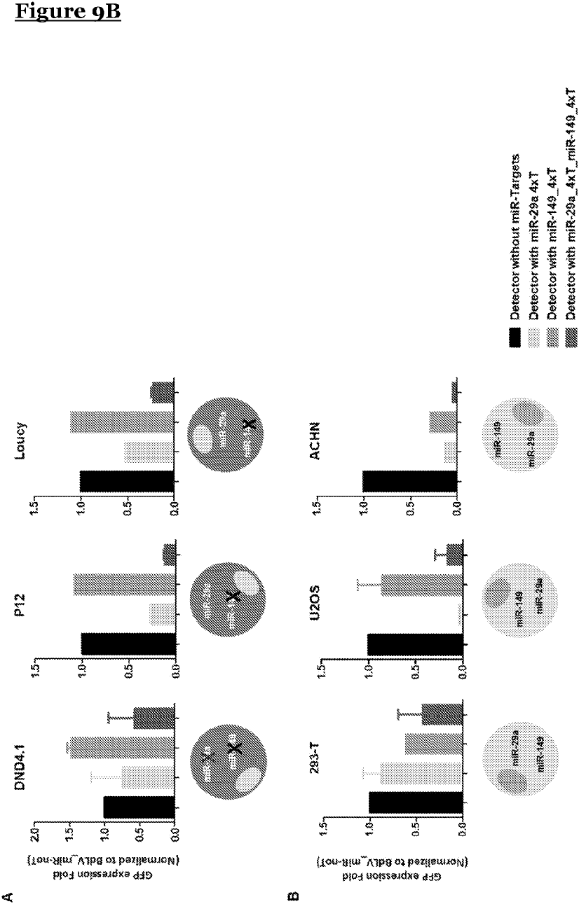

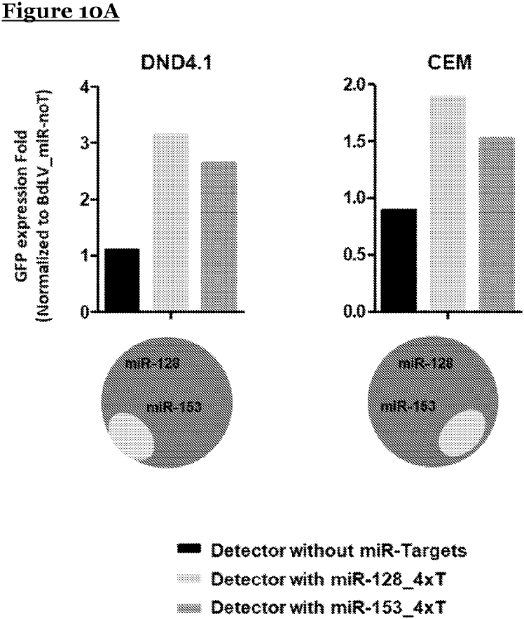

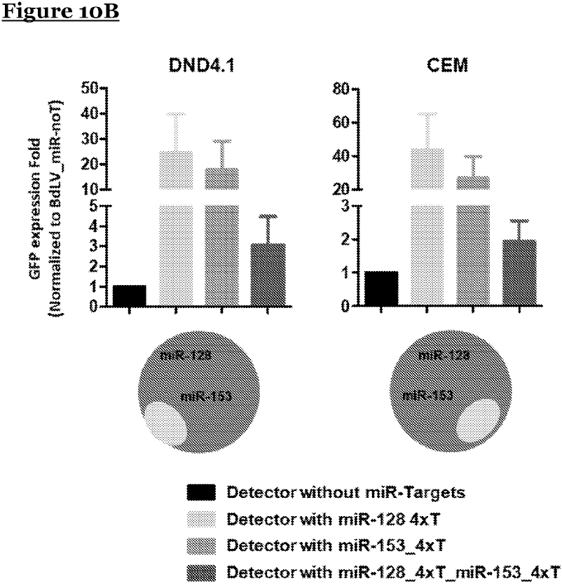

FIGS. 10A-10B show the in vitro validation of the efficiency and specificity of the microRNA-detectors with target sequences for miR-128a and miR-153. T-ALL cells were transduced with the microRNA-detectors BdLV_miR128a_4xT, BdLV_miR153_4xT and BdLV_miR128a_4xT_miR153_4xT. GFP expression was analyzed by flow cytometry and used as readout to determine the efficiency of the microRNA-detectors to de-repress the reporter gene promoter through LacI.

FIG. 11 shows in vitro validation of a construct comprising dual miRNA target sites.

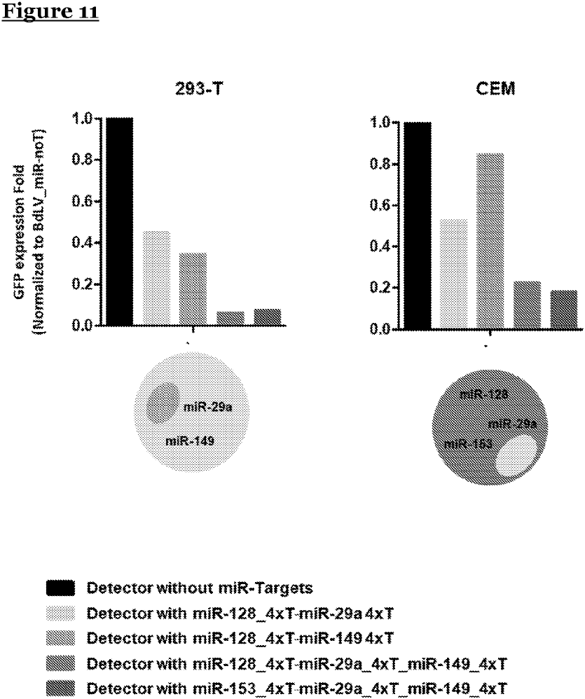

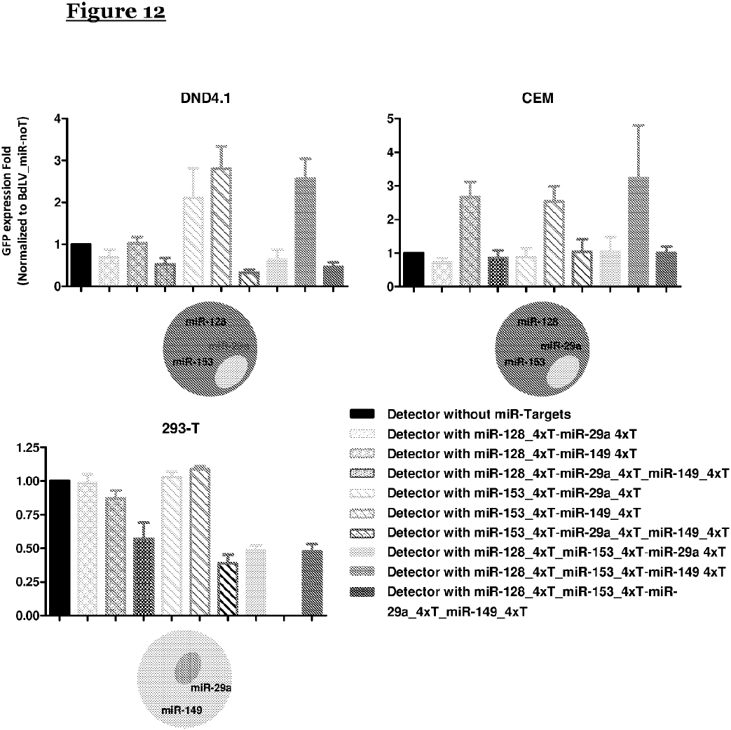

FIG. 12 shows the in vitro validation of the efficiency and specificity of the micro-RNA detectors comprising two, three or four miRNA target sites.

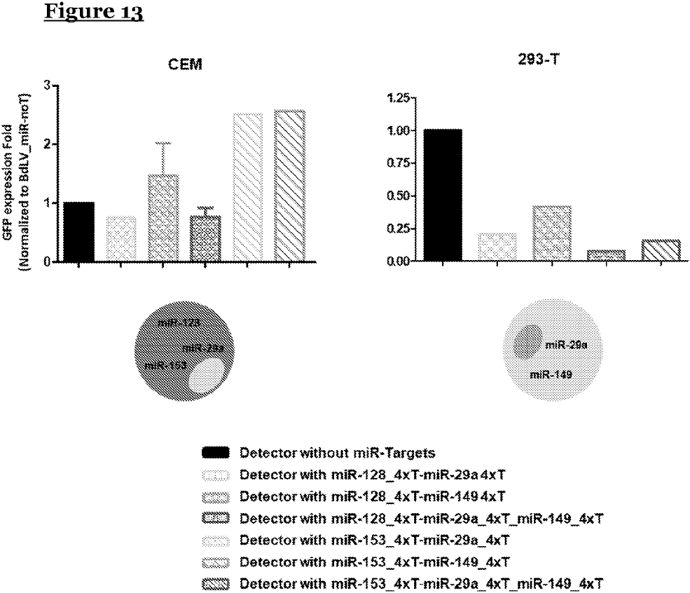

FIG. 13 shows the in vitro validation of the efficiency and specificity of the micro-RNA detectors in cultures of T-ALL and non-T-ALL cells transduced with non-integrative lentiviral vectors.

### EXAMPLES

### Example 1 - Overview of a Novel MicroRNA-Detector Which Enables T-ALL Identification and Selective Inhibition

In cancer, microRNA expression profiles have been shown to distinguish tumour cells from normal cells, and to discriminate tumours of different developmental origin and their differentiation state (8, 9, 14, 15). Therefore, the inventors took advantage of T-ALL-specific microRNA expression profiles to develop a microRNA-detector system (i.e. the recombinant genetic construct of the invention) that uses microRNAs to: (1) identify T-ALL cells, and then (2) regulate the expression of a therapeutic gene (an apoptosis-inducing gene) into leukaemia cells. Based on a pre-determined microRNA profile that is specific to T-ALL cells only, when expressed inside a host cell, the construct first determines whether the host cells are leukaemia cells and, if so, the construct then induces cell death. Thus, a positive match between the expression of specific microRNAs inside a T-ALL cell and the construct results in the delivery of a therapeutic gene to those T-ALL cells, inducing their death but not of the normal healthy cells (see FIG. 2A). The intrinsic ability of this system to distinguish between a T-ALL cell and a non-T-ALL cell within the body of the patient is, hence, a unique aspect of the invention. This therefore provides the significant advantage over other gene therapy technologies applied to precision oncology, and it has been deliberately designed to provide the best possible efficacy and safety outcomes for patients.

The delivery of the therapeutic gene is regulated both by microRNAs which are present only (or highly expressed) in T-ALL cells as well as microRNAs which are specifically absent (or significantly less expressed) in those cells. For example, referring to FIG. 2B, assuming that a leukaemia cell can be discriminated from normal cells by the absence of the microRNAs A and B, and by the presence of the microRNAs C and D, then, the therapeutic gene will only be delivered if: (1) microRNAs A and B are absent; and (2) microRNAs C and D are expressed in the same cell (see FIG. 2B).

MicroRNAs have been previously explored to negatively regulate gene expression. However, the present invention uses the concomitant presence and absence of specific microRNAs to positively regulate the expression of the therapeutic gene. This dual layer of regulation is achieved by the use of a bidirectional expression vector and a negative feedback loop. Such a system has not been applied before and increases the specificity of the technology to deliver the therapeutic gene only to the target cells, maximizing drug efficacy and safety, and patients’ therapeutic outcome (see FIG. 2C).

This innovative technology has the potential to effectively circumvent the lack of specificity in T-ALL therapy. Importantly, it will serve as proof-of-concept that this strategy can be applied to the development of similar personalized therapies in other cancer types in which there are variances in microRNA expression profiles.

### Materials and Methods

### Bioinformatics Analysis

To define a microRNA expression profile that is specific of T-ALL cells, the inventors collected microRNA expression data from publicly available human datasets. The inventors initially identified 10 human datasets that include T-ALL samples (1-10). Of these, four were excluded for lacking non-tumor samples (1-4) and two others for the impossibility to access the raw data (5, 6). For each one of the datasets analyzed (7-10), the inventors compared the non-T-ALL samples (control group) with the T-ALL samples (T-ALL group). The samples analyzed in the control groups varied between studies and included healthy tissue of lung, colon, bladder, brain, kidney, breast, total bone marrow (BM) cells, total thymocytes, hematopoietic CD34+ BM cells, CD4+ T cells, CD8+ T cells and CD4+CD8+CD3+ T-cells. The T-ALL groups included T-ALL primary cells and cell lines.

To be considered up-regulated in the T-ALL samples, the minimum expression value of a certain miRNA in that group had to be higher than the maximum expression value of that microRNA in the control group. In the same way, when the minimum expression value of a microRNA in the control group was higher than the maximum expression value in the T-ALL group, that miRNA was considered down-regulated in the T-ALL samples. Using this classification system, the inventors were able to identify 12 miRNAs which were down-regulated and up-regulated in the different datasets.

Subsequently, the inventors established a further T-ALL-specific microRNA expression profile by bioinformatics analyses of publicly available microRNA data obtained from human T-ALL cells and normal cells and tissues. These datasets encompassed approximately 140 T-ALL cell lines and patient cells and approximately 480 normal tissue samples including brain, liver, kidney, lung, heart, breast, bladder, colon, uterus, skin, ovary, pancreas, prostate, stomach, testis, meninges, thyroid, thymus, bone marrow, spleen, lymph nodes and peripheral blood. The hematopoietic and lymphoid organs covered different lineages and different differentiation stages, including hematopoietic stem and precursor cells.

This analysis identified 25 microRNAs, as shown in Table 1 below.

TABLE 1

| | | | | |
|---|---|---|---|---|
| Expression in T-ALL | microR NA ID | Accession Number | Sequence | Target sequence |
| up-regulated | hsa-miR-3687 | MIMAT001 8115 | CCCGGACAGGCGUUCGU GCGACGU (SEQ ID No: 67) | ACGTCGCACGAACGCCT GTCCGGG (SEQ ID No: 56) |
| up-regulated | hsa-miR-92a-2-5p | MIMAT 0004508 | GGGUGGGGAUUUGUUG CAUUAC (SEQ ID No: 68) | GTAATGCAACAAATCCC CACCC (SEQ ID No: 57) |
| up-regulated | hsa-miR-20b-3P | MIMAT 0004752 | ACUGUAGUAUGGGCACU UCCAG (SEQ ID No: 69) | CTGGAAGTGCCCATACT ACAGT (SEQ ID No: 58) |
| up-regulated | hsa-miR-6087 | MIMAT002 | UGAGGCGGGGGGGCGAG C (SEQ ID No: 70) | GCTCGCCCCCCCGCCTC A (SEQ ID No: 59) |
| up-regulated | hsa-miR-106a-3p | MIMAT000 4517 | CUGCAAUGUAAGCACUU CUUAC (SEQ ID No: 71) | GTAAGAAGTGCTTACAT TGCAG (SEQ ID No: 60) |
| up-regulated | hsa-miR-7704 | MIMAT003 oo19 | CGGGGUCGGCGGCGACG UG (SEQ ID No: 72) | CACGTCGCCGCCGACCC CG (SEQ ID No: 61) |
| up-regulated | hsa-miR-5701 | MIMAT002 2494 | UUAUUGUCACGUUCUGA UU (SEQ ID No: 73) | AATCAGAACGTGACAAT AA (SEQ ID No: 62) |
| up-regulated | hsa-miR-766-5p | MIMAT002 2714 | AGGAGGAAUUGGUGCUG GUCUU (SEQ ID No: 74) | AAGACCAGCACCAATTC CTCCT (SEQ ID No: 63) |
| up-regulated | hsa-miR-3609 | MIMAT001 7986 | CAAAGUGAUGAGUAAUA CUGGCUG (SEQ ID No: 75) | CAGCCAGTATTACTCAT CACTTTG (SEQ ID No: 64) |
| up-regulated | hsa-miR-3615 | MIMAT001 7994 | UCUCUCGGCUCCUCGCG GCUC (SEQ ID No: 76) | GAGCCGCGAGGAGCCG AGAGA (SEQ ID No: 65) |
| up-regulated | hsa-miR-4746-5p | MIMAT001 9880 | CCGGUCCCAGGAGAACC UGCAGA (SEQ ID No: 77) | TCTGCAGGTTCTCCTGG GACCGG (SEQ ID No: 66) |
| down-regulated | hsa-miR-539-5p | MIMAT0 003163 | GGAGAAAUUAUCCUUGG UGUGU (SEQ ID No: 42) | ACACACCAAGGATAATT TCTCC (SEQ ID No: 28) |
| down-regulated | hsa-miR-487a-3p | MIMAT000 2178 | AAUCAUACAGGGACAUC CAGUU (SEQ ID No: 43) | AACTGGATGTCCCTGTA TGATT (SEQ ID No: 29) |
| down-regulated | hsa-miR-655-3p | MIMAT000 3331 | AUAAUACAUGGUUAACC UCUUU (SEQ ID No: 44) | AAAGAGGTTAACCATGT ATTAT (SEQ ID No: 30) |
| down-regulated | hsa-miR-411-3p | MIMAT0 004813 | UAUGUAACACGGUCCAC UAACC (SEQ ID No: 45) | GGTTAGTGGACCGTGTT ACATA (SEQ ID No: 31) |
| down-regulated | hsa-miR-377-5P | MIMAT000 4689 | AGAGGUUGCCCUUGGUG AAUUC (SEQ ID No: 46) | GAATTCACCAAGGGCAA CCTCT (SEQ ID No: 32) |
| down-regulated | hsa-miR-337-5P | MIMAT000 4695 | GAACGGCUUCAUACAGG AGUU (SEQ ID No: 47) | AACTCCTGTATGAAGCC GTTC (SEQ ID No: 33) |
| down-regulated | hsa-miR-31-3p | MIMAT000 4504 | UGCUAUGCCAACAUAUU GCCAU (SEQ ID No: 48) | ATGGCAATATGTTGGCA TAGCA (SEQ ID No: 34) |
| down-regulated | hsa-miR-214-5P | MIMAT000 4564 | UGCCUGUCUACACUUGC UGUGC (SEQ ID No: 49) | GCACAGCAAGTGTAGAC AGGCA (SEQ ID No: 35) |
| down-regulated | hsa-miR-1185-5p | MIMAT000 5798 | AGAGGAUACCCUUUGUA UGUU (SEQ ID No: 50) | AACATACAAAGGGTATC CTCT (SEQ ID No: 36) |
| down-regulated | hsa-miR-483-5p | MIMAT000 4761 | AAGACGGGAGGAAAGAA GGGAG (SEQ ID No: 51) | CTCCCTTCTTTCCTCCCG TCTT (SEQ ID No: 37) |
| down-regulated | hsa-miR-365a-3p | MIMAT000 9199 | AGGGACUUUUGGGGGCA GAUGUG (SEQ ID No: 52) | CACATCTGCCCCCAAAA GTCCCT (SEQ ID No: 38) |
| down-regulated | hsa-miR-127-3p | MIMAT000 0446 | UCGGAUCCGUCUGAGCU UGGCU (SEQ ID No: 53) | AGCCAAGCTCAGACGGA TCCGA (SEQ ID No: 39) |
| down-regulated | hsa-miR-574-3p | MIMAT000 3239 | CACGCUCAUGCACACACC CACA (SEQ ID No: 54) | TGTGGGTGTGTGCATGA GCGTG (SEQ ID No: 40) |
| down-regulated | hsa-miR-125b-5p | MIMAT000 0423 | UCCCUGAGACCCUAACU UGUGA (SEQ ID No: 55) | TCACAAGTTAGGGTCTC AGGGA (SEQ ID No: 41) |

Based on this list, the inventors then shortlisted 16 microRNAs most likely to be those used in the construction of the microRNA-detector, as shown in Table 2 below.

TABLE 2

| | | | | |
|---|---|---|---|---|
| Expression in T-ALL | microRNA ID | Accession Number | Sequence | Target sequence |
| up-regulated | hsa-miR-3687 | MIMAT0018115 | CCCGGACAGGCGUUCGUG CGACGU (SEQ ID No: 67) | ACGTCGCACGAACGCCTGT CCGGG (SEQ ID No: 56) |
| up-regulated | hsa-miR-92a-2-5p | MIMAT00045o8 | GGGUGGGGAUUUGUUGC AUUAC (SEQ ID No: 68) | GTAATGCAACAAATCCCCA CCC (SEQ ID No: 57) |
| up-regulated | hsa-miR-20b-3p | MIMAT0004752 | ACUGUAGUAUGGGCACU UCCAG (SEQ ID No: 69) | CTGGAAGTGCCCATACTAC AGT (SEQ ID No: 58) |
| up-regulated | hsa-miR-6087 | MIMAT0023712 | UGAGGCGGGGGGGCGAG C (SEQ ID No: 70) | GCTCGCCCCCCCGCCTCA (SEQ ID No: 59) |
| up-regulated | hsa-miR-106a-3p | MIMAT0004517 | CUGCAAUGUAAGCACUUC UUAC (SEQ ID No: 71) | GTAAGAAGTGCTTACATTG CAG (SEQ ID No: 60) |
| up-regulated | hsa-miR-7704 | MIMAT0030019 | CGGGGUCGGCGGCGACGU G (SEQ ID No: 72) | CACGTCGCCGCCGACCCCG (SEQ ID No: 61) |
| down-regulated | hsa-miR-539-5p | MIMAT0003163 | GGAGAAAUUAUCCUUGG UGUGU (SEQ ID No: 42) | ACACACCAAGGATAATTTC TCC (SEQ ID No: 28) |
| down-regulated | hsa-miR-487a-3p | MIMAT0002178 | AAUCAUACAGGGACAUCC AGUU (SEQ ID No: 43) | AACTGGATGTCCCTGTATG ATT (SEQ ID No: 29) |
| down-regulated | hsa-miR-655-3p | MIMAT0003331 | AUAAUACAUGGUUAACCU CUUU (SEQ ID No: 44) | AAAGAGGTTAACCATGTAT TAT (SEQ ID No: 30) |
| down-regulated | hsa-miR-411-3p | MIMAT0004813 | UAUGUAACACGGUCCACU AACC (SEQ ID No: 45) | GGTTAGTGGACCGTGTTAC ATA (SEQ ID No: 31) |
| down-regulated | hsa-miR-377-5p | MlMAT0004689 | AGAGGUUGCCCUUGGUG AAUUC (SEQ ID No: 46) | GAATTCACCAAGGGCAACC TCT (SEQ ID No: 32) |
| down-regulated | hsa-miR-337-5p | MIMAT0004695 | GAACGGCUUCAUACAGGA GUU (SEQ ID No: 47) | AACTCCTGTATGAAGCCGT TC (SEQ ID No: 33) |
| down-regulated | hsa-miR-31-3p | MlMAT0004504 | UGCUAUGCCAACAUAUUG CCAU (SEQ ID No: 48) | ATGGCAATATGTTGGCATA GCA (SEQ ID No: 34) |
| down-regulated | hsa-miR-214-5p | MIMAT0004564 | UGCCUGUCUACACUUGCU GUGC (SEQ ID No: 49) | GCACAGCAAGTGTAGACAG GCA (SEQ ID No: 35) |
| down-regulated | hsa-miR-1185-5p | MIMAT0005798 | AGAGGAUACCCUUUGUA UGUU (SEQ ID No: 50) | AACATACAAAGGGTATCCT CT (SEQ ID No: 36) |
| down-regulated | hsa-miR-483-5p | MIMAT0004761 | AAGACGGGAGGAAAGAAG GGAG (SEQ ID No: 51) | CTCCCTTCTTTCCTCCCGTC TT (SEQ ID No: 37) |

### Validation of MicroRNA Expression Profile

The validation of the T-ALL-specific microRNA expression profile was performed by real-time PCR quantification of each microRNA expression, using TaqMan MicroRNA assays (Applied Biosystems). MicroRNA expression was analyzed in T-ALL cell lines corresponding to different differentiation stages (Jurkat, DND4.1, MOLT4, CEM, SUP-T1, HPB-ALL, TALL-1, P12 and Loucy) and several non-T-ALL cell lines such as 293T, A549, MDA231, CaCO2, U2OS, HCT-116, ACHN, A498 and D458.

T-ALL cell lines were cultured in RPMI-1640 medium with L-glutamine supplemented with 10% of fetal bovine serum (FBS) and 1% penicillin/streptomycin (Pen/Strep). Non-T-ALL cell lines were cultured as follows: 293T, A549, MDA231 and CaCO₂ in Dulbecco’s Modified Eagle’s medium supplemented with 10% FBS and 1% Pen/Strep; U2OS; HCT-116 in McCoy’s 5A medium with 10% FBS and 1% Pen/Strep; ACHN and A498 in Eagle’s Minimum Essential Medium with 10% FBS and 1% Pen/Strep; and D458 in Iscove’s Modified Dulbecco’s Media with 10% FBS and 1% Pen/Strep. All cell lines were kept at 37° C. in a 5% CO₂ environment.

Total RNA was extracted from cells using TRIzol reagent, according to manufactures’ protocol. Next, microRNA expression quantification was performed using the Applied Biosystems TaqMan MicroRNA Reverse Transcription Kit, in combination with TaqMan miRNA Assays. This is a two-step process that starts with the reverse transcription of microRNA to cDNA using a microRNA RT specific primer. This reaction was followed by real-time PCR amplification of microRNAs, using microRNA-specific TaqMan probes. The PCR reactions were performed using Vii7 Real Time PCR system. MicroRNA expression was normalized using RNU6b and microRNA relative expression calculated using the 2^{-ΔΔCt} method.

### Technological Design of MicroRNA-Detectors

MicroRNA-detectors (i.e. the constructs of the invention) were built using the pCDH-EF1-MCS-T2A-GFP (PGK-Puro) expression vector (from SBI) as backbone, as shown in FIG. 4. This bidirectional promoter expression vector allows the expression of GFP with a constitutive EF1 promoter. In negative orientation, the PGK promoter drives the expression of the puromycin marker. For proof-of-concept purposes, GFP was used as a surrogate for the therapeutic gene.

The inventors started by introducing the Lac operon system into the vector backbone shown in FIG. 4, the bidirectional lentiviral expression vector (bdLV). This process involved:

### STEP 1 - Cloning Lac Operator Sequences (LacO) Downstream of the EF1 Promoter

For this purpose, the inventors amplified the SV40 intron containing three copies of the Lac Operator sequence (SV40intron/3LacO) from the pOPI3CAT (LacSwitch II Inducible Mammalian Expression System from Agilent) and cloned it into the bdLV (bdLV_LacO). SV40intron/3LacO was amplified using primers with restriction sites for EcoRI and NotI (Table 3). Upon amplification, both PCR product and BdLV vector were digested with EcoRI and NotI restriction enzymes (Thermo Scientific). The digested SV40intron/3LacO PCR product and the BdLV vector were then ligated using T4 DNA ligase (Fermentas). The correct insertion of LacO sequences was confirmed by restriction enzyme diagnostic digestion and Sanger sequencing.

### STEP 2 - Cloning the Lac Repressor (LacI) Downstream of PGK Promoter

This step involved the replacement of the puromycin by LacI (in reverse orientation). The LacI was PCR-amplified from the pCMVLacI vector (LacSwitch II Inducible Mammalian Expression System from Agilent) and sub-cloned into a smaller vector (pcDNA3.1+) to be obtained in reverse orientation. To that end LacI_NLS was amplified using primers with restriction sites for the blunt-end SmaI (Table 3). Upon purification, LacI_NLS PCR product and pcDNA3.1+ vector were digested with SmaI (from NEB). The digested LacI_NLS fragment and pcDNA3.1+ vector were then ligated using T4 DNA ligase (Fermentas). The insertion of LacI in the correct orientation was confirmed by restriction enzyme digestion and Sanger sequencing. Next, directed mutagenesis (QuickChange Lightning Site-Directed Mutagenesis kit from Agilent) was used to create the restriction enzymes sites necessary to cut out the puromycin cDNA from the bdLV_LacO vector and replace it by LacI. Because of bdLV_LacO size (approximately 10 Kb) the region of the bdLV_LacO vector to be mutated (puromycin and flanking sequences) was removed and sub-cloned into pcDNA3.1+ vector. More specifically, the restriction enzymes NotI and EcoRI were used to digest both the bdLV_LacO and pcDNA3.1+ vectors. Puromycin digestion product and digested pcDNA3.1+ were ligated using T4 DNA ligase (Fermentas). This intermediate smaller vector (approximately 6.7 Kb) was used to eliminate one XhoI restriction site and to generate an AvrII restriction site. Once mutated, puromycin and flanking regions was amplified using primers with restriction enzymes sites for HpaI and SpeI (Table 3) and cloned back into bdLV vector upon ligation of the PCR product and vector digested with those enzymes. Puromycin was then removed from bdLV_LacO vector using XhoI and AvrII restriction enzymes while, the same enzymes were used to cut out LacI from pcDNA3.1+ vector_LacI. T4 DNA ligase was used to ligate bdLV_LacO vector and LacI, creating bdLV_LacI_LacO vector. The correct insertion of LacI in the bdLV_LacO vector was confirmed by restriction enzyme diagnostic digestion and Sanger sequencing.

Next, the inventors cloned four target sites of miR-29a, miR-149, or miR-29a and miR-149 (microRNAs specifically absent or down-regulated in T-ALL cells) downstream of the reporter gene promoter, in the bdLV_LacI_LacOconstruct. To this end, they created a restriction enzyme site for XmaI downstream of GFP. Four target sites of each microRNA (or both) were synthesized in tandem and introduced into bdLV-LacI_LacO vector upon digestion with XmaI and ligation with T4 DNA ligase.

In a second phase, the inventors cloned four target sites of miR-128a or miR-153 (microRNAs specifically up-regulated in T-ALL cells) downstream of the repressor of the reporter gene promoter, in the bdLV_LacI_LacO construct. To this end, four target sites of each microRNA were synthesized in tandem and introduced in the bdLV-LacI_LacO vector upon digestion with the restriction enzyme XhoI and ligation with the T4 DNA ligase.

References for the methods used
1) Landgraf, P. et al. (2007) Cell 29;129(7):1401-14
2) Rosenfeld, N. et al. (2008) Nat Biotechnol Apr;26(4):462-9
3) Fulci, V. et al. (2009) Genes, Chromosomes & Cancer 48:1069-1082
4) de Leeuw, D. et al. (2013) Clin Cancer Res; 19(8); 2187-96
5) Schotte, D. et al (2009) Leukemia Feb;23(2):313-22.
6) Coskum, E. et al. (2013) Leukemia Research 37:647- 656
7) Lu, J. et al (2005) Nature. Jun 9;435 (7043):834-8
8) Mavrakis, K.J. et al (2011) Nat Genet. Jun 5;43(7):673-8,
9) Schotte, D. et al. (2011) Haematologica 2011;96(5):703-711
10) Sanghvi, V. R. eta al. (2014) Sci. Signal. Nov 18;7(352):ra111.

TABLE 3

Primer sequences used

| | |
|---|---|
| Primer’s name | Primers sequence |
| EcoR1_LacO | TGA GGC GAA TTC GTA AAT ATA AAA TTT ACT AGG - SEQ ID No: 10 |
| NotI_LacO | TAT AGC GGC CGC CTA AAA TAC ACA AAC A-SEQ ID No: 11 |
| SmaI_LacI F | TAT GCC CGG GGA GGT ACC CTC CCA CCA TG- SEQ ID No: 12 |
| SmaI_LacI R | CCA CCC GGG TCA AAC CTT CCT CTT CTT CTT AGG- SEQ ID No: 13 |
| HpaI_pcDNA3.1 | TGA GCG TTA ACT TTT AAA AGA AAA G- SEQ ID No: 14 |
| SpeI_pcDNA3.1 | TAT AAC TAG TCT CGT GCA GAT GGA CAG CAC CG- SEQ ID No: 15 |

### Example 2 - Definition and Validation of a T-ALL-Specific miRNA Expression Profile

In order to start constructing the microRNA-detector constructs of the invention, the inventors took advantage of publicly available human datasets to establish a provisional T-ALL-specific microRNA expression profile. Using this approach, the inventors were able to identify 12 miRNAs specifically down-regulated and up-regulated in T-ALL cells.

As shown in FIG. 3, the validation of these 12 microRNAs by real-time PCR allowed them to confirm miR-128a and miR-153 as being specifically up-regulated in T-ALL cells, and miR-29a and miR-149 as being specifically down-regulated in T-ALL cells.

Subsequent bioinformatic analyses of a larger cohort of publicly available microRNA data obtained from human T-ALL cells and normal cells and tissues has identified 25 miRNAs as being specifically down-regulated (miR-539-5p, miR-487a-3p, miR-655-3p, miR-411-3p, miR-377-5p, miR-337-5p, miR-31-3p, miR-214-5p, miR-1185-5p, miR-483-5p, miR-365a-3p, miR-127-3p, miR-574-3p and miR-125b-5p) and up-regulated (miR-3687, miR-92a-2-5p, miR-20b-3p, miR-6087, miR-106a-3p, miR-7704, miR-5701, miR-766-5p, miR-3609, miR-3615 and miR-4746-5p) in T-ALL cells in comparison with healthy cells. These have been shortlisted to 16 microRNAs most likely to be used in the construction of the microRNA detector, 10 specifically down-regulated (miR-539-5p, miR-487a-3p, miR-655-3p, miR-411-3p, miR-377-5p, miR-337-5p, miR-31-3p, miR-214-5p, miR-1185-5p and miR-483-5p) and 6 specifically up-regulated (miR-3687, miR-92a-2-5p, miR-20b-3p, miR-6087, miR-106a-3p and miR-7704) in T-ALL cells.

### Example 3 - Design and Construction of microRNA-Detectors/Constructs

The microRNA-detector construct developed by the inventors consists of a bidirectional lentiviral expression vector, in which the therapeutic gene is expressed by one promoter, and a repressor of the therapeutic gene promoter is expressed by the second promoter. The Lac operon system is used as the repressor. The expression of both mRNAs is regulated by T-ALL-cell-specific microRNAs. For proof-of-concept purposes, GFP was used instead of an apoptosis-inducing gene (i.e. GFP expression was used as a surrogate for the therapeutic gene).

As shown in FIG. 4, as the backbone for the detector system, the inventors used the pCDH-EF1-MCS-T2A-GFP (PGK-Puro) expression vector from SBI (referred to as BdLV). This bidirectional promoter expression vector allows the expression of GFP with a constitutive EF1 promoter. In negative orientation the PGK promoter drives the expression of the puromycin marker.

The inventors introduced a Lac operon system in the backbone vector, as shown in FIG. 5, which functioned as the repression system. The process was divided in two steps:

### I) Cloning Lac Operator Sequences (LacO) Downstream of the EF1 Promoter

As shown in FIG. 6, the Lac repressor (LacI) binds to Lac Operator (LacO) regulatory sites in the promoter turning off transcription, confirming that the insertion of LacO sequences does not affect the expression of GFP by the EF1 promoter (FIG. 6A).

The inventors further confirmed that the LacO was functional. The inventors verified that LacI proteins can bind to Lac operator sequences in bdLV_LacO and repress GFP expression by the EF1 promoter. As shown in FIG. 6Bi, transfection of a LacI expression vector in bdLV_LacO expressing cells resulted in decreased GFP expression. The inventors further confirmed the ability of LacO to repress EF1 promoter by treating cells co-transfected with LacI and bdLV_LacO vectors with Isopropyl β-D-1-thiogalactopyranoside (IPTG) for 24 and 48h. IPTG binds to and represses LacI. Thus, the increase in GFP expression observed when cells are treated with IPTG shows that in the absence of IPTG, LacI binds to Lac operator sequences repressing GFP transcription by the EF1 promoter (FIG. 6Bii).

### Ii) Cloning of the Lac Repressor (LacI) Downstream of PGK Promoter

This step involved the replacement of the puromycin by LacI (in reverse orientation). The LacI was PCR-amplified from the pCMVLac vector (LacSwitch II Inducible Mammalian Expression System from Agilent) and sub-cloned into a smaller vector (pCDN3.1+) to be expressed in the reverse orientation. Next, directed mutagenesis was used to create the restriction enzymes sites necessary to cut out the puromycin cDNA from the bdLV_LacO vector and replace it by LacI.

As shown in FIG. 7, the inventors confirmed that the LacI proteins expressed by the PGK promoter were functional, binding to the Lac operator sequences in bdLV_LacI_LacOand repressing GFP expression by the EF1 promoter. To this end, cells transfected with the bdLV_LacI_LacO vector were treated with IPTG. If the LacI proteins expressed by PGK are functional, treatment with IPTG should increase GFP expression. The inventors observed that treatment with IPTG de-repressed GFP expression in about 50%.

The inventors then cloned four target sites of miR-29a, miR-149, or miR-29a and miR-149 (microRNAs specifically absent or down-regulated in T-ALL cells) downstream of the reporter gene promoter, in the bdLV_LacI_LacO construct, as shown in FIG. 8A. In a second phase, the inventors cloned four target sites of miR-128a or miR-153 (microRNAs specifically up-regulated in T-ALL cells) downstream of the repressor of the reporter gene promoter, in the bdLV_LacI_LacO construct, as shown in FIG. 8B.

### Example 4 - Testing of the MicroRNA-Detector Efficiency and Specificity in Vitro

To evaluate the actual efficiency and specificity of the different microRNA-detectors to selectively target leukemia cells in vitro, the inventors started by performing cultures of T-ALL (‘A’ of FIGS. 9A and 9B) or non-T-ALL (‘B’ of FIGS. 9A and 9B) cells, transduced with the microRNA-detectors BdLV_miR29a_4xT, BdLV_miR149_4xT, and BdLV_miR29a_4xT_miR149_4xT.

Flow cytometry analysis of GFP expression enabled the inventors to determine the efficiency and specificity of each microRNA-detector tested (see FIGS. 9A and 9B). The results show that in cells expressing miR29a or miR149, the microRNA-detector BdLV_miR29a_4xT and BdLV_miR149_4xT (respectively) efficiently repress the expression of the reporter gene (up to 90% of GFP expression repression as compared with BdLV_LacI_LacO transduced cells) (see FIGS. 9A and B). In cells not expressing any of these microRNAs, transduction with the respective microRNA-detector did not affect GFP expression (‘A’ of FIGS. 9A and 9B). Importantly, in cells expressing both miR29a and miR149, transduction with the BdLV_miR29a_4xT_miR149_4xT detector has an additive effect, inducing a repressive effect on GFP expression higher than the detectors BdLV_miR29a_4xT and BdLV_miR149_4xT (‘B’ of FIGS. 9A and 9B).

Referring now to FIGS. 10A and 10B, the inventors then performed cultures of T-ALL cells transduced with the microRNA-detectors BdLV_miR128_4xT,BdLV_miR153_4xT and BdLV_miR128_4xT_miR153_4xT. Flow cytometry analysis of GFP expression showed that in T-ALL cells expressing miR128 and miR153, the microRNA-detector BdLV_miR128_4xT, BdLV_miR153_4xT and BdLV_miR128_4xT_miR153_4xT efficiently de-repressed the expression of the reporter gene (up to a 40 fold increase in GFP expression as compared with BdLV_LacI_LacO transduced cells) (see FIGS. 10A and 10B).

Referring now to FIG. 11, the inventors performed cultures of cells transduced with microRNA-detectors regulated by microRNAs present or up-regulated and absent or down-regulated. As shown, in 293-T non-T-ALL cells that do not express the microRNAs 128 and 153, transduction with the microRNA-detectors BdLV_miR128_4xT_miR29a_4xT, BdLV_miR128_4xT_miR149_4xT, BdLV_miR128_4xT_miR29a_4xT_miR149_4xT and BdLV_miR153_4xT_miR29a_4xT_miR149_4xT resulted in the efficient repression of the reporter gene (up to 90% of GFP expression repression as compared with BdLV_LacI_LacO transduced cells in the microRNA-detectors containing both miR29a and 149 target sites).

As for the CEM T-ALL cells that express miR-128, miR-153 and miR-29a, transduction with the microRNA-detectors BdLV_miR128_4xT_miR29a_4xT, BdLV_miR128_4xT_miR29a_4xT_miR149_4xT and BdLV_miR153_4xT_miR29a_4xT_ miR149_4xT resulted in the efficient repression of the reporter gene. Conversely, transduction with the construct BdLV_miR128_4xT_miR149_4xT, had a small effect on reporter gene expression.

Referring to FIG. 12, in 293-T non-T-ALL cells that do not express the microRNAs 128 and 153, transduction with the microRNA-detectors BdLV_miR-153_4xT-miR-29a_4xT_miR-149_4xT, BdLV_miR-128_4xT-miR-29a_4xT_miR-149_4xT and BdLV miR-128_4xT _miR-153_4xT-miR-29a_4xT_miR-149_4xT resulted in the efficient repression of the reporter gene.

In the DND4.1 T-ALL cells expressing the microRNAs 128 and 153, transduction with the microRNA-detectors BdLV_miR-153_4xT-miR-29a_4xT, BdLV_miR-153_4xT-miR-149_4xT, and BdLV_miR-128_4xT_miR-153_4xT-miR-149_4xT efficiently de-repressed the expression of the reporter gene. As for the CEM T-ALL cells that express miR-128, miR-153 and miR-29a, transduction with the microRNA-detectors BdLV_miR-128_4xT-miR-149_4xT, BdLV_miR-153_4xT-miR-149_4xT and BdLV_miR-128_4xT miR-153_4xT-miR-149_4xT resulted in the efficient de-repression of the reporter gene.

Referring now to FIG. 13, the inventors then performed cultures of T-ALL and non-T-ALL cells transduced with non-integrative lentiviral vectors. As illustrated, in 293-T non-T-ALL cells that do not express the microRNAs 128 and 153, transduction with all of the microRNA-detectors resulted in the efficient repression of the reporter gene. In contrast, in CEM T-ALL cells, transduction with the microRNA-detectors miR-153_4xT-miR-29a_4xT and miR-153_4xT-miR-149_4xT resulted in the efficient de-repression of the reporter gene.

### Discussion & Conclusions

The inventors have generated compelling proof-of-concept data for the feasibility and effectiveness of microRNA-detectors to modulate GFP expression exclusively in T-ALL cells as a practical example of regulation of the activity of an effector gene in the first nucleic acid sequence encoding a therapeutically active molecule or a reporter molecule of the first aspect of the present invention. The inventors anticipate that such a system can be adapted in the future to other cancer types, or even other disease types, with the introduction of the relevant microRNA target sequences to the cancer or disease of interest, thereby paving the way to the use of this gene therapy technology in any disease in which cells display a varied miRNA profile.

The intrinsic ability of this system to distinguish between the identities of a T-ALL cell and that of a non-T-ALL cell within the body of the patient is, hence, a hallmark of the technology and a critically valuable tool in precision oncology. It provides the significant advantage over competing gene therapy technologies developed to date which are applied to precision oncology and it has been deliberately designed to provide the best possible efficacy and safety outcomes for patients. This innovative technology has the potential to effectively circumvent the lack of specificity in T-ALL therapy. Importantly, it serves as a proof-of-concept that this strategy can be applied to the development of similar personalized therapies in other diseases and cancer cell types. In addition, the technology can be effectively use in diagnosis of certain conditions which are characterised by a varied miRNA profile in a disease cell or tissue compared to a healthy cell/tissue.

Finally, with the system of the invention, a therapeutic molecule can be delivered with high specificity and efficiency using just one single vector, and it does not require induction with external factors or cues. Moreover, co-localisation of the encoded molecules in the same cell is achieved at lower dosages than would be possible using a multiple construct approach. As such, this results in a much simpler system that is therefore significantly advantageous over previous technologies.

### REFERENCES

1) Martini, M., Vecchione, L., Siena, S., Tejpar, S., Bardelli, A. (2011) Targeted therapies: how personal should we go. Nature Reviews of Clinical Oncology. Nov 15;9(2):87-97.

2) Pui, C .H., Relling, M.V., Downing, J.R. (2004) Acute lymphoblastic leukemia. New England Journal of Medicine. Apr 8;350(15):1535-48.

3) Fullmer, A., O’Brien, S., Kantarjian, H., Jabbour, E. (2009) Novel therapies for relapsed acute lymphoblastic leukemia. Curr Hematol Malig Rep. Jul;4(3):148-56.

4) Smith, M. A. Update on developmental therapeutics for acute lymphoblastic leukemia (2009)C urr Hematol Malig Rep. 2009 Jul;4(3):175-82.

5) Bartel, D.P. (2004) MicroRNAs: genomics, biogenesis, mechanism, and function. Cell Jan 23;116(2):281-97.

6) Huntzinger, E., Izaurralde, E. (2011) Gene silencing by microRNAs: contributions of translational repression and mRNA decay. Nat Rev Genet Feb;12(2):99-110.

7) Kasinski, A.L., Slack, F.J. (2011) Epigenetics and genetics. MicroRNAs en route to the clinic: progress in validating and targeting microRNAs for cancer therapy. Nat. Rev. C ancer Nov 24;11(12):849-64.

8) Rosenfeld, N., Aharonov, R., Meiri, E., Rosenwald, S., Spector, Y., Zepeniuk, M., Benjamin, H., Shabes, N., Tabak, S., Levy, A., Lebanony, D., Goren, Y., Silberschein, E., Targan, N., Ben-Ari, A., Gilad, S., Sion-Vardy, N., Tobar, A., Feinmesser, M., Kharenko O, Nativ O, Nass D, Perelman M, Yosepovich A, Shalmon B, Polak-C harcon S, Fridman E, Avniel A, Bentwich I, Bentwich Z, C ohen D, C hajut A, Barshack I. (2008) MicroRNAs accurately identify cancer tissue origin. Nat Biotechnol Apr;26(4):462-9.

9) Landgraf, P., Rusu, M., Sheridan, R., Sewer, A., Iovino, N., Aravin, A., Pfeffer, S., Rice, A., Kamphorst, A.O., Landthaler, M., Lin, C., Socci, N.D., Hermida, L., Fulci, V., Chiaretti, S., Foà, R., Schliwka, J., Fuchs, U., Novosel, A., Müller, R.U., Schermer, B., Bissels, U., Inman, J., Phan, Q., Chien, M., Weir, D.B., Choksi, R. et al. (2007) A mammalian microRNA expression atlas based on small RNA library sequencing. Cell Jun 29;129(7):1401-14.

10) Brown, B.D., Venneri, M.A., Zingale, A., Sergi Sergi, L., Naldini, L. (2006) Endogenous microRNA regulation suppresses transgene expression in hematopoietic lineages and enables stable gene transfer. Nat Med May;12(5):585-91.

11) Gentner, B., Visigalli, I., Hiramatsu, H., Lechman, E., Ungari, S., Giustacchini, A., Schira, G., Amendola, M., Quattrini, A., Martino, S., Orlacchio, A., Dick, J.E., Biffi, A., Naldini, L. (2010) Identification of hematopoietic stem cell-specific miRNAs enables gene therapy of globoid cell leukodystrophy. Sci Transl Med. Nov 17;2(58):58ra84

12) Xie, J., Xie, Q., Zhang, H., Ameres, S.L., Hung, J.H., Su, Q., He, R., Mu, X., Seher Ahmed, S., Park, S., Kato, H., Li, C., Mueller, C., Mello, C.C., Weng, Z., Flotte, T.R., Zamore, P.D., Gao, G. (2011) MicroRNA-regulated, systemically delivered rAAV9: a step closer to CNS-restricted transgene expression. Mol Ther. Mar;19(3):526-35.

13) Skalsky, R.L., Cullen, B.R. (2011) Reduced expression of brain-enriched microRNAs in glioblastomas permits targeted regulation of a cell death gene. PLoS One 6(9):e24248.

14) Garzon, R., Fabbri, M., Cimmino, A., Calin, G.A., Croce, C.M. (2006) MicroRNA expression and function in cancer. Trends Mol Med. Dec;12(12):580-7.

15) Lu, J., Getz, G., Miska, E.A., Alvarez-Saavedra, E., Lamb, J., Peck, D., Sweet- Cordero, A., Ebert, B.L., Mak, R.H., Ferrando, A.A., Downing, J.R., Jacks, T., Horvitz, H.R., Golub, T.R. (2005) MicroRNA expression profiles classify human cancers. Nature. Jun 9;435(7043):834-8.

16) Mavrakis, K.J., Van Der Meulen, J., Wolfe, A.L., Liu, X., Mets, E., Taghon, T., Khan, A.A., Setty, M., Rondou, P., Vandenberghe, P., Delabesse, E., Benoit, Y., Socci, N.B., Leslie, C .S., Van Vlierberghe, P., Speleman, F., Wendel, H.G. (2011) A cooperative microRNA tumor suppressor gene network in acute T-cell lymphoblastic leukemia (T-ALL). Nat Genet. 2011 Jun 5;43(7):673-8.

17) Schotte, D., C hau, J.C ., Sylvester, G., Liu, G., C hen, C ., van der Velden, V.H., Broekhuis, M.J., Peters, T.C ., Pieters, R., den Boer, M.L. (2009) Identification of new microRNA genes and aberrant microRNA profiles in childhood acute lymphoblastic leukemia. Leukemia Feb;23(2):313-22.

18) Silva, A., Laranjeira, A.B., Martins, L.R., C ardoso, B.A., Demengeot, J., Yunes, J.A., Seddon, B., Barata, J.T. (2011) IL-7 contributes to the progression of human T-cell acute lymphoblastic leukemias. Cancer Res. Jul 15;71(14):4780-9.

1. A genetic construct comprising:
(i) a first promoter operably linked to a first nucleic acid sequence encoding a therapeutically active molecule or a reporter molecule, wherein the first nucleic acid sequence comprises at least one microRNA (miRNA) target site; and
(ii) a second promoter operably linked to a second nucleic acid sequence encoding an inhibitor of the first promoter and/or the therapeutically active molecule or reporter molecule, and wherein the second nucleic acid sequence comprises at least one miRNA target site, wherein the at least one miRNA target site of the first and second nucleic acid sequences are different.

2. The genetic construct according to claim 1, wherein the at least one miRNA target site of the second nucleic acid sequence is a target site of an miRNA that is different to an miRNA capable of targeting the at least one miRNA target site of the first nucleic acid sequence.

3. The genetic construct according to either claim 1 or claim 2, wherein the first nucleic acid sequence comprises at least one miRNA target site or species of miRNA target site, at least two miRNA target sites or species of miRNA target site, at least three miRNA target sites or species of miRNA target site, at least four miRNA target sites or species of miRNA target site, or at least five miRNA target sites or species of miRNA target site,
optionally wherein there is at least one copy of each miRNA target site, at least two copies of each miRNA target site, at least three copies of each miRNA target site, at least four copies of each miRNA target site, or at least five copies of each miRNA target site, or species of miRNA target site.

4. The genetic construct according to any preceding claim, wherein the at least one miRNA target site present in the first nucleic acid sequence is a target site for a miRNA that is not expressed in a diseased cell.

5. The genetic construct according to claim 4, wherein the diseased cell is a cancer cell, optionally wherein the diseased cell is a T-cell acute lymphoblastic leukaemia (T-ALL) cell.

6. The genetic construct according to any preceding claim, wherein the second nucleic acid sequence comprises at least one miRNA target site or species of miRNA target site, at least two miRNA target sites or species of miRNA target site, at least three miRNA target sites or species of miRNA target site, at least four miRNA target sites or species of miRNA target site, or at least five miRNA target sites or species of miRNA target site,
optionally wherein there is at least one copy of each miRNA target site, at least two copies of each miRNA target site, at least three copies of each miRNA target site, at least four copies of each miRNA target site, or at least five copies of each miRNA target site, or species of miRNA target site.

7. The genetic construct according to any preceding claim, wherein the at least one miRNA target site present in the second nucleic acid sequence is a target site for a miRNA that is expressed in a diseased cell.

8. The genetic construct according to any preceding claim, wherein the reporter molecule is an optical reporter, a nuclear medicine reporter or an MRI reporter.

9. The genetic construct according to any preceding claim, wherein the therapeutically active molecule is a therapeutic protein and/or nucleic acid.

10. The genetic construct according to claim 9, wherein the nucleic acid is DNA, RNA or a chimeric DNA/RNA molecule.

11. The genetic construct according to any one of claims 1 to 9, wherein the therapeutic protein is selected from the group consisting of: an endonuclease, a chimeric antigen receptor, a viral protein and an apoptosis driver protein.

12. The genetic construct according to claim 11, wherein the therapeutic protein is an apoptosis driver protein selected from the group consisting of: Bax, Apoptin, E4orf4 and Bim.

13. The genetic construct according to claim 12, wherein the therapeutic protein is Bax, which comprises or consists of an amino acid sequence as substantially set out in SEQ ID No: 16, or a biologically active variant or fragment thereof.

14. The genetic construct according to claim 12, wherein the therapeutic protein is Apoptin, which comprises or consists of an amino acid sequence as substantially set out in SEQ ID No: 18, or a biologically active variant or fragment thereof.

15. The genetic construct according to claim 12, wherein the therapeutic protein is E4orf4, which comprises or consists of a sequence as substantially set out in SEQ ID No: 20, or a biologically active variant or fragment thereof.

16. The genetic construct according to claim 12, wherein the therapeutic protein is Bim, which comprises or consists of a sequence as substantially set out in SEQ ID No: 25, or a biologically active variant or fragment thereof.

17. The genetic construct according to any preceding claim, wherein the second promoter is arranged in an opposite orientation in the construct to the first promoter.

18. The genetic construct according to any preceding claim, wherein the inhibitor encoded by the second nucleic acid sequence is an inhibitor of the first promoter and wherein the inhibitor of the first promoter is a Lac operon, wherein the second nucleic acid sequence comprises a Lac repressor and the first promoter comprises a Lac operator regulator site.

19. The genetic construct according to any preceding claim, wherein the genetic construct comprises a nucleic acid sequence substantially as set out in any one of SEQ ID Nos: 5, 22 to 24, 27, or 78 to 82, or a fragment or variant thereof.

20. A recombinant vector comprising the genetic construct according to any preceding claim.

21. The genetic construct according to any one of claims 1 to 19, or the vector according to claim 20, for use in therapy.

22. The genetic construct according to any one of claims 1 to 19, or the vector according to claim 20, for use in treating, preventing or ameliorating cancer.

23. A pharmaceutical composition comprising the genetic construct according to any one of claims 1 to 19, or the vector according to claim 20, and optionally a pharmaceutically acceptable vehicle.

24. The genetic construct according to any one of claims 1 to 19, or the vector according to claim 20, for use in diagnosis.

25. The genetic construct according to any one of claims 1 to 19, or the vector according to claim 20, for use in diagnosing cancer.

26. A method of diagnosis or prognosis, the method comprising detecting the reporter molecule of the genetic construct according to any one of claims 1 to 19, or the vector according to claim 20, in a sample obtained from a subject.

27. A method of diagnosing or prognosing cancer in a subject, the method comprising detecting the reporter molecule of the genetic construct according to any one of claims 1 to 19, or the vector according to claim 20, in a sample obtained from a subject.


SEQUENCE LISTING (82 sequences)

SEQ ID NO: 1 - DNA, 22 bp, Homo sapiens
taaccgatttcagatggtgcta

SEQ ID NO: 2 - DNA, 23 bp, Homo sapiens
gggagtgaagacacggagccaga

SEQ ID NO: 3 - DNA, 21 bp, Homo sapiens
agctgcaacatcacaaaatga

SEQ ID NO: 4 - DNA, 21 bp, Homo sapiens
aaagagaccggttcactgtga

SEQ ID NO: 5 - DNA, 10616 bp, Artificial Sequence
  Genetic construct bdLV_LacI_LacO_GFP_miRT4x
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatgtcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgagagtaaaacactacaacgtcgatagcagtaaaacactacaacgtcgacactagtaaaacactacaacgtcgatagcagtaaaacactacaacgtcgacactagtgtcacttggccagagaaatagcagtgtcacttggccagagaaacactagtgtcacttggccagagaaatagcagtgtcacttggccagagaaactcgagtcaaaccttcctcttcttcttaggaggcctcaggctgctctgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatggtgggagggtacctaggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagcgaattcgtaaatataaaatttactaggttgtggaattgtgagcgctcacaattccacagtcgaccctaggttgtggaattgtgagcgctcacaattccacagtcgaccctaggttgtggaattgtgagcgctcacaattccacagtcgaccctagtgtataatgtgttaaactacggatccgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagggatcctaattgtttgtgtattttaggcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctagagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgggtaaccgatttcagatggtgctacgattaaccgatttcagatggtgctatcactaaccgatttcagatggtgctacgattaaccgatttcagatggtgctatcacgggagtgaagacacggagccagacgatgggagtgaagacacggagccagatcacgggagtgaagacacggagccagacgatgggagtgaagacacggagccagacccgggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 6 - RNA, 22 nt, Homo sapiens
uagcaccaucugaaaucgguua

SEQ ID NO: 7 - RNA, 23 nt, Homo sapiens
ucuggcuccgugucuucacuccc

SEQ ID NO: 8 - RNA, 22 nt, Homo sapiens
ucauuuuugugauguugcagcu

SEQ ID NO: 9 - RNA, 21 nt, Homo sapiens
ucacagugaaccggucucuuu

SEQ ID NO: 10 - DNA, 33 bp, Artificial Sequence
  Primer EcoR1_LacO
tgaggcgaattcgtaaatataaaatttactagg

SEQ ID NO: 11 - DNA, 28 bp, Artificial Sequence
  Primer NotI_LacO
tatagcggccgcctaaaatacacaaaca

SEQ ID NO: 12 - DNA, 29 bp, Artificial Sequence
  Primer SmaI_LacI F
tatgcccggggaggtaccctcccaccatg

SEQ ID NO: 13 - DNA, 33 bp, Artificial Sequence
  Primer SmaI_LacI R
ccacccgggtcaaaccttcctcttcttcttagg

SEQ ID NO: 14 - DNA, 25 bp, Artificial Sequence
  Primer HpaI_pcDNA3.1
tgagcgttaacttttaaaagaaaag

SEQ ID NO: 15 - DNA, 32 bp, Artificial Sequence
  Primer SpeI_pcDNA3.1
tataactagtctcgtgcagatggacagcaccg

SEQ ID NO: 16 - protein, 221 aa, Homo sapiens
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWGLPLAESLKRLMSLSPGRPPLLLWDAHVADRDHLCGGSAHRLTHHLEEDGLRPPAALDCVFPP

SEQ ID NO: 17 - DNA, 834 bp, Homo sapiens
gcgctgcggccgcccgcgcggacccggcgagaggcggcggcgggagcggcggtgatggacgggtccggggagcagcccagaggcggggggcccaccagctctgagcagatcatgaagacatgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggggctgcccctggccgagtcactgaagcgactgatgtccctgtctccaggacggcctcctctcctactttgggacgcccacgtggcagaccgtgaccatctttgtggcgggagtgctcaccgcctcactcaccatctggaagaagatgggctgaggcccccagctgccttggactgtgtttttcctccataaattatggcatttttctgggaggggtggggattgggggacgtgggcatttttcttacttttgtaattattggggggtgtggggaagagtggtcttgagggggtaataaacctccttcgggacaca

SEQ ID NO: 18 - protein, 121 aa, Chicken anemia virus
MNALQEDTPPGPSTVFRPPTSSRPLETPHCREIRIGIAGITITLSLCGCANARAPTLRSATADNSESTGFKNVPDLRTDQPKPPSKKRSCDPSEYRVSELKESLITTTPSRPRTARRRIRL

SEQ ID NO: 19 - DNA, 366 bp, Chicken anemia virus
atgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaagaaggcgtataagactgtaa

SEQ ID NO: 20 - protein, 114 aa, Human adenovirus type 2
MVLPALPAPPVCDSQNECVGWLGVAYSAVVDVIRAAAHEGVYIEPEARGRLDALREWIYYNYYTERAKRRDRRRRSVCHARTWFCFRKYDYVRRSIWHDTTTNTISVVSAHSVQ

SEQ ID NO: 21 - DNA, 345 bp, Human adenovirus type 2
atggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtag

SEQ ID NO: 22 - DNA, 11288 bp, Artificial Sequence
  Genetic construct bdLV_LacI_LacO_Bax_GFP_miRT4x
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatgtcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgagagtaaaacactacaacgtcgatagcagtaaaacactacaacgtcgacactagtaaaacactacaacgtcgatagcagtaaaacactacaacgtcgacactagtgtcacttggccagagaaatagcagtgtcacttggccagagaaacactagtgtcacttggccagagaaatagcagtgtcacttggccagagaaactcgagtcaaaccttcctcttcttcttaggaggcctcaggctgctctgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatggtgggagggtacctaggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagcgaattcgtaaatataaaatttactaggttgtggaattgtgagcgctcacaattccacagtcgaccctaggttgtggaattgtgagcgctcacaattccacagtcgaccctaggttgtggaattgtgagcgctcacaattccacagtcgaccctagtgtataatgtgttaaactacggatccgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagggatcctaattgtttgtgtattttaggcggccgccgcgatcgccatggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggggctgcccctggccgagtcactgaagcgactgatgtccctgtctccaggacggcctcctctcctactttgggacgcccacgtggcagaccgtgaccatctttgtggcgggagtgctcaccgcctcactcaccatctggaagaagatgggctgaggcccccagctgccttggactgtgttgcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctagagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgggtaaccgatttcagatggtgctacgattaaccgatttcagatggtgctatcactaaccgatttcagatggtgctacgattaaccgatttcagatggtgctatcacgggagtgaagacacggagccagacgatgggagtgaagacacggagccagatcacgggagtgaagacacggagccagacgatgggagtgaagacacggagccagacccgggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 23 - DNA, 10990 bp, Artificial Sequence
  Genetic construct bdLV_LacI_LacO_Apoptin_GFP_miRT4x
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatgtcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgagagtaaaacactacaacgtcgatagcagtaaaacactacaacgtcgacactagtaaaacactacaacgtcgatagcagtaaaacactacaacgtcgacactagtgtcacttggccagagaaatagcagtgtcacttggccagagaaacactagtgtcacttggccagagaaatagcagtgtcacttggccagagaaactcgagtcaaaccttcctcttcttcttaggaggcctcaggctgctctgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatggtgggagggtacctaggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagcgaattcgtaaatataaaatttactaggttgtggaattgtgagcgctcacaattccacagtcgaccctaggttgtggaattgtgagcgctcacaattccacagtcgaccctaggttgtggaattgtgagcgctcacaattccacagtcgaccctagtgtataatgtgttaaactacggatccgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagggatcctaattgtttgtgtattttaggcggccgcatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaagaaggcgtataagactgtaagcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctagagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgggtaaccgatttcagatggtgctacgattaaccgatttcagatggtgctatcactaaccgatttcagatggtgctacgattaaccgatttcagatggtgctatcacgggagtgaagacacggagccagacgatgggagtgaagacacggagccagatcacgggagtgaagacacggagccagacgatgggagtgaagacacggagccagacccgggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 24 - DNA, 10969 bp, Artificial Sequence
  Genetic construct bdLV_LacI_LacO_E4ORF4_GFP_miRT4x
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatgtcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgagagtaaaacactacaacgtcgatagcagtaaaacactacaacgtcgacactagtaaaacactacaacgtcgatagcagtaaaacactacaacgtcgacactagtgtcacttggccagagaaatagcagtgtcacttggccagagaaacactagtgtcacttggccagagaaatagcagtgtcacttggccagagaaactcgagtcaaaccttcctcttcttcttaggaggcctcaggctgctctgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatggtgggagggtacctaggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagcgaattcgtaaatataaaatttactaggttgtggaattgtgagcgctcacaattccacagtcgaccctaggttgtggaattgtgagcgctcacaattccacagtcgaccctaggttgtggaattgtgagcgctcacaattccacagtcgaccctagtgtataatgtgttaaactacggatccgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagggatcctaattgtttgtgtattttaggcggccgcatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtaggcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctagagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgggtaaccgatttcagatggtgctacgattaaccgatttcagatggtgctatcactaaccgatttcagatggtgctacgattaaccgatttcagatggtgctatcacgggagtgaagacacggagccagacgatgggagtgaagacacggagccagatcacgggagtgaagacacggagccagacgatgggagtgaagacacggagccagacccgggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 25 - protein, 198 aa, Homo sapiens
MAKQPSDVSSECDREGRQLQPAERPPQLRPGAPTSLQTEPQGNPEGNHGGEGDSCPHGSPQGPLAPPASPGPFATRSPLFIFMRRSSLLSRSSSGYFSFDTDRSPAPMSCDKSTQTPSPPCQAFNHYLSAMASMRQAEPADMRPEIWIAQELRRIGDEFNAYYARRVFLNNYQAAEDHPRMVILRLLRYIVRLVWRMH

SEQ ID NO: 26 - DNA, 597 bp, Homo sapiens
atggcaaagcaaccttctgatgtaagttctgagtgtgaccgagaaggtagacaattgcagcctgcggagaggcctccccagctcagacctggggcccctacctccctacagacagagccacaaggtaatcctgaaggcaatcacggaggtgaaggggacagctgcccccacggcagccctcagggcccgctggccccacctgccagccctggcccttttgctaccagatccccgcttttcatctttatgagaagatcctccctgctgtctcgatcctccagtgggtatttctcttttgacacagacaggagcccagcacccatgagttgtgacaaatcaacacaaaccccaagtcctccttgccaggccttcaaccactatctcagtgcaatggcttccatgaggcaggctgaacctgcagatatgcgcccagagatatggatcgcccaagagttgcggcgtattggagacgagtttaacgcttactatgcaaggagggtatttttgaataattaccaagcagccgaagaccacccacgaatggttatcttacgactgttacgttacattgtccgcctggtgtggagaatgcattga

SEQ ID NO: 27 - DNA, 11221 bp, Artificial Sequence
  Genetic construct bdLV_LacI_LacO_BCL2L11_GFP_miRT4x
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatgtcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgagagtaaaacactacaacgtcgatagcagtaaaacactacaacgtcgacactagtaaaacactacaacgtcgatagcagtaaaacactacaacgtcgacactagtgtcacttggccagagaaatagcagtgtcacttggccagagaaacactagtgtcacttggccagagaaatagcagtgtcacttggccagagaaactcgagtcaaaccttcctcttcttcttaggaggcctcaggctgctctgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatggtgggagggtacctaggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagcgaattcgtaaatataaaatttactaggttgtggaattgtgagcgctcacaattccacagtcgaccctaggttgtggaattgtgagcgctcacaattccacagtcgaccctaggttgtggaattgtgagcgctcacaattccacagtcgaccctagtgtataatgtgttaaactacggatccgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagggatcctaattgtttgtgtattttaggcggccgcatggcaaagcaaccttctgatgtaagttctgagtgtgaccgagaaggtagacaattgcagcctgcggagaggcctccccagctcagacctggggcccctacctccctacagacagagccacaaggtaatcctgaaggcaatcacggaggtgaaggggacagctgcccccacggcagccctcagggcccgctggccccacctgccagccctggcccttttgctaccagatccccgcttttcatctttatgagaagatcctccctgctgtctcgatcctccagtgggtatttctcttttgacacagacaggagcccagcacccatgagttgtgacaaatcaacacaaaccccaagtcctccttgccaggccttcaaccactatctcagtgcaatggcttccatgaggcaggctgaacctgcagatatgcgcccagagatatggatcgcccaagagttgcggcgtattggagacgagtttaacgcttactatgcaaggagggtatttttgaataattaccaagcagccgaagaccacccacgaatggttatcttacgactgttacgttacattgtccgcctggtgtggagaatgcattgagcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctagagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgggtaaccgatttcagatggtgctacgattaaccgatttcagatggtgctatcactaaccgatttcagatggtgctacgattaaccgatttcagatggtgctatcacgggagtgaagacacggagccagacgatgggagtgaagacacggagccagatcacgggagtgaagacacggagccagacgatgggagtgaagacacggagccagacccgggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 28 - DNA, 22 bp, Homo sapiens
acacaccaaggataatttctcc

SEQ ID NO: 29 - DNA, 22 bp, Homo sapiens
aactggatgtccctgtatgatt

SEQ ID NO: 30 - DNA, 22 bp, Homo sapiens
aaagaggttaaccatgtattat

SEQ ID NO: 31 - DNA, 22 bp, Homo sapiens
ggttagtggaccgtgttacata

SEQ ID NO: 32 - DNA, 22 bp, Homo sapiens
gaattcaccaagggcaacctct

SEQ ID NO: 33 - DNA, 21 bp, Homo sapiens
aactcctgtatgaagccgttc

SEQ ID NO: 34 - DNA, 22 bp, Homo sapiens
atggcaatatgttggcatagca

SEQ ID NO: 35 - DNA, 22 bp, Homo sapiens
gcacagcaagtgtagacaggca

SEQ ID NO: 36 - DNA, 21 bp, Homo sapiens
aacatacaaagggtatcctct

SEQ ID NO: 37 - DNA, 22 bp, Homo sapiens
ctcccttctttcctcccgtctt

SEQ ID NO: 38 - DNA, 23 bp, Homo sapiens
cacatctgcccccaaaagtccct

SEQ ID NO: 39 - DNA, 22 bp, Homo sapiens
agccaagctcagacggatccga

SEQ ID NO: 40 - DNA, 22 bp, Homo sapiens
tgtgggtgtgtgcatgagcgtg

SEQ ID NO: 41 - DNA, 22 bp, Homo sapiens
tcacaagttagggtctcaggga

SEQ ID NO: 42 - RNA, 22 nt, Homo sapiens
ggagaaauuauccuuggugugu

SEQ ID NO: 43 - RNA, 22 nt, Homo sapiens
aaucauacagggacauccaguu

SEQ ID NO: 44 - RNA, 22 nt, Homo sapiens
auaauacaugguuaaccucuuu

SEQ ID NO: 45 - RNA, 22 nt, Homo sapiens
uauguaacacgguccacuaacc

SEQ ID NO: 46 - RNA, 22 nt, Homo sapiens
agagguugcccuuggugaauuc

SEQ ID NO: 47 - RNA, 21 nt, Homo sapiens
gaacggcuucauacaggaguu

SEQ ID NO: 48 - RNA, 22 nt, Homo sapiens
ugcuaugccaacauauugccau

SEQ ID NO: 49 - RNA, 22 nt, Homo sapiens
ugccugucuacacuugcugugc

SEQ ID NO: 50 - RNA, 21 nt, Homo sapiens
agaggauacccuuuguauguu

SEQ ID NO: 51 - RNA, 22 nt, Homo sapiens
aagacgggaggaaagaagggag

SEQ ID NO: 52 - RNA, 23 nt, Homo sapiens
agggacuuuugggggcagaugug

SEQ ID NO: 53 - RNA, 22 nt, Homo sapiens
ucggauccgucugagcuuggcu

SEQ ID NO: 54 - RNA, 22 nt, Homo sapiens
cacgcucaugcacacacccaca

SEQ ID NO: 55 - RNA, 22 nt, Homo sapiens
ucccugagacccuaacuuguga

SEQ ID NO: 56 - DNA, 24 bp, Homo sapiens
acgtcgcacgaacgcctgtccggg

SEQ ID NO: 57 - DNA, 22 bp, Homo sapiens
gtaatgcaacaaatccccaccc

SEQ ID NO: 58 - DNA, 22 bp, Homo sapiens
ctggaagtgcccatactacagt

SEQ ID NO: 59 - DNA, 18 bp, Homo sapiens
gctcgcccccccgcctca

SEQ ID NO: 60 - DNA, 22 bp, Homo sapiens
gtaagaagtgcttacattgcag

SEQ ID NO: 61 - DNA, 19 bp, Homo sapiens
cacgtcgccgccgaccccg

SEQ ID NO: 62 - DNA, 19 bp, Homo sapiens
aatcagaacgtgacaataa

SEQ ID NO: 63 - DNA, 22 bp, Homo sapiens
aagaccagcaccaattcctcct

SEQ ID NO: 64 - DNA, 24 bp, Homo sapiens
cagccagtattactcatcactttg

SEQ ID NO: 65 - DNA, 21 bp, Homo sapiens
gagccgcgaggagccgagaga

SEQ ID NO: 66 - DNA, 23 bp, Homo sapiens
tctgcaggttctcctgggaccgg

SEQ ID NO: 67 - RNA, 24 nt, Homo sapiens
cccggacaggcguucgugcgacgu

SEQ ID NO: 68 - RNA, 22 nt, Homo sapiens
ggguggggauuuguugcauuac

SEQ ID NO: 69 - RNA, 22 nt, Homo sapiens
acuguaguaugggcacuuccag

SEQ ID NO: 70 - RNA, 18 nt, Homo sapiens
ugaggcgggggggcgagc

SEQ ID NO: 71 - RNA, 22 nt, Homo sapiens
cugcaauguaagcacuucuuac

SEQ ID NO: 72 - RNA, 19 nt, Homo sapiens
cggggucggcggcgacgug

SEQ ID NO: 73 - RNA, 19 nt, Homo sapiens
uuauugucacguucugauu

SEQ ID NO: 74 - RNA, 22 nt, Homo sapiens
aggaggaauuggugcuggucuu

SEQ ID NO: 75 - RNA, 24 nt, Homo sapiens
caaagugaugaguaauacuggcug

SEQ ID NO: 76 - RNA, 21 nt, Homo sapiens
ucucucggcuccucgcggcuc

SEQ ID NO: 77 - RNA, 23 nt, Homo sapiens
ccggucccaggagaaccugcaga

SEQ ID NO: 78 - DNA, 11840 bp, Artificial Sequence
  Genetic construct bdLV_LacI_LacO_GFP_miRT4x_2nd
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatgtcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgaggggcctgtccgcaagcacgctgcatagcgggcctgtccgcaagcacgctgcacactgggcctgtccgcaagcacgctgcatagcgggcctgtccgcaagcacgctgcacactcccacccctaaacaacgtaatgtagccccacccctaaacaacgtaatgcactcccacccctaaacaacgtaatgtagccccacccctaaacaacgtaatgcacttgacatcatacccgtgaaggtctagctgacatcatacccgtgaaggtccacttgacatcatacccgtgaaggtctagctgacatcatacccgtgaaggtccactactccgcccccccgctcgtagcactccgcccccccgctcgcactactccgcccccccgctcgtagcactccgcccccccgctcgcactgacgttacattcgtgaagaatgtagcgacgttacattcgtgaagaatgcactgacgttacattcgtgaagaatgtagcgacgttacattcgtgaagaatgcactgccccagccgccgctgcactagcgccccagccgccgctgcaccactgccccagccgccgctgcactagcgccccagccgccgctgcacctcgagtcaaaccttcctcttcttcttaggaggcctcaggctgctctgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatggtgggagggtacctaggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagcgaattcgtaaatataaaatttactaggttgtggaattgtgagcgctcacaattccacagtcgaccctaggttgtggaattgtgagcgctcacaattccacagtcgaccctaggttgtggaattgtgagcgctcacaattccacagtcgaccctagtgtataatgtgttaaactacggatccgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagggatcctaattgtttgtgtattttaggcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctagagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgggacacaccaaggataatttctcccgatacacaccaaggataatttctcctcacacacaccaaggataatttctcccgatacacaccaaggataatttctcctcacaactggatgtccctgtatgattcgataactggatgtccctgtatgatttcacaactggatgtccctgtatgattcgataactggatgtccctgtatgatttcacaaagaggttaaccatgtattatcgataaagaggttaaccatgtattattcacaaagaggttaaccatgtattatcgataaagaggttaaccatgtattattcacggttagtggaccgtgttacatacgatggttagtggaccgtgttacatatcacggttagtggaccgtgttacatacgatggttagtggaccgtgttacatatcacgaattcaccaagggcaacctctcgatgaattcaccaagggcaacctcttcacgaattcaccaagggcaacctctcgatgaattcaccaagggcaacctcttcacaactcctgtatgaagccgttccgataactcctgtatgaagccgttctcacaactcctgtatgaagccgttccgataactcctgtatgaagccgttctcacatggcaatatgttggcatagcacgatatggcaatatgttggcatagcatcacatggcaatatgttggcatagcacgatatggcaatatgttggcatagcatcacgcacagcaagtgtagacaggcacgatgcacagcaagtgtagacaggcatcacgcacagcaagtgtagacaggcacgatgcacagcaagtgtagacaggcatcacaacatacaaagggtatcctctcgataacatacaaagggtatcctcttcacaacatacaaagggtatcctctcgataacatacaaagggtatcctcttcacctcccttctttcctcccgtcttcgatctcccttctttcctcccgtctttcacctcccttctttcctcccgtcttcgatctcccttctttcctcccgtcttcccgggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 79 - DNA, 12514 bp, Artificial Sequence
  Genetic construct bdLV_LacI_LacO_Bax_GFP_miRT4x_2nd
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatgtcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgaggggcctgtccgcaagcacgctgcatagcgggcctgtccgcaagcacgctgcacactgggcctgtccgcaagcacgctgcatagcgggcctgtccgcaagcacgctgcacactcccacccctaaacaacgtaatgtagccccacccctaaacaacgtaatgcactcccacccctaaacaacgtaatgtagccccacccctaaacaacgtaatgcacttgacatcatacccgtgaaggtctagctgacatcatacccgtgaaggtccacttgacatcatacccgtgaaggtctagctgacatcatacccgtgaaggtccactactccgcccccccgctcgtagcactccgcccccccgctcgcactactccgcccccccgctcgtagcactccgcccccccgctcgcactgacgttacattcgtgaagaatgtagcgacgttacattcgtgaagaatgcactgacgttacattcgtgaagaatgtagcgacgttacattcgtgaagaatgcactgccccagccgccgctgcactagcgccccagccgccgctgcaccactgccccagccgccgctgcactagcgccccagccgccgctgcacctcgagtcaaaccttcctcttcttcttaggaggcctcaggctgctctgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatggtgggagggtacctaggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagcgaattcgtaaatataaaatttactaggttgtggaattgtgagcgctcacaattccacagtcgaccctaggttgtggaattgtgagcgctcacaattccacagtcgaccctaggttgtggaattgtgagcgctcacaattccacagtcgaccctagtgtataatgtgttaaactacggatccgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagggatcctaattgtttgtgtattttaggcggccgctgaatggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggggctgcccctggccgagtcactgaagcgactgatgtccctgtctccaggacggcctcctctcctactttgggacgcccacgtggcagaccgtgaccatctttgtggcgggagtgctcaccgcctcactcaccatctggaagaagatgggctgaggcccccagctgccttggactgtgtttttcctccagcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctagagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgggacacaccaaggataatttctcccgatacacaccaaggataatttctcctcacacacaccaaggataatttctcccgatacacaccaaggataatttctcctcacaactggatgtccctgtatgattcgataactggatgtccctgtatgatttcacaactggatgtccctgtatgattcgataactggatgtccctgtatgatttcacaaagaggttaaccatgtattatcgataaagaggttaaccatgtattattcacaaagaggttaaccatgtattatcgataaagaggttaaccatgtattattcacggttagtggaccgtgttacatacgatggttagtggaccgtgttacatatcacggttagtggaccgtgttacatacgatggttagtggaccgtgttacatatcacgaattcaccaagggcaacctctcgatgaattcaccaagggcaacctcttcacgaattcaccaagggcaacctctcgatgaattcaccaagggcaacctcttcacaactcctgtatgaagccgttccgataactcctgtatgaagccgttctcacaactcctgtatgaagccgttccgataactcctgtatgaagccgttctcacatggcaatatgttggcatagcacgatatggcaatatgttggcatagcatcacatggcaatatgttggcatagcacgatatggcaatatgttggcatagcatcacgcacagcaagtgtagacaggcacgatgcacagcaagtgtagacaggcatcacgcacagcaagtgtagacaggcacgatgcacagcaagtgtagacaggcatcacaacatacaaagggtatcctctcgataacatacaaagggtatcctcttcacaacatacaaagggtatcctctcgataacatacaaagggtatcctcttcacctcccttctttcctcccgtcttcgatctcccttctttcctcccgtctttcacctcccttctttcctcccgtcttcgatctcccttctttcctcccgtcttcccgggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 80 - DNA, 12214 bp, Artificial Sequence
  Genetic construct bdLV_LacI_LacO_Apoptin_GFP_miRT4x_2nd
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatgtcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgaggggcctgtccgcaagcacgctgcatagcgggcctgtccgcaagcacgctgcacactgggcctgtccgcaagcacgctgcatagcgggcctgtccgcaagcacgctgcacactcccacccctaaacaacgtaatgtagccccacccctaaacaacgtaatgcactcccacccctaaacaacgtaatgtagccccacccctaaacaacgtaatgcacttgacatcatacccgtgaaggtctagctgacatcatacccgtgaaggtccacttgacatcatacccgtgaaggtctagctgacatcatacccgtgaaggtccactactccgcccccccgctcgtagcactccgcccccccgctcgcactactccgcccccccgctcgtagcactccgcccccccgctcgcactgacgttacattcgtgaagaatgtagcgacgttacattcgtgaagaatgcactgacgttacattcgtgaagaatgtagcgacgttacattcgtgaagaatgcactgccccagccgccgctgcactagcgccccagccgccgctgcaccactgccccagccgccgctgcactagcgccccagccgccgctgcacctcgagtcaaaccttcctcttcttcttaggaggcctcaggctgctctgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatggtgggagggtacctaggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagcgaattcgtaaatataaaatttactaggttgtggaattgtgagcgctcacaattccacagtcgaccctaggttgtggaattgtgagcgctcacaattccacagtcgaccctaggttgtggaattgtgagcgctcacaattccacagtcgaccctagtgtataatgtgttaaactacggatccgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagggatcctaattgtttgtgtattttaggcggccgcatgaacgctctccaagaagatactccacccggaccatcaacggtgttcaggccaccaacaagttcacggccgttggaaacccctcactgcagagagatccggattggtatcgctggaattacaatcactctatcgctgtgtggctgcgcgaatgctcgcgctcccacgctaagatctgcaactgcggacaattcagaaagcactggtttcaagaatgtgccggacttgaggaccgatcaacccaagcctccctcgaagaagcgatcctgcgacccctccgagtacagggtaagcgagctaaaagaaagcttgattaccactactcccagccgaccccgaaccgcaagaaggcgtataagactgtaagcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctagagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgggacacaccaaggataatttctcccgatacacaccaaggataatttctcctcacacacaccaaggataatttctcccgatacacaccaaggataatttctcctcacaactggatgtccctgtatgattcgataactggatgtccctgtatgatttcacaactggatgtccctgtatgattcgataactggatgtccctgtatgatttcacaaagaggttaaccatgtattatcgataaagaggttaaccatgtattattcacaaagaggttaaccatgtattatcgataaagaggttaaccatgtattattcacggttagtggaccgtgttacatacgatggttagtggaccgtgttacatatcacggttagtggaccgtgttacatacgatggttagtggaccgtgttacatatcacgaattcaccaagggcaacctctcgatgaattcaccaagggcaacctcttcacgaattcaccaagggcaacctctcgatgaattcaccaagggcaacctcttcacaactcctgtatgaagccgttccgataactcctgtatgaagccgttctcacaactcctgtatgaagccgttccgataactcctgtatgaagccgttctcacatggcaatatgttggcatagcacgatatggcaatatgttggcatagcatcacatggcaatatgttggcatagcacgatatggcaatatgttggcatagcatcacgcacagcaagtgtagacaggcacgatgcacagcaagtgtagacaggcatcacgcacagcaagtgtagacaggcacgatgcacagcaagtgtagacaggcatcacaacatacaaagggtatcctctcgataacatacaaagggtatcctcttcacaacatacaaagggtatcctctcgataacatacaaagggtatcctcttcacctcccttctttcctcccgtcttcgatctcccttctttcctcccgtctttcacctcccttctttcctcccgtcttcgatctcccttctttcctcccgtcttcccgggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 81 - DNA, 12193 bp, Artificial Sequence
  Genetic construct bdLV_LacI_LacO_E4ORF4_GFP_miRT4x_2nd
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatgtcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgaggggcctgtccgcaagcacgctgcatagcgggcctgtccgcaagcacgctgcacactgggcctgtccgcaagcacgctgcatagcgggcctgtccgcaagcacgctgcacactcccacccctaaacaacgtaatgtagccccacccctaaacaacgtaatgcactcccacccctaaacaacgtaatgtagccccacccctaaacaacgtaatgcacttgacatcatacccgtgaaggtctagctgacatcatacccgtgaaggtccacttgacatcatacccgtgaaggtctagctgacatcatacccgtgaaggtccactactccgcccccccgctcgtagcactccgcccccccgctcgcactactccgcccccccgctcgtagcactccgcccccccgctcgcactgacgttacattcgtgaagaatgtagcgacgttacattcgtgaagaatgcactgacgttacattcgtgaagaatgtagcgacgttacattcgtgaagaatgcactgccccagccgccgctgcactagcgccccagccgccgctgcaccactgccccagccgccgctgcactagcgccccagccgccgctgcacctcgagtcaaaccttcctcttcttcttaggaggcctcaggctgctctgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatggtgggagggtacctaggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagcgaattcgtaaatataaaatttactaggttgtggaattgtgagcgctcacaattccacagtcgaccctaggttgtggaattgtgagcgctcacaattccacagtcgaccctaggttgtggaattgtgagcgctcacaattccacagtcgaccctagtgtataatgtgttaaactacggatccgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagggatcctaattgtttgtgtattttaggcggccgcatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtaggcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctagagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgggacacaccaaggataatttctcccgatacacaccaaggataatttctcctcacacacaccaaggataatttctcccgatacacaccaaggataatttctcctcacaactggatgtccctgtatgattcgataactggatgtccctgtatgatttcacaactggatgtccctgtatgattcgataactggatgtccctgtatgatttcacaaagaggttaaccatgtattatcgataaagaggttaaccatgtattattcacaaagaggttaaccatgtattatcgataaagaggttaaccatgtattattcacggttagtggaccgtgttacatacgatggttagtggaccgtgttacatatcacggttagtggaccgtgttacatacgatggttagtggaccgtgttacatatcacgaattcaccaagggcaacctctcgatgaattcaccaagggcaacctcttcacgaattcaccaagggcaacctctcgatgaattcaccaagggcaacctcttcacaactcctgtatgaagccgttccgataactcctgtatgaagccgttctcacaactcctgtatgaagccgttccgataactcctgtatgaagccgttctcacatggcaatatgttggcatagcacgatatggcaatatgttggcatagcatcacatggcaatatgttggcatagcacgatatggcaatatgttggcatagcatcacgcacagcaagtgtagacaggcacgatgcacagcaagtgtagacaggcatcacgcacagcaagtgtagacaggcacgatgcacagcaagtgtagacaggcatcacaacatacaaagggtatcctctcgataacatacaaagggtatcctcttcacaacatacaaagggtatcctctcgataacatacaaagggtatcctcttcacctcccttctttcctcccgtcttcgatctcccttctttcctcccgtctttcacctcccttctttcctcccgtcttcgatctcccttctttcctcccgtcttcccgggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 82 - DNA, 12445 bp, Artificial Sequence
  Genetic construct bdLV_LacI_LacO_Bim_GFP_miRT4x_2nd
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatgtcgagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgctcgaggggcctgtccgcaagcacgctgcatagcgggcctgtccgcaagcacgctgcacactgggcctgtccgcaagcacgctgcatagcgggcctgtccgcaagcacgctgcacactcccacccctaaacaacgtaatgtagccccacccctaaacaacgtaatgcactcccacccctaaacaacgtaatgtagccccacccctaaacaacgtaatgcacttgacatcatacccgtgaaggtctagctgacatcatacccgtgaaggtccacttgacatcatacccgtgaaggtctagctgacatcatacccgtgaaggtccactactccgcccccccgctcgtagcactccgcccccccgctcgcactactccgcccccccgctcgtagcactccgcccccccgctcgcactgacgttacattcgtgaagaatgtagcgacgttacattcgtgaagaatgcactgacgttacattcgtgaagaatgtagcgacgttacattcgtgaagaatgcactgccccagccgccgctgcactagcgccccagccgccgctgcaccactgccccagccgccgctgcactagcgccccagccgccgctgcacctcgagtcaaaccttcctcttcttcttaggaggcctcaggctgctctgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcatggtgggagggtacctaggtaagcttgggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaagctagcggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgaccttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatctagagctagcgaattcgtaaatataaaatttactaggttgtggaattgtgagcgctcacaattccacagtcgaccctaggttgtggaattgtgagcgctcacaattccacagtcgaccctaggttgtggaattgtgagcgctcacaattccacagtcgaccctagtgtataatgtgttaaactacggatccgtctcccattaggcctacaatggtgagacaagtagccaacagggaagggttgcaaatatcatttgggcacacctatgataatattgatgaagcagacagtattcagcaagtaactgagaggtgggaagctcaaagccaaagtcctaatgtgcagtcaggtgaatttattgaaaaatttgaggctcctggtggtgcaaatcaaagaactgctcctcagggatcctaattgtttgtgtattttaggcggccgcatggcaaagcaaccttctgatgtaagttctgagtgtgaccgagaaggtagacaattgcagcctgcggagaggcctccccagctcagacctggggcccctacctccctacagacagagccacaaggtaatcctgaaggcaatcacggaggtgaaggggacagctgcccccacggcagccctcagggcccgctggccccacctgccagccctggcccttttgctaccagatccccgcttttcatctttatgagaagatcctccctgctgtctcgatcctccagtgggtatttctcttttgacacagacaggagcccagcacccatgagttgtgacaaatcaacacaaaccccaagtcctccttgccaggccttcaaccactatctcagtgcaatggcttccatgaggcaggctgaacctgcagatatgcgcccagagatatggatcgcccaagagttgcggcgtattggagacgagtttaacgcttactatgcaaggagggtatttttgaataattaccaagcagccgaagaccacccacgaatggttatcttacgactgttacgttacattgtccgcctggtgtggagaatgcattgagcggccgctgagggcagaggaagtcttctaacatgcggtgacgtggaggagaatcccggcccttccggaatggagagcgacgagagcggcctgcccgccatggagatcgagtgccgcatcaccggcaccctgaacggcgtggagttcgagctggtgggcggcggagagggcacccccaagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatcccgcgctcagtcgtccaattctgccgtggacggcaccgccggacccggctccaccggatctcgctagagctgaatctaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcccgggacacaccaaggataatttctcccgatacacaccaaggataatttctcctcacacacaccaaggataatttctcccgatacacaccaaggataatttctcctcacaactggatgtccctgtatgattcgataactggatgtccctgtatgatttcacaactggatgtccctgtatgattcgataactggatgtccctgtatgatttcacaaagaggttaaccatgtattatcgataaagaggttaaccatgtattattcacaaagaggttaaccatgtattatcgataaagaggttaaccatgtattattcacggttagtggaccgtgttacatacgatggttagtggaccgtgttacatatcacggttagtggaccgtgttacatacgatggttagtggaccgtgttacatatcacgaattcaccaagggcaacctctcgatgaattcaccaagggcaacctcttcacgaattcaccaagggcaacctctcgatgaattcaccaagggcaacctcttcacaactcctgtatgaagccgttccgataactcctgtatgaagccgttctcacaactcctgtatgaagccgttccgataactcctgtatgaagccgttctcacatggcaatatgttggcatagcacgatatggcaatatgttggcatagcatcacatggcaatatgttggcatagcacgatatggcaatatgttggcatagcatcacgcacagcaagtgtagacaggcacgatgcacagcaagtgtagacaggcatcacgcacagcaagtgtagacaggcacgatgcacagcaagtgtagacaggcatcacaacatacaaagggtatcctctcgataacatacaaagggtatcctcttcacaacatacaaagggtatcctctcgataacatacaaagggtatcctcttcacctcccttctttcctcccgtcttcgatctcccttctttcctcccgtctttcacctcccttctttcctcccgtcttcgatctcccttctttcctcccgtcttcccgggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg